(19) United States
(12) Patent Application Publication
(10) Pub. No.: US 2023/0183351 A1
(43) Pub. Date: Jun. 15, 2023

(54) A TARGETING MODULE COMPRISING PD-L1 AND/OR PD-L2 FOR USE IN A METHOD FOR STIMULATING A CHIMERIC ANTIGEN RECEPTOR MEDIATED IMMUNE RESPONSE IN A MAMMAL

(71) Applicant: AvenCell Europe GmbH, Dresden (DE)

(72) Inventor: Armin Ehninger, Dresden (DE)

(21) Appl. No.: 17/911,678

(22) PCT Filed: Mar. 16, 2021
(86) PCT No.: PCT/EP2021/056625

§ 371 Date: Sep. 15, 2022

(30) Foreign Application Priority Data
Mar. 16, 2020 (EP) 20163257.7

(51) Int. Cl. C07K 16/28; A61P 37/04; A61K 35/17; C07K 14/705; C07K 14/725

(52) U.S. Cl. CPC C07K 16/2827; A61K 35/17; A61P 37/04; C07K 14/7051; C07K 14/70521; C07K 14/70578; C07K 16/2803; A61K 2039/505; C07K 2317/21; C07K 2317/24; C07K 2317/31; C07K 2317/34; C07K 2319/02; C07K 2319/03; C07K 2319/21; C07K 2319/33; C07K 2319/035; C07K 2319/41

(57) ABSTRACT

The present invention relates to a targeting module comprising at least one PD-L1 and/or PD-L2 binding domain, and a tag-binding domain or a tag for use in a method for stimulating a chimeric antigen receptor mediated immune response in a mammal, a nucleic acid, a vector or a cell comprising a nucleotide sequence encoding the targeting module, a pharmaceutical composition and a kit comprising the targeting module and a vector or a cell comprising a nucleotide sequence encoding a switchable chimeric antigen receptor.

The present invention relates to a targeting module comprising at least one PD-L1 and/or PD-L2 binding domain, and a tag-binding domain or a tag for use in a method for stimulating a chimeric antigen receptor mediated immune response in a mammal, a nucleic acid, a vector or a cell comprising a nucleotide sequence encoding the targeting module, a pharmaceutical composition and a kit comprising the targeting module and a vector or a cell comprising a nucleotide sequence encoding a switchable chimeric antigen receptor.

Chimeric antigen receptors (CARs) are artificial receptors consisting of a binding moiety, which provides the antigen-specificity and one or several signaling chains derived from immune receptors (Cartellieri et al. 2010). These two principal CAR domains are connected by a linking peptide chain including a transmembrane domain, which anchors the CAR in the cellular plasma membrane. Immune cells, in particular T and NK cells, can be genetically modified to express CARs inserted into their plasma membrane. If such a CAR-modified immune cell encounters other cells or tissue structures expressing or being decorated with the appropriate target of the CAR binding moiety, upon binding of the CAR binding moiety to the target antigen the CAR modified immune cell is cross-linked to the target. Cross-linking leads to an induction of signal pathways via the CAR signaling chains, which will change the biologic properties of the CAR-engrafted immune cell. For example, CAR triggering in effector CD4+ and CD8+ T cells will activate typical effector functions like secretion of lytic compounds and cytokines, which will eventually lead to the killing of the respective target cell. In contrast, CAR activation in gene-modified regulatory T cells (Tregs) leads to an activation of Treg-specific immunomodulatory and suppressive mechanism like interleukin (IL)-10 or tumor growth factor beta (TGF-β) secretion. The adoptive transfer of immune cells engineered with chimeric antigen receptors (CARs) is currently considered as a highly promising therapeutic option for treatment of otherwise incurable malignant, infectious or autoimmune diseases. Until today, two CAR-T cell therapeutics have gained market approval for treatment of B cell derived malignancies, proving the clinical feasibility of this approach.

However, the conventional CAR technology comes along with a number of critical issues, which need to be solved before this treatment modality can be widely applied for clinical treatments. First of all, several safety issues have to be addressed. So far, immune responses of T cells engineered with conventional CARs are difficult to control after infusion into the patient. Serious adverse event rates are high (Titov et al. 2018). Especially unexpected target gene expression on normal tissue may provoke a rapid and rigorous immune reaction of engineered T cells against normal cells, which can cause severe side effects (Lamers et al. 2006, Morgan et al. 2010). Moreover, as CAR-T cells are a new class of self-amplifying cell drugs, infused T cells can undergo a vigorous expansion in the presence of heavy tumor burden leading to tumor lysis syndrome, cytokine release syndrome and macrophage activation syndrome (Brudno and Kochenderfer 2016, Maude et al. 2014). Another drawback of conventional CAR technology is the restriction of engineered T cell retargeting to a single antigen. Such a monotherapeutic approach implies the risk for development of tumor escape variants, which have lost the target antigen during treatment. The emergence of tumor escape variants under conventional CAR T cell therapy after several months was already observed in clinical trials (Grupp et al. 2013, Sotillo et al. 2015). Taken together, these obstacles restrict the application of CAR-T cells to very few indications. In fact, examples of clinical effectiveness have been restricted to CD19- and BCMA-targeting CAR-T cells until now.

Modular “universal” CAR-T (UniCAR) approaches can overcome these limitations by separating antigen-recognition and activating domain of a CAR into two separate operational units. T cells are engineered to express a CAR with a universal binding domain recognizing a tag (Cartellieri et al. 2016). Antigen-specificity is provided by soluble adapter proteins, which consist of an antigen-binding domain fused to the tag recognized by the universal CAR. Cartellieri et al. describe the treatment of CD33- and/or CD123-positive acute myeloid leukaemia cells in vitro and in vivo.

WO 2012/082841 A2 discloses universal anti-tag chimeric antigen receptor-expressing T cells and methods of treating cell-related disorders, e.g. cancer. In addition, WO 2013/044225 A1 discloses a universal immune receptor expressed by T cells for the targeting of diverse and multiple antigens. Both methods describe the use of modified T cells expressing universal anti-tag immune receptors. These T cells can be redirected to disease-related cell surface antigens by additionally applying soluble modules binding these surface antigens and carrying the respective tag. WO 2016/030414 A1 provides a genetically modified immune cell that allows a redirection against diverse disorders in a safe and efficient manner using endogenous tags based on nuclear proteins.

WO 2018/224660 A1 describes target modules for universal anti-tag chimeric antigen receptor-expressing T cells comprising chemically synthesised peptide binding units specific for a human surface protein or protein complex and their advantages, in particular the fast and easy preparation, the improved stability and good pharmaceutical properties. WO 2016/168773 A2 discloses a peptide neo-epitope (PNE)-based switch for CAR-T cells comprising a receptor-binding partner, in particular a peptide antigen, a target-binding moiety, in particular a soluble T cell receptor or a part thereof or an antibody fragment; and a linker.

WO 2015/057834 A1 discloses switchable CAR-T cells and a switch for CAR-T cells comprising a receptor-binding partner, in particular a peptide antigen. The peptide antigen may be a natural, non-natural or artificial peptide, in particular yeast transcription factor GCN4 or a homologue thereof. Furthermore, the switch comprises a target-binding moiety, in particular an antibody or anti-body fragment, preferably selected from an anti-Her2 antibody, an anti-BCMA antibody, an anti-CD19 antibody, an anti-CEA antibody and fragments thereof, and a linker.

Yu et al. describes different generations of CAR-T cells and strategies to reduce toxicity, including the so-called cytokine release syndrome and on-target off-tumour toxicity (Yu et al. 2019), in particular switchable CAR-T cells, in particular by suicide genes, for example herpes simplex virus thymidine kinase (HSV-tk) or inducible caspase 9 (iCasp9) or an universal CAR system using FITC-binding CAR-T cells and small molecules comprising FITC and an antibody for a tumor target, in particular cetuximab, trastuzumab or rituximab.

Further, Yu et al. discloses PD-1 inhibiting CAR-T cells (iCAR) and points out the disadvantages of i-CAR-T cells, in particular that iCAR-T cells recognize only tissue-specific antigens that are absent or down-regulated on tumors and expressed by the off-target tissue, and that no temporal and spatial control of iCAR-T cells is possible.

WO 2018/078066 A1 discloses an anti-linker/label epitope (anti-LLE) extracellular CAR comprising a linker/label epitope binding domain, a transmembrane domain and a signal transduction domain, wherein the extracellular linker/label epitope binding domain binds a target cell binding molecule (TCBM) comprising an antigen binding moiety (ABM), a naturally occurring label epitope (LaM), and a linker (LiM), wherein the ABM specifically binds an antigen on a target cell, preferably a tumor antigen, and wherein the linker connects the ABM and the LaM and forms a linker/label epitope, wherein the extracellular LLE binding domain binds the LLE with a higher preference than the naturally occurring LaM.

Despite remarkable therapeutic success using chimeric antigen receptor modified T cells (CAR-T) in hematologic malignancies, targeting solid tumors still remains challenging, given their heterogeneity and immunosuppressive milieu, including the expression of inhibitory immune checkpoints such as PD-L1 or PD-L2 by tumor cells. Preclinical models demonstrate that combining CAR-T therapy with checkpoint inhibitors targeting the PD-⅟PD-L1 axis could be a promising strategy to enhance anti-cancer activity of CAR-T in solid tumors and on-going phase I trials are clinically evaluating this strategy (Grosser et al. 2019). However, primary and acquired resistance to checkpoint blockade is frequently observed limiting its broad applicability and efficacy (tumor escape variants). In addition, lack of specificity results in frequent occurrence of immune-related adverse events affecting several organs.

Rafiq et al. disclose a method for targeted delivery of a PD-1-blocking scFv by CAR-T cells to enhance anti-tumour efficacy in vivo (Rafiq et al. 2018). Furthermore, Rafiq et al. describes a comparison with a combination therapy comprising CAR-T cells and an immune checkpoint blockade inhibitor, in particular a PD-1 blocking mAb; and an increase of the efficacy of CAR-T cell therapy, in particular increasing the survival of PD-L1 tumor-bearing mice by treatment with CAR constructs against CD19 or MUC16^{ecto} comprising a c-myc tagged scFv obtained from the variable heavy and light chain of a PD-1 blocking mAb.

Further, Rafiq et al. disclose as alternative methods for preventing PD-1-induced attenuation of CAR-T cell responses co-expression of a chimeric switch receptor in which the extracellular domain of PD-1 is linked to activation signaling domains, or co-modification of CAR-T cells to express a dominant-negative PD-1 receptor, and the disadvantages of these methods, in particular that the protection is limited to the T cells themselves. The latter approach of expressing a dominant-negative PD-1 receptor is also disclosed by Cherkassky et al. (Cherkassky et al. 2017).

Prosser et al. describes modified primary human CD8+ cytotoxic T cells expressing a PD-1 and CD28 chimeric receptor, produced by conversion of PD-1 into a co-stimulatory receptor using the transmembrane and cytoplasmic domains of CD28, while retaining the ability of the receptor to bind PD-L1, for reversing the suppressive tumour PD-L1 effect on T cells (Prosser et al. 2012). Furthermore, Prosser et al. discloses the co-stimulation of the modified T cells by PD-L1 tumors. Disadvantageously, the modified T cells with a chimeric PD-1:CD28 receptor always receive a costimulatory signal when they are exposed to PD-L1 or PD-L2 positive cells. As PD-L1 is not only expressed by tumor cells, but also by antigen-presenting cells, this chronic stimulation increases the risk of the development of tumor escape variants and non-specific effects (i.e. autoimmunity) as well as the oncogenic transformation risk.

The object of the present invention is to provide an approach, which overcomes the disadvantages of the state of the art.

The object is solved by a targeting module comprising
i) at least one PD-L1 and/or PD-L2 binding domain,
ii) a tag-binding domain or a tag,
for use in a method for stimulating a chimeric antigen receptor mediated immune response in a mammal, preferably for use in the treatment of cancer, infectious disease or autoimmune disease; wherein the targeting module is administered in combination with a vector or a cell comprising a nucleotide sequence encoding a switchable chimeric antigen receptor, wherein the switchable chimeric antigen receptor comprises three domains, wherein
the first domain is a tag-binding domain or a tag,
the second domain is an extracellular hinge and a transmembrane domain and
the third domain is a signal transduction domain,
wherein the tag-binding domain of the targeting module binds to the tag of the switchable chimeric antigen receptor or the tag of the targeting module binds to the tag-binding domain of the switchable chimeric antigen receptor.

Advantageously, the targeting module according to the invention used in combination with a switchable chimeric antigen receptor actively targets PD-L1 and/or PD-L2 expressed by tumor cells and is capable of inducing a significant anti-tumor response, wherein the anti-tumor response of the switchable chimeric antigen receptor is only been induced in the presence of the targeting module. The effect can be interrupted immediately by withholding interrupting the administration of the targeting module. Further advantageously, the pharmacokinetic and pharmacodynamic half-life of the targeting module according to the invention is short, preferably in the range of 10 min to 5 h, providing a rapid and reversible switch off mechanism of the mediated immune response. Advantageously, the toxicity of the targeting module according to the invention is low, which is a result of the rapid switch off mechanism, as can be demonstrated by sparing PD-L1 low expressing cells when targeting module is not replenished in an in vitro experiment.

As used herein, the term “targeting module” refers to an isolated protein with at least two different domains, wherein each domain is specific for a target or a uniform group of targets, respectively, wherein at least one domain is specific for a target cell, in particular the PD-L1 and/or PD-L2 binding domain; and one domain is specific for a switchable chimeric antigen receptor, in particular the tag-binding domain or the tag.

According to the invention, the targeting module comprises at least one PD-L1 binding domain or at least one PD-L2 binding domain or at least one PD-L1 binding domain and at least one PD-L2 binding domain.

As used herein “PD-L1” (Programmed death ligand 1) refers to a transmembrane protein according to SEQ ID No. 1 that in humans is encoded by the CD274 or PDCD1LG1 gene and is a ligand of PD-1. As used herein “PD-L2” (Programmed death 1 ligand 2) refers to a transmembrane protein according to SEQ ID No. 2 that in humans is encoded by the CD273 or PDCD1LG2 gene and is a ligand of PD-1.

As used herein, the term “domain” refers to a part of a protein sequence, which can exist and function independently from the rest of the protein.

As used herein, the term “specific” refers to the ability of an antibody or antibody fragment or a protein, peptide or low molecular weight organic ligand to recognize and bind with a binding partner (e.g. a tumor antigen) protein present in a sample, but not substantially recognize or bind other molecules in the sample.

As used herein, the term “binds” or “binding” refers to a non-covalent binding, in particular ionic bonds, hydrogen bonds, Van der Waals forces and/or hydrophobic interactions.

As used herein, the term “administered in combination” refers to a treatment, wherein the targeting module is administered prior to, simultaneously with and/or after the administration of the vector or cell comprising a nucleotide sequence encoding a switchable chimeric antigen receptor.

In embodiments, the targeting module is administered on its own, preferably one hour to 2 days, more preferably 4 to 24 hours, prior to the administration of the vector or cell comprising a nucleotide sequence encoding a switchable chimeric antigen receptor. Advantageously, the administration of the targeting module prior to the administration of the vector or cell comprising a nucleotide sequence encoding a switchable chimeric antigen receptor stimulates the switchable chimeric antigen receptors and increases the expansion of the switchable chimeric antigen receptor carrying effector cells and their accumulation at the target site.

In further embodiments, the targeting module is administered simultaneously with the vector or cell comprising a nucleotide sequence encoding a switchable chimeric antigen receptor.

In further embodiments, the targeting module is administered, preferably in the range of 3 days to 30 days, after the administration of the vector or cell comprising a nucleotide sequence encoding a switchable chimeric antigen receptor. Furthermore, additional such doses of the targeting module may be administered following resting periods to reactivate the switchable chimeric antigen receptor carrying effector cells.

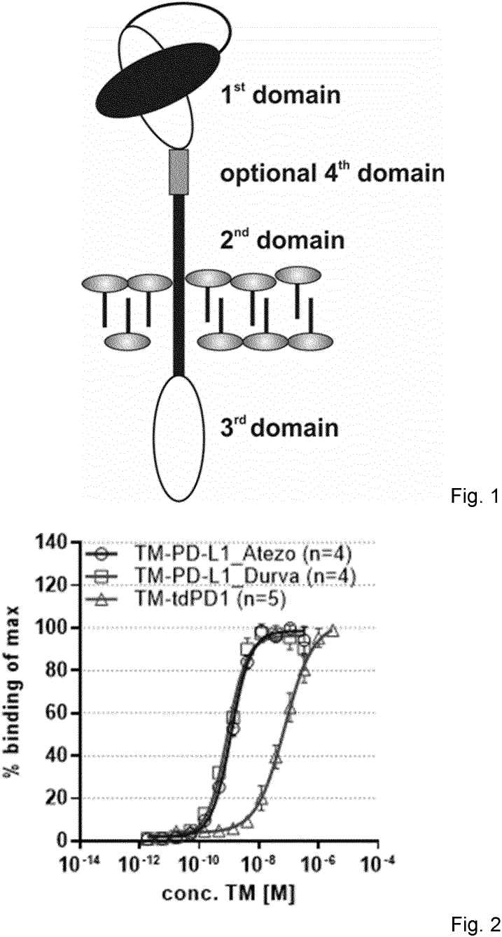

As used herein, the term “switchable chimeric antigen receptor” refers to an artificial chimeric fusion protein, in particular a receptor comprising a tag-binding domain or a tag, an extracellular hinge and a transmembrane domain and a signal transduction domain (FIG. 1). The domains can be derived from different sources and therefore, the receptor is called chimeric. Advantageously, the receptor can bind with the tag-binding domain or tag to different targeting modules.

Advantageously, the cell comprising a nucleotide sequence encoding a switchable chimeric antigen receptor (CAR) expresses the switchable CAR, which has binding specificity for the tag-binding domain or tag of the targeting module, which in turn binds to PD-L1 and/or PD-L2 on a target cell.

The term “autoimmune disorder” refers to an abnormal immune response of the body against substances and tissues normally present in the body (autoimmunity).

In embodiments, the targeting module is in monomeric or dimeric form, preferably in monomeric form.

In further embodiments, the targeting module is monovalent or bivalent.

In some embodiments, the targeting module according to the invention is bivalent and comprises one PD-L1 and one PD-L2 binding domain.

Preferably, the PD-L1 binding domain is an antibody or antibody fragment.

Preferably, the PD-L2 binding domain is an antibody or antibody fragment.

As used herein, the term “antibody” refers to a protein, which binds antigens via the Fab’s variable region. The fragment antigen-binding (Fab) fragment is a region on an antibody that binds to antigens. It is composed of one constant and one variable domain of each of the heavy (V_{H}) and the light chain (V_{L}). As used herein, the term “antibody fragment” refers to a protein comprising at least the variable domain of a light or heavy chain of an antibody. In an embodiment, antibody fragments are selected from single-chain variable fragments (scFv), single chain antibodies, F(ab′)2 fragments, Fab fragments, and fragments produced by a Fab expression library or single-domain antibodies (nanobodies).

As used herein, the term “single chain variable fragment (scFv)” refers to an artificial antibody fragment comprising a variable domain of a light and a heavy chain of an antibody covalently linked. In an embodiment, the variable domain of a light (V_{L}) and a heavy chain (V_{H}) of an antibody are covalently linked by a short peptide of ten to 25 amino acids. In a further embodiment, the short peptide links the N-terminus of the V_{H} with the C-terminus of the V_{L}, or vice versa.

In embodiments, the antibody is obtained from an animal species, preferably from a mammal such as human, simian, mouse, rat, rabbit, guinea pig, horse, cow, sheep, goat, pig, dog or cat. Preferably, the antibody or antibody fragment is a human, humanized or deimmunized antibody. Humanized antibodies can be prepared in various ways, for example, by resurfacing and CDR grafting. In case of resurfacing, a combination of molecular modeling, statistical analyses, and mutagenesis is used to modify all non-CDR regions on the surface of the antibody to become similar to the surface of antibodies of the target organism. In CDR grafting, the CDR regions according to the invention are introduced into known human framework regions, which are similar in sequence to the original ones. Deimmunized antibodies can be obtained by specifically mutating residues that confer immunogenicity hotspots as predicted based on in silico peptide-MHC affinity prediction.

In embodiments, the antibody or antibody fragment is a polyclonal, a monoclonal, or a chimeric antibody, wherein an antigen binding region of a non-human antibody is transferred into the framework of a human antibody by recombinant DNA techniques including in silico design.

In embodiments, antibodies to a selected tag or antigen may be produced by immunization of various hosts including, but not limited to, goats, rabbits, rats, mice, humans, through injection with cells expressing a particular protein, DNA or RNA encoding for the protein, the protein itself or any portion, fragment or oligopeptide that retain immunogenic properties of the protein.

In embodiments, the PD-L1 binding domain is selected from PD-1, BMS-936559, Atezolizumab, Durvalumab, Avelumab, 3F2.1 and fragments thereof. Preferably, the PD-L1 binding domain is an scFv fragment of BMS-936559, Atezolizumab or Durvalumab.

As used herein, the term “fragments” refers to proteins consisting of at least 90%, preferably at least 95%, more preferably at least 98% of the amino acid sequence of the PD-L1 binding domains.

As used herein, the term “PD-1” (Programmed cell death protein 1) refers to a protein on the surface of cells that has a role in regulating the immune system’s response to the cells of the human body by down-regulating the immune system and promoting self-tolerance by suppressing T cell inflammatory activity. Preferably, the PD-L1 binding domain comprises at least one sequence with a sequence identity of at least 90%, preferably at least 95%, more preferably at least 98% to SEQ ID No. 3 or a fragment thereof, wherein the at least one sequence binds the antigen PD-L1 and PD-L2. In embodiments, the fragment of SEQ ID No. 3 comprises the extracellular domain of PD-1.

As used herein, the term “BMS-936559” refers to an antibody that specifically binds PD-L1. Preferably, the PD-L1 binding domain comprises at least one sequence each with a sequence identity of at least 90%, preferably at least 95%, more preferably at least 98% to SEQ ID No.4 and SEQ ID No. 5 or SEQ ID No. 67 and SEQ ID No. 68 or SEQ ID No. 69 and SEQ ID No. 70 or SEQ ID No. 71 and SEQ ID No. 72 or SEQ ID No. 73 and SEQ ID No. 74 or SEQ ID No. 75 and SEQ ID No. 76 or SEQ ID No. 77 and SEQ ID No. 78 or SEQ ID No. 79 and SEQ ID No. 80 or SEQ ID No. 81 and SEQ ID No. 82 or SEQ ID No. 83 and SEQ ID No. 84, wherein the at least one sequence binds the antigen PD-L1.

More preferably, the PD-L1 binding domain comprises at least one sequence each with a sequence identity of at least 90%, preferably at least 95%, more preferably at least 98% toSEQ ID No. 4 and SEQ ID No. 5 or SEQ ID No. 77 and SEQ ID No. 78, wherein the at least one sequence binds the antigen PD-L1.

In embodiments, the PD-L1 binding domain comprises at least one sequence each with a sequence identity of at least 90%, preferably at least 95%, more preferably at least 98% to one sequence of the group comprising SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96, SEQ ID No. 97, SEQ ID No. 98, SEQ ID No. 99, SEQ ID No. 100, SEQ ID No. 101, SEQ ID No. 102, SEQ ID No. 103 and SEQ ID No. 104.

Preferably, the PD-L1 binding domain comprises at least one sequence each with a sequence identity of at least 90%, preferably at least 95%, more preferably at least 98% to SEQ ID No. 86 or SEQ ID No. 97.

As used herein, the term “Atezolizumab” refers to an antibody that specifically binds PD-L1. Preferably, the PD-L1 binding domain comprises at least one sequence each with a sequence identity of at least 90%, preferably at least 95%, more preferably at least 98% to SEQ ID No. 6 and SEQ ID No. 7, wherein the at least one sequence binds the antigen PD-L1.

In embodiments, the PD-L1 binding domain comprises at least one sequence each with a sequence identity of at least 90%, preferably at least 95%, more preferably at least 98% toSEQ ID No. 57 or SEQ ID No. 65.

As used herein, the term “Durvalumab” refers to an antibody that specifically binds PD-L1. Preferably, the PD-L1 binding domain comprises at least one sequence each with a sequence identity of at least 90%, preferably at least 95%, more preferably at least 98% toSEQ ID No. 8 and SEQ ID No. 9, wherein the at least one sequence binds the antigen PD-L1.

In embodiments, the PD-L1 binding domain comprises at least one sequence each with a sequence identity of at least 90%, preferably at least 95%, more preferably at least 98% toSEQ ID No. 58 or SEQ ID No. 66.

As used herein, the term “Avelumab” refers to an antibody that specifically binds PD-L1. Preferably, the PD-L1 binding domain comprises at least one sequence each with a sequence identity of at least 90%, preferably at least 95%, more preferably at least 98% to SEQ ID No. 61 and SEQ ID No. 62 or SEQ ID No. 105 and SEQ ID No. 106, wherein the at least one sequence binds the antigen PD-L1.

In embodiments, the PD-L1 binding domain comprises at least one sequence each with a sequence identity of at least 90%, preferably at least 95%, more preferably at least 98% to SEQ ID No. 107 or SEQ ID No. 108.

As used herein, the term “3F2.1” refers to an antibody that specifically binds PD-L1. Preferably, the PD-L1 binding domain comprises at least one sequence each with a sequence identity of at least 90%, preferably at least 95%, more preferably at least 98% to SEQ ID No. 63 and SEQ ID No. 64, wherein the at least one sequence binds the antigen PD-L1.

As used herein, the term “tag” refers to a peptide sequence attached to peptides or proteins to enable them to bind to specific atoms, ions or molecules, in particular the tag-binding domain.

In embodiments, the PD-L2 binding domain is selected from an antibody or fragment thereof, which specifically binds PD-L2.

In embodiments, the tag is a peptide epitope tag. In further embodiments, the tag comprises 10 to 20 amino acids.

In embodiments, the peptide epitope tag is a myc-tag, a His-tag, a short linear peptide sequence from yeast transcription factor GCN4, preferably according to SEQ ID No. 10, SEQ ID No. 11 or mutants thereof; a leucine zipper sequence, preferably SYNZIP 1 to SYNZIP 48, BATF, FOS, ATF4, ATF3, BACH1, JUND, NFE2L3, HEPTAD (Reinke et al. 2010), a sequence according to SEQ ID No. 12 or SEQ ID No. 13 or mutants thereof; or a short linear peptide sequence from a human nuclear protein, preferably from the human La protein. Preferably, the peptide epitope tag from the human La protein is the human La epitope E5B9 according to SEQ ID No. 14 or E7B6 according to SEQ ID No. 15 or SEQ ID No. 16, most preferably the human La epitope E5B9 according to SEQ ID No. 14 or E7B6 according to SEQ ID No. 16.

As used herein, the term “mutants” refers to peptides or proteins having at least 85% sequence identity to the named peptides or proteins, preferably at least 95% sequence identity. Advantageously, the mutants are capable of having one or more activities of the named peptides or proteins, in particular bind the identical tag-binding domains as the peptide epitope tag.

In embodiments, mutants are truncated versions of peptides or proteins. As used herein, the term “truncated versions” refers to shortened peptides or proteins having at least 85% sequence identity to the named peptides or proteins, preferably at least 95% sequence identity, more preferably having a chain length of at least 85% and a sequence identity of 100%, most preferably a chain length of at least 95% and a sequence identity of 100%. Advantageously, the truncated version has at least 80%, preferably of at least 90%, more preferably of at least 95%; of the activity of the named peptide or protein.

As used herein, the term “nuclear protein” refers to a protein found in the cell nucleus.

In embodiments, the His-tag is an amino acid sequence consisting of histidine residues, preferably in the range of six to fourteen histidine residues.

Advantageously, tags, which are peptide sequences from nuclear antigens, cannot be accessed and bound by the corresponding tag-binding domain in the context of the native protein under physiological conditions. Further advantageously, the tag is not immunogenic. This leads to minimization of risk of uncontrolled on-target off-site toxicities by CAR expressing immune cells like release of toxic levels of cytokines, referred to variously as cytokine storms or cytokine release syndrome (CRS).

In preferred embodiments, the peptide epitope tag is a myc-tag, a His-tag, a short linear peptide sequence from yeast transcription factor GCN4, preferably according to SEQ ID No. 10, SEQ ID No. 11; a leucine zipper sequence, preferably SYNZIP 1 to SYNZIP 48, BATF, FOS, ATF4, ATF3, BACH1, JUND, NFE2L3, HEPTAD (Reinke et al. 2010), a sequence according toSEQ ID No. 12 or SEQ ID No. 13; or a short linear peptide sequence from a human nuclear protein, preferably from the human La protein. Preferably, the peptide epitope tag from the human La protein is the human La epitope E5B9 according to SEQ ID No. 14 or E7B6 according to SEQ ID No. 15 or SEQ ID No. 16, most preferably the human La epitope E5B9 according to SEQ ID No. 14 or E7B6 according to SEQ ID No. 16.

In further embodiments, the tag-binding domain is an antibody or an antibody fragment.

In embodiments, the tag-binding domain binds to a tag from the human nuclear La protein, preferably the tag-binding domain is an antibody or an antigen-binding fragment, wherein the tag-binding domain constitutes an anti-La epitope scFv, more preferably an anti-La epitope scFv comprising a V_{L} according to the following sequence:

DIVMTQSPDSLAVSLGERATINCX¹SSQSLLNSRTX²KNYLAWYQQ
KPGQPPKLLIYWASTRX³SGVPDRFSGSGSGTDFTLTISSLQAEDVAV
YYCKQSYNLX⁴TFGGGTKVEIK(SEQ ID No. 17),

wherein X¹ to X⁴ are variable amino acid residues, preferably selected from a proteinogenic alpha amino acid residue; or a sequence having at least 85% sequence identity, preferably at least 95% sequence identity; to one of the sequences SEQ ID No. 19 or SEQ ID No. 21.

In some embodiments, the variable amino acid residues X¹ to X⁴ are selected as follows:
X¹ is selected from polar and/or positive charged residues, such as Serine, Threonine, Asparagine, Glutamine, Histidine, Lysine and Arginine; preferably Lysine or Arginine;
X² is preferably selected from Lysine and Proline;
X³ is selected from polar and charged residues, such as Asparagine, Aspartic Acid, Glutamine, Glutamic acid, Histidine, Lysine and Arginine, preferably Glutamic acid and Lysine;
X⁴ is selected from hydrophobic residues, such as Isoleucine, Leucine, Valine, Alanine, Methionine, Phenylalanine, Proline and Tryptophan; preferably Leucine or Proline.

In further embodiments, the tag-binding domain is an antibody or an antigen-binding fragment, wherein the tag-binding domain constitutes an anti-La epitope scFv comprising a sequence having at least 85% sequence identity, preferably at least 95% sequence identity; to one of the sequences SEQ ID No. 18 or SEQ ID No. 20.

Preferably, the tag-binding domain constitutes an anti-La epitope scFv comprising a sequence having each at least 85% sequence identity, preferably at least 95% sequence identity; to the sequences according to SEQ ID NO. 18 (V_{H}) and SEQ ID NO. 19 (V_{L}) or the sequences according to SEQ ID NO. 20 (V_{H}) and SEQ ID NO. 21 (V_{L}).

Most preferably, the tag-binding domain constitutes the anti-La 5B9 scFv according to SEQ ID NO. 18 (V_{H}) and SEQ ID NO. 19 (V_{L}) or the anti-La 7B6 scFv according to SEQ ID NO. 20 (V_{H}) and SEQ ID NO. 21 (V_{L}).

In further embodiments, the targeting module according to the invention further comprises an effector cell binding domain, wherein the effector cell binding domain specifically binds an epitope on a human CD3 chain, a human T cell receptor (TCR) or human CD16.

As used herein, the term “effector cell binding domain” refers to a peptide or protein, which specifically binds a protein or protein complex (antigen) on an effector cell. As used herein, the term “protein complex” refers to a group of two or more associated protein chains.

As used herein, the term “effector cell” refers to an immune cell, which executes immune reactions, in particular an immune cell with cytolytic, phagocytic and/or immunosuppressive activity. Preferably, the effector cell is a T cell, a natural killer (NK) cell or a macrophage.

Advantageously, the targeting module activates the effector cell by binding to the domain and exceeding a certain threshold regarding required number of such interactions.

Preferably, the effector cell binding domain is an antibody or antibody fragment.

In an embodiment, the effector cell binding domain specifically binds an epitope on human CD3 with a dissociation constant K_{d} up to 10⁻⁸ M. Commonly, the affinity of the effector cell binding domain, in particular the dissociation constant K_{d}, is determined with surface plasmon resonance measurements or flow-cytometry-based cellular binding assay.

Preferably, the effector cell binding domain specifically binds an epitope on human CD3 with a low affinity, preferably with a dissociation constant K_{d} in the range of 10⁻⁴ M to 10⁻⁸ M.

In embodiments, the effector cell binding domain comprises at least one of the following sequences:
i) a V_{L} according to SEQ ID No. 22 or 24 and/or
ii) a V_{H} according to SEQ ID No. 23 or 25.

In embodiments, the effector cell binding domain comprises at least one sequence with a sequence identity of at least 90%, preferably at least 95%, more preferably at least 98% to SEQ ID No. 22 or 24 and/or SEQ ID No. 23 or 25, wherein the at least one sequence binds the antigen CD3.

Preferably, the humanized anti-human CD3 effector cell binding domain comprises a variable region of the heavy chain (V_{H}) according to SEQ ID No. 25 and a variable region of the light chain (V_{L}) according to SEQ ID No. 24, e.g. in the form of an scFv. Preferably, V_{H} and V_{L} are connected via glycine serine linker.

In some embodiments, the effector cell binding domain is a humanized anti-TCR domain and preferably comprises at least one of the following sequences:
i) a V_{L} according to SEQ ID No. 26 and/or
ii) a V_{H} according to SEQ ID No. 27.

In embodiments, the effector cell binding domain comprises at least one sequence with a sequence identity of at least 90%, preferably at least 95%, more preferably at least 98% to the sequences according to SEQ ID No. 26 or SEQ ID No. 27, wherein the at least one sequence binds the antigen TCR.

In some embodiments, the effector cell binding domain is a humanized anti-CD16A domain and preferably comprises at least one of the following sequences:
i) a V_{L} according to SEQ ID No. 28 and/or
ii) a V_{H} according to SEQ ID No. 29,
preferably, a V_{L} according to SEQ ID No. 28 and a V_{H} according to SEQ ID No. 29.

In an embodiment, the effector cell binding domain comprises at least one sequence with a sequence identity of at least 90%, preferably at least 95%, more preferably at least 98% to the sequences according to SEQ ID No. 28 or SEQ ID No. 29, wherein the at least one sequence binds the antigen CD16A.

In embodiments, the variable regions of the at least one PD-L1 and/or PD-L2 binding domain and/or the effector cell binding domain comprise a humanized amino acid sequence.

In embodiments, the different domains of the targeting module according to the invention are linked with each other by a linker. The linker comprises a short sequence of preferably 10 to 20 amino acid residues. In embodiments, the targeting module comprises a flexible peptide sequence that is selected such that the domains have a three-dimensional folding that allows them to exhibit the specificity for effector cell and target cell binding. Preferred linkers are glycine-serine linkers with the structure (GₓS_{y}) with x and y selected from 1 to 10, preferably 1 to 5. Mostly preferred are 1 to 10 repeats of the sequence G₄S₁. Moreover, linkers are preferred that are constituted of a peptide sequence that can increase the protease resistance of the antibody derivatives.

Preferably, the targeting module according to the invention is expressed as a fusion protein.

In embodiments, the targeting module according to the invention comprises a further domain selected from the group comprising co-stimulatory ligands, radionuclides, cell death inducing chemical compounds and half-life increasing domains, preferably IgG1 Fc, IgG2 Fc, IgG3 Fc, IgG4 Fc, HSA or mutants thereof. As used herein, the term “mutants” refers to proteins having at least 85% sequence identity to the half-life increasing domain, preferably at least 95% sequence identity. Advantageously, the mutant is capable of having one or more activities of the named peptides or proteins, in particular the mutant increases the half-life like the half-life increasing domain.

In preferred embodiments, the targeting module according to the invention comprises a further domain selected from the group comprising co-stimulatory ligands, radionuclides, cell death inducing chemical compounds and half-life increasing domains, preferably IgG1 Fc, IgG2 Fc, IgG3 Fc, IgG4 Fc or HSA.

In embodiments, the length of the targeting module according to the invention is in the range of 20 to 1600 amino acids.

In some embodiments, the targeting module according to the invention is used in a method for stimulating a chimeric antigen receptor mediated immune response in a mammal, wherein the targeting module is administered in combination with a vector or a cell comprising a nucleotide sequence encoding a switchable chimeric antigen receptor and at least one further targeting module, wherein the at least one further targeting module comprises at least one target cell binding domain and a tag-binding domain or a tag, wherein the at least one target cell binding domain is an antibody, an antibody fragment, a protein, a peptide or a low molecular weight organic ligand that binds to surface antigens selected from the group comprising CD2, CD3, CD4, CD8, CD10, CD19, CD20, CD22, CD23, CD25, CD30, CD33, CD38, CD44, CD44v6 CD52, CD90, CD99, CD123, CD133, CD150 CD181, CD182, CD184, CD223, CD229, CD269 (BCMA), CD273, CD274, CD276, CD279, CD319, CD366 and CD371, interleukin receptors, especially preferred IL-8Rα (CXCR1), IL-8Rβ (CXCR2), IL-11Rα, IL-11Rβ, IL-13Rα1 and 2, CXCR4, c-Met, mesothelin, members of the epidermal growth factor receptor family and mutants thereof, especially preferred ErbB1, ErbB2, ErbB3, ErbB4 or mutants thereof, members of the tumor necrosis factor receptor superfamily, ephrins, ephrin receptors, especially preferred EphA1-10, EphA5 or EphB1-6, prostate specific antigens prostate stem cell antigen (PSCA) and prostate specific membrane antigen (PSMA), embryonic antigens carcinoembryonic antigen (CEA) and fetal acethylcholine receptor, members of the vascular endothelia growth factor family, epithelia cell adhesion molecule EpCAM, alphafetoprotein AFP, members of the intercellular adhesion molecule family, members of the mucin protein family, follicle stimulating hormone receptor (FSHR), the human high molecular weight-melanoma-associated antigen (HMW-MAA), folate binding protein FBP, folate receptors, somatostatin receptors, ligands of the NKG2D receptor, cytokine receptors, members of the epithelia glycoprotein family, diasialogangliosides, glypicans, G protein-coupled receptors, members of the carbonic anhydrase family, members of the carbohydrate antigen family, Notch ligands, melanoma-associated chondroitin sulfate proteoglycan (MCSP), glycoprotein A33, guanylate cyclase 2C and tumor-specific glycans, including mutants and analogues of the named antibodies, antibody fragments, proteins, peptides or low molecular weight organic ligands, wherein the targeting module according to the invention and the at least one further targeting module comprise different target cell binding domains, and identical tag-binding domains or a tags.

As used herein, the term “target cell binding domain” refers to a peptide, protein, or low molecular weight organic ligand, which specifically binds a protein or protein complex (antigen) on the surface of a target cell, preferably a cancer cell, T cell, infected cell or pathogens or parasites.

As used herein, the terms “peptide”, “polypeptide” and “protein” are used interchangeably, and refer to a compound comprised of amino acid residues covalently linked by peptide bonds. A protein or peptide must contain at least two amino acids, and no limitation is placed on the maximum number of amino acids that can comprise a protein’s or peptide’s sequence.

As used herein, the term “low molecular weight organic ligand” refers to an organic molecule with a molecular weight of maximal 10 kilodalton, preferably of maximal 3 kilodalton, which specifically binds a protein or protein complex (antigen) on the surface of a target cell, preferably a cancer cell, T cell, infected cell or pathogens or parasites.

The term “target cell binding domain” also comprises soluble T cell receptors, which are composed of the alpha and beta or the gamma and delta chains of a T cell receptor (TCR), fragments or mutants thereof. Such TCR-derived binding moieties recognize and bind to peptides presented by human leukocyte antigen class (HLA) I and II protein complexes. Examples are, but are not limited to, TCRs specific for peptides derived from proteins like EGFR family, survivin, srylike high motility group box (SOX) protein family, melanoma-associated antigens (e.g. autoimmunogenic cancer/testis antigen NY-ESO-1, members of the melanoma antigen family A MAGEA, the preferentially expressed antigen in melanoma PRAME), and leukemia-associated antigens (e.g. Wilms tumor gene 1 WT1).

As used herein, the term “mutants” refers to peptides or proteins having at least 85% sequence identity to the named antibodies, antibody fragments, proteins or peptides, preferably at least 95% sequence identity. Advantageously, the mutants bind the identical antigens as the named antibodies, antibody fragments, proteins or peptides.

As used herein, the term “analogues” refers to molecules having a high degree of structural identity to the named antibodies, antibody fragments, proteins, peptides or low molecular weight organic ligands, preferably at least one atom, group of atoms, functional group or substructure is replaced with another group of atoms, e.g. a hydroxy group. In embodiments, an analogue of somatostatin (SRIF14) is octreotide or pasireotide. Advantageously, the analogues bind the identical antigens as the named antibodies, antibody fragments, proteins, peptides or low molecular weight organic ligands.

In embodiments, the analogues of the named antibodies, antibody fragments, proteins or peptides comprise modifications selected from the group comprising D amino acids, pseudo peptide bonds, aminoalcohols, non-proteinogenic amino acids, unnatural amino acids, amino acids with modified side chains and/or circular proteins. Advantageously, these analogues reveal increased stability.

In further embodiments, the target cell binding domain is soluble T cell receptor consisting of the alpha and beta or the gamma and delta chain of a T cell receptor (TCR).

In preferred embodiments, the at least one further targeting module comprises at least one target cell binding domain and a tag-binding domain or a tag, wherein the at least one target cell binding domain is an antibody or an antibody fragment that binds to surface antigens selected from the group comprising CD2, CD3, CD4, CD8, CD10, CD19, CD20, CD22, CD23, CD25, CD30, CD33, CD38, CD44, CD44v6 CD52, CD90, CD99, CD123, CD133, CD150 CD181, CD182, CD184, CD223, CD229, CD269 (BCMA), CD273, CD274, CD276, CD279, CD319, CD366 and CD371, interleukin receptors, especially preferred IL-8Rα (CXCR1), IL-8Rβ (CXCR2), IL-11Rα, IL-11Rβ, IL-13Rα1 and 2, CXCR4, c-Met, mesothelin, members of the epidermal growth factor receptor family, especially preferred ErbB1, ErbB2, ErbB3 or ErbB4; members of the tumor necrosis factor receptor superfamily, ephrins, ephrin receptors, especially preferred EphA1-10, EphA5 or EphB1-6, prostate specific antigens prostate stem cell antigen (PSCA) and prostate specific membrane antigen (PSMA), embryonic antigens carcinoembryonic antigen (CEA) and fetal acethylcholine receptor, members of the vascular endothelia growth factor family, epithelia cell adhesion molecule EpCAM, alphafetoprotein AFP, members of the intercellular adhesion molecule family, members of the mucin protein family, follicle stimulating hormone receptor (FSHR), the human high molecular weight-melanoma-associated antigen (HMW-MAA), folate binding protein FBP, folate receptors, somatostatin receptors, ligands of the NKG2D receptor, cytokine receptors, members of the epithelia glycoprotein family, diasialogangliosides, glypicans, G protein-coupled receptors, members of the carbonic anhydrase family, members of the carbohydrate antigen family, Notch ligands, melanoma-associated chondroitin sulfate proteoglycan (MCSP), glycoprotein A33, guanylate cyclase 2C and tumor-specific glycans, wherein the targeting module according to the invention and the at least one further targeting module comprise different target cell binding domains, and identical tag-binding domains or a tags.

In further embodiments, the at least one further targeting module comprises two target cell binding domains binding to surface antigens selected from PSCA and PSMA, CD19 and CD20, CD19 and CD22, CD19, CD20 and CD22, CD19 and CD123, CD33 and CD123, CD33 and CD99, CD33 and CD366, CD123 and CD366, CD123 and CD371, CD366 and CD371, ErbB-1 and ErbB-2, PSCA and ErbB-2, PSMA and CEA, IL-13Rα2 and ErbB-2, CD38 and CD269, mesothelin and mucin 16.

Preferably, the targeting module according to the invention comprises one of the sequences according to SEQ ID No. 57 to 59, more preferably the targeting module according to the invention consists of one of the sequences according to SEQ ID No. 57 to 59.

In preferred embodiments, the targeting module according to the invention is used in a method for stimulating a chimeric antigen receptor mediated immune response in a mammal, wherein the targeting module is administered, preferably in the range of 3 days to 30 days, after the administration of the vector or cell comprising a nucleotide sequence encoding a switchable chimeric antigen receptor and at least one further targeting module. Advantageously, by using the targeting module in combination with a further targeting module, wherein the targeting module is administered after the administration of the vector or cell comprising a nucleotide sequence encoding a switchable chimeric antigen receptor and the further targeting module, adverse systemic effects of the PD-L1 therapy are reduced. Furthermore, additional such doses of the targeting modules may be administered following resting periods to reactivate the switchable chimeric antigen receptor carrying effector cells.

More preferably, the targeting module according to the invention is used in a method for stimulating a chimeric antigen receptor mediated immune response in a mammal, wherein the at least one further targeting module is administered on its own, preferably one hour to 2 days, more preferably 4 to 24 hours, prior to the administration of the vector or cell comprising a nucleotide sequence encoding a switchable chimeric antigen receptor and the targeting module according to the invention is administered, preferably in the range of 3 days to 30 days, after the administration of the vector or cell comprising a nucleotide sequence encoding a switchable chimeric antigen receptor. Furthermore, additional such doses of the targeting module may be administered following resting periods to reactivate the switchable chimeric antigen receptor carrying effector cells.

The present invention further comprises a nucleic acid, a vector or a cell comprising a nucleotide sequence encoding a targeting module according to the invention for use in a method for stimulating a chimeric antigen receptor mediated immune response in a mammal, preferably for use in the treatment of cancer, infectious disease or autoimmune disease; wherein the nucleic acid, vector or cell is administered in combination with a vector or a cell comprising a nucleotide sequence encoding a switchable chimeric antigen receptor, wherein the switchable chimeric antigen receptor comprises three domains, wherein
the first domain is a tag-binding domain or a tag,
the second domain is an extracellular hinge and a transmembrane domain and
the third domain is a signal transduction domain,
wherein the tag-binding domain of the targeting module binds to the tag of the switchable chimeric antigen receptor or the tag of the targeting module binds to the tag-binding domain of the switchable chimeric antigen receptor.

According to the invention, the nucleic acid, vector and/or cell are isolated. As used herein, the term “isolated” means altered or removed from the natural state.

In embodiments, the nucleic acid is a cDNA. As used herein, the term “cDNA” (complementary DNA) refers to double-stranded DNA synthesized from a single stranded RNA, e.g. mRNA, in a reaction catalyzed by the enzyme reverse transcriptase. In embodiments, cDNA is of synthetic origin. In further embodiments, cDNA is derived from mRNA, therefore containing only exons but no introns, as opposed to genomic DNA.

The vector is preferably a plasmid, an artificial chromosome, linearized DNA or RNA, a virus particle or another vector that contains an expression cassette that is incorporated stably into the genome of a host cell or host organism.

In embodiments, the cell is selected from immune cells, preferably with cytolytic, phagocytic or immunosuppressive activity, such as T cells, Natural Killer (NK) cells and macrophages. In preferred embodiments, the cell is selected from T cells, including alpha/beta and gamma/delta T cells or subpopulations of T cells like stem-cell memory T cells or central memory T cells, cytotoxic T cells; or NK cells.

In embodiments, the nucleic acid, vector or cell further comprises an inducible expression system. In some embodiments, the inducible expression system is based on a prokaryotic operon, including, but not limited to, the lac operon, transposon Tn10 or tetracycline operon. In other embodiments, the inducible expression system is based on components of a eukaryotic signaling pathway, including, but not limited to, expression systems based on a steroid receptor, an estrogen receptor, progesterone or metallothionein.

In preferred embodiments, the inducible expression system is selected from Tet-On® (also rtTA; TET Systems, Takara Bio), Tet-On® Advanced (also rtTA2^{S}-M2 or rtTA2-syn1; TET Systems, Takara Bio), Tet-On® 3G (also rtTA-V16; TET Systems, Takara Bio), T-REx® (Life Technologies) or a promoter responsive to nuclear factor of activated T cells (NFAT).

As used herein, the term “Tet-On” refers to an inducible expression system comprising at least a tetracycline response element (TRE) and reverse tetracycline transactivator (rtTA) protein or variant thereof. As used herein, the term “reverse tetracycline transactivator (rtTA) protein” refers to a protein, which is capable of binding the operator (tetO) on the TRE, if bound by a tetracycline or a derivative of tetracycline, e.g. doxycycline. Thus, the introduction of tetracycline or a derivative of tetracycline to the system initiates the transcription of the genetic product. In embodiments, the inducible expression system induces the transcription of the nucleotide sequence encoding a switchable chimeric antigen receptor and/or a nucleotide sequence encoding a targeting module according to the invention, preferably the inducible expression system induces the transcription of the nucleotide sequence encoding a targeting module according to the invention.

As used herein, the term “variant” refers to a nucleotide sequence, peptide or protein, which is capable of performing one or more functions of the nucleotide or named amino acid sequence.

As used herein, the term “derivative” refers to a molecule having a high degree of structural identity to the molecule, preferably the same scaffold, wherein at least one atom, group of atoms, functional group or substructure is replaced with another atom, group of atoms functional group or substructure, e.g. a hydroxy group. Advantageously, the derivative is capable of having one or more activities of the named molecule.

Advantageously, the Tet-On system is preferred over Tet-Off for its faster responsiveness and its activation rather than repression by tetracycline or a derivative of tetracycline.

In embodiments, the rtTA protein is a fusion protein of rTetR (reverse tetracycline repressor), a mutated version of TetR (tetracycline repressor) from Escherichia coli, and the activation domain of virion protein 16 (VP16) from Herpes Simplex Virus. In preferred embodiments, rtTA comprises SEQ ID No. 30, SEQ ID No. 31 or SEQ ID No. 32.

As used herein, the term “tetracycline response element (TRE)” refers to at least one TetO sequence with a minimal promoter, responding to binding of the rtTA protein by increased expression of the gene or genes downstream of its promoter.

In embodiments, the TRE consists of 2 to 9 repeats, preferably 7 repeats, of the 19 bp bacterial TetO sequence according to SEQ ID No. 33, preferably separated by spacer sequences, more preferably by spacer sequences comprising SEQ ID No. 34.

In preferred embodiments, the TRE is SEQ ID No. 35.

As used herein, the term “Tet-On Advanced” refers to a Tet-On system with reduced basal expression and increased sensitivity to doxycycline, which is human codon optimized and comprises three minimal transcriptional activation domains, corresponding to the rtTA of SEQ ID No. 31.

As used herein, the term “Tet-On 3G” refers to a Tet-On system with further increased sensitivity to doxycycline, corresponding to the rtTA of SEQ ID No. 32.

As used herein, the term “T-REx” refers to a system, where the gene of interest is flanked by an upstream CMV promoter and two TetO2 sites. Binding of TetR homodimers to each TetO2 site represses gene expression in the absence of tetracycline or a derivative of tetracycline. In the presence of tetracycline or a derivative of tetracycline to the TetR homodimers, they dissociate from TetO2 resulting in expression of the gene of interest.

As used herein, the term “promoter responsive to nuclear factor of activated T cells (NFAT)” refers to a promoter naturally occurring in human cell nucleus, which is bound by nuclear factors of activated T cells (NFAT) initiating the transcription of the genetic product. As used herein, the term “nuclear factors of activated T cells (NFAT)” refers to transcription factors with one DNA binding domain for transcription and two calcineurin binding domains for the activation by calcineurin. The activation by calcineurin takes place by the activation of the serine/threonine phosphatase calcineurin by calmodulin, a calcium sensor protein. Activated calcineurin dephosphorylates the serine-rich region (SRR) and SP-repeats in the amino termini of NFAT proteins, resulting in a conformational change that exposes a nuclear localization signal, resulting in NFAT nuclear import.

In embodiments, the promoter responsive to NFAT consists of 2 to 12 repeats, preferably 6 repeats, of the 30 bp human NFAT binding sequence according to SEQ ID No. 36, optionally separated by spacer sequences, followed by a minimal promoter.

In embodiments, the minimal promoter is derived from the interleukin-2 (IL-2) promoter according to SEQ ID No. 37 or SEQ ID No. 38.

In embodiments, the promoter responsive to NFAT has SEQ ID No. 39 or SEQ ID No. 40.

In further embodiments, the nucleic acid, vector or cell according to the invention further comprises a nucleotide sequence encoding a switchable chimeric antigen receptor, wherein the switchable chimeric antigen receptor comprises three domains, wherein
the first domain is a tag-binding domain or tag,
the second domain is an extracellular hinge and a transmembrane domain and
the third domain is a signal transduction domain,
wherein the tag-binding domain of the targeting module binds to the tag of the switchable chimeric antigen receptor or the tag of the targeting module binds to the tag-binding domain of the switchable chimeric antigen receptor.

Another aspect of the invention is a nucleic acid, a vector or a cell comprising
an inducible expression system, preferably selected from Tet-On, Tet-On Advanced, Tet-On 3G, T-REx or a promoter responsive to NFAT, and
a nucleotide sequence encoding a switchable chimeric antigen receptor, wherein the switchable chimeric antigen receptor comprises three domains, wherein
   the first domain is a tag-binding domain or tag,
   the second domain is an extracellular hinge and a transmembrane domain and
   the third domain is a signal transduction domain, and
a nucleotide sequence encoding a targeting module according to the invention,
wherein the tag-binding domain of the targeting module binds to the tag of the switchable chimeric antigen receptor or the tag of the targeting module binds to the tag-binding domain of the switchable chimeric antigen receptor.

In embodiments, the inducible expression system induces the transcription of the nucleotide sequence encoding a switchable chimeric antigen receptor and/or a nucleotide sequence encoding a targeting module according to the invention, preferably the inducible expression system induces the transcription of the nucleotide sequence encoding a targeting module according to the invention.

The invention further comprises a pharmaceutical composition comprising the targeting module according to the invention, the nucleic acid, vector or cell according to the invention for use in a method for stimulating a chimeric antigen receptor mediated immune response in a mammal, preferably for use in the treatment of cancer, infectious disease or autoimmune disease; wherein the pharmaceutical composition is administered in combination with a vector or a cell comprising a nucleotide sequence encoding a switchable chimeric antigen receptor, wherein the switchable chimeric antigen receptor comprises three domains, wherein
the first domain is a tag-binding domain or a tag,
the second domain is an extracellular hinge and a transmembrane domain and
the third domain is a signal transduction domain,
wherein the tag-binding domain of the targeting module binds to the tag of the switchable chimeric antigen receptor or the tag of the targeting module binds to the tag-binding domain of the switchable chimeric antigen receptor.

The pharmaceutical composition is preferably administered parenterally, particularly preferred intravenously. In an embodiment, the pharmaceutical composition is present in a form suitable for intravenous administration. Preferably, the pharmaceutical composition is a solution, emulsion or suspension.

In embodiments, the pharmaceutical composition is an injectable buffered solution comprising a concentration in the range of 1 ng/ml to 500 mg/ml of the targeting module, the nucleic acid, vector and/or cell according to the invention, preferably in the range of 5 µg/ml to 5 mg/ml.

In embodiments, the pharmaceutical composition further comprises a pharmaceutically acceptable dilution agent or carrier. In further embodiments, the carrier is selected from water, an aqueous buffer solution, 0.9% saline solution, 5% glucose, 5% xylitol, 0.3% glycine solution, ringer solutions, amino acid solutions and a similar solvent. In further embodiments, the aqueous buffer solution is selected from an aqueous histidine, sodium succinate, sodium citrate, sodium phosphate or potassium phosphate buffered solution with a pH value in the range of pH 5.0 to pH 7.0. In embodiments, the buffer solution has a buffer concentration in the range of 1 mmol/l (mM) to 500 mM, preferably in the range of 5 mM to 20 mM, especially preferred in the range of 5 mM to 10 mM. In further embodiments, the carrier comprises sodium chloride, preferably in a concentration in the range of 1 mM to 300 mM, especially preferred 150 mM. In embodiments, the pharmaceutical composition further comprises a stabilizer, preferably in a concentration in the range of 1 mM to 900 mM, especially preferred in the range of 50 mM and 600 mM. In embodiments, the stabilizer is sucrose, trehalose or L-methionine.

In some embodiments, the pharmaceutical composition further comprises pharmaceutically acceptable excipients. The term “pharmaceutically acceptable excipients” refers to compounds, which provide approximately physiological conditions and/or increase the stability, such as agents for adjusting the pH value and buffering agents, agents for adjusting the toxicity and the like. In embodiments, pharmaceutically acceptable excipients are selected from sodium acetate, sodium chloride, potassium chloride, calcium chloride, sodium lactate and polysorbate-80, preferably polysorbate-80 in the range of 0.0001% (w/v) to 1% (w/v), especially preferred in the range of 0.001% (w/v) to 0.1% (w/v).

In a preferred embodiment, the pharmaceutical composition comprises the targeting module in a dosage quantity in the range of 25 µg/day to 100 mg/day, preferably dosage quantities in the range of 0.1 mg/day to 20 mg/day.

In further embodiments, the pharmaceutical composition is sterile. The pharmaceutical composition is sterilized by conventional well-known techniques including, but not limited to, sterile filtration.

In embodiments, the pharmaceutical composition is used for administration to a subject.

In embodiments, the pharmaceutical composition is lyophilized prior to storage or stored as solution at ambient temperature or below, including, but not limited to, frozen storage.

In embodiments, the pharmaceutical composition is reconstituted and/or diluted in an infusion and stabilizer solution prior to administration to a subject. The solutions used for reconstitution or infusion/stabilization may contain any of the components mentioned for the pharmaceutical composition or similar components.

Another aspect of the invention is a kit comprising
a) a targeting module according to the invention, the nucleic acid, vector or cell according to the invention and
b) a vector or a cell comprising a nucleotide sequence encoding a switchable chimeric antigen receptor,
wherein the switchable chimeric antigen receptor comprises three domains, wherein
   the first domain is a tag-binding domain or tag,
   the second domain is an extracellular hinge and a transmembrane domain and
   the third domain is a signal transduction domain,
wherein the tag-binding domain of the targeting module binds to the tag of the switchable chimeric antigen receptor or the tag of the targeting module binds to the tag-binding domain of the switchable chimeric antigen receptor.

In embodiments, the tag is a peptide epitope tag. In further embodiments, the tag comprises 10 to 20 amino acids.

In embodiments, the peptide epitope tag is a myc-tag, a His-tag, a short linear peptide sequence from yeast transcription factor GCN4, preferably according to SEQ ID No. 10, SEQ ID No. 11 or mutants thereof; a leucine zipper sequence, preferably SYNZIP 1 to SYNZIP 48, BATF, FOS, ATF4, ATF3, BACH1, JUND, NFE2L3, HEPTAD (Reinke et al. 2010), a sequence according to SEQ ID No. 12 or SEQ ID No. 13 or mutants thereof, or a short linear peptide sequence from a human nuclear protein, preferably from the human La protein. Preferably, the peptide epitope tag from the human La protein is the human La epitope E5B9 according to SEQ ID No. 14 or E7B6 according to SEQ ID No. 15 or SEQ ID No. 16, most preferably the human La epitope E5B9 according to SEQ ID No. 14 or E7B6 according to SEQ ID No. 16.

In embodiments, the His-tag is an amino acid sequence consisting of histidine residues, preferably in the range of six to fourteen histidine residues.

In preferred embodiments, the peptide epitope tag is a myc-tag, a His-tag, a short linear peptide sequence from yeast transcription factor GCN4, preferably according to SEQ ID No. 10 or SEQ ID No. 11; a leucine zipper sequence, preferably SYNZIP 1 to SYNZIP 48, BATF, FOS, ATF4, ATF3, BACH1, JUND, NFE2L3, HEPTAD (Reinke et al. 2010), a sequence according toSEQ ID No. 12 or SEQ ID No. 13; or a short linear peptide sequence from a human nuclear protein, preferably from the human La protein.

In further embodiments, the tag-binding domain is an antibody or an antibody fragment.

In embodiments, the tag-binding domain is an antibody or an antibody fragment binding to a myc-tag, a His-tag, a short linear peptide sequence from yeast transcription factor GCN4, a leucine zipper sequence or a short linear peptide sequence from a human nuclear protein.

In embodiments, the tag-binding domain binds to a tag from the human nuclear La protein, preferably the tag-binding domain is an antibody or an antigen-binding fragment, wherein the tag-binding domain constitutes an anti-La epitope scFv, more preferably an anti-La epitope scFv comprising a V_{L} according to the following sequence:

DIVMTQSPDSLAVSLGERATINCX¹SSQSLLNSRTX²KNYLAWYQQ
KPGQPPKLLIYWASTRX³SGVPDRFSGSGSGTDFTLTISSLQAEDVAV
YYCKQSYNLX⁴TFGGGTKVEIIK(SEQ ID No. 17),

wherein X¹ to X⁴ are variable amino acid residues, preferably selected from a proteinogenic alpha amino acid residue; or a sequence having at least 85% sequence identity, preferably at least 95% sequence identity; to one of the sequences SEQ ID No. 19 or SEQ ID No. 21.

In some embodiments, the variable amino acid residues X¹ to X⁴are selected as follows:
X¹ is selected from polar and/or positive charged residues, such as Serine, Threonine, Asparagine, Glutamine, Histidine, Lysine and Arginine; preferably X¹ is Lysine or Arginine;
X² is preferably selected from Lysine and Proline;
X³ is selected from polar and charged residues, such as Asparagine, Aspartic Acid, Glutamine, Glutamic acid, Histidine, Lysine and Arginine, preferably Glutamic acid and Lysine;
X⁴ is selected from hydrophobic residues, such as Isoleucine, Leucine, Valine, Alanine, Methionine, Phenylalanine, Proline and Tryptophan; preferably Z⁴ is selected from Leucine and Proline.

In further embodiments, the tag-binding domain is an antibody or an antigen-binding fragment, wherein the tag-binding domain constitutes an anti-La epitope scFv comprising a sequence having at least 85% sequence identity, preferably at least 95% sequence identity; to one of the sequences SEQ ID No. 18 or SEQ ID No. 20.

Preferably, the tag-binding domain constitutes an anti-La epitope scFv comprising a sequence having each at least 85% sequence identity, preferably at least 95% sequence identity; to the sequences according to SEQ ID NO. 18 (V_{H}) and SEQ ID NO. 19 (V_{L}) or the sequences according to SEQ ID NO. 20 (V_{H}) and SEQ ID NO. 21 (V_{L}).

Most preferably, the tag-binding domain constitutes the anti-La 5B9 scFv according to SEQ ID NO. 18 (V_{H}) and SEQ ID NO. 19 (V_{L}) or the anti-La 7B6 scFv according to SEQ ID NO. 20 (V_{H}) and SEQ ID NO. 21 (V_{L}).

As used herein, the term “extracellular hinge and a transmembrane domain” refers to a flexible peptide sequence connected to the tag-binding domain or tag, which anchors the switchable CAR into the cell membrane of the cell and protruding from the surface of the cell for optimal binding to its particular targeting module.

In embodiments, the extracellular hinge and transmembrane domain is selected from hinge and transmembrane domains of human CD28 molecule, CD8a chain, NK cell receptors, preferably natural killer group NKG2D; or parts of the constant region of an antibody and combinations thereof. As used herein, the term “combinations thereof” refers to combinations of different hinge and transmembrane domains.

Pinthus et al. 2003, Pinthus et al. 2004 and Cartellieri et al. 2016 describe the use of hinge and transmembrane domains of the human CD28 molecule in CARs (Pinthus et al. 2003, Pinthus et al. 2004, Cartellieri et al. 2016).

Carpentino et al., Milone et al. and Zhao et al. describe the use of hinge and transmembrane domains of human CD8α molecule in CARs (Carpentino et al. 2009, Milone et al. 2009, Zhao et al. 2009).

Zhang et al. 2005 and Zhang et al. 2006 describe the use of hinge and transmembrane domains of NKG2D in CARs (Zhang et al. 2005, Zhang et al. 2006).

Hombach et al., Frigault et al. and Wang et al. describe the use of hinge and transmembrane domains of parts of the constant region of immunoblobulin G1 (IgG) (Hombach et al. 2007, Frigault et al. 2015, Wang et al. 2007). Frigault et al. describes the use of hinge domains of the constant region of IgG4.

Examples of combinations of the extracellular hinge and transmembrane domain are, but are not limited to, CD28 extracellular hinge and transmembrane domain, CD8alpha extracellular hinge and transmembrane domain, IgG1 or IgG4 constant regions combined with CD28 or CD137 transmembrane domain.

As used herein, the term “signal transduction domain” refers to a peptide sequence which transmits a signal into the cell by cross-linkage of the cell expressing the switchable CAR (effector cell) to a human cell surface protein or protein complex (target cell). Cross-linkage between effector and target cell is mediated by the targeting module according to the invention.

In embodiments, the signal transduction domain is selected from cytoplasmic regions of CD28, CD137 (4-1BB), CD134 (OX40), CD278 (ICOS), DAP10 and CD27, programmed cell death-1 (PD-1), cytotoxic T-lymphocyte antigen 4 (CTLA-4), cytoplasmic regions of CD3 chains, DAP12, CD122 (interleukin-2 receptor β), CD132 (interleukin-2 receptor γ), CD127 (interleukin-7 receptor α), CD360 (interleukin-21 receptor) activating Fc receptors and mutants thereof.

As used herein, the term “mutants” refers to proteins having at least 85% sequence identity to the signal transduction domains, preferably at least 95% sequence identity. Advantageously, the mutant transmits a signal into the cell by cross-linkage of the cell expressing the switchable CAR (effector cell) to a human cell surface protein or protein complex (target cell) in the same way as the named signal transduction domains.

In embodiments, mutants are truncated versions. As used herein, the term “truncated versions” refers to shortened proteins having at least 85% sequence identity to the signal transduction domains, preferably at least 95% sequence identity, more preferably having a chain length of at least 85% and a sequence identity of 100%, most preferably a chain length of at least 95% and a sequence identity of 100%. Advantageously, the truncated version has an activity of at least 80%, preferably of at least 90%, more preferably of at least 95%; of the named signal transduction domains.

Hombach et al., Maher et al. and Cartellieri et al. describe the use of cytoplasmic regions of CD28 as signal transduction domain in CARs (Hombach et al. 2001, Maher et al. 2002, Cartellieri et al. 2016). Guedan et al. describes the use of a mutant of cytoplasmic regions of CD28 as signal transduction domain (Guedan et al. 2020).

Milone et al. and Finney et al. describe the use of cytoplasmic regions of CD137 (4-1BB) as signal transduction domain (Finney et al. 2004, Milone et al. 2009).

Finney et al. and Hombach and Abken describe the use of cytoplasmic regions of CD134 (OX40) as signal transduction domain in CARs (Finneyet al. 2004, Hombach and Abken 2011, Hombach and Abken 2013).

Guedan et al. describes the use of cytoplasmic regions of CD278 (ICOS) as signal transduction domain (Guedan et al. 2018).

Zhang et al. describes the use of DAP10 as signal transduction domain (Zhang et al. 2005).

Fedorov et al. describes the use of programmed cell death 1 (PD-1) and of cytotoxic T-lymphocyte antigen 4 (CTLA-4) as signal transduction domain in CARs (Fedorovet al. 2013).

Gong et al. and Gade et al. describe the use of cytoplasmic regions CD3 chains, in particular the CD3ζ chain, as signal transduction domain in CARs (Gong et al. 1999, Gade et al. 2005).

Töpfer et al. describes the use of DAP12 as signal transduction domain in CARs (Töpferet al. 2015).

Kagoya et al. describes the use of signaling chains or motifs derived from interleukin receptors as signal transduction domain in CARs (Kagoya et al. 2018).

Lamers et al. and Kershaw et al. describe the use of activating Fc receptors, in particular the Fc epsilon receptor y chain, as signal transduction domain (Lamers et al. 2004, Kershaw et al. 2006).

In preferred embodiments, the signal transduction domain is selected from cytoplasmic regions of CD28, CD137 (4-1BB), CD134 (OX40), CD278 (ICOS), DAP10 and CD27, programmed cell death-1 (PD-1), cytotoxic T-lymphocyte antigen 4 (CTLA-4), cytoplasmic regions of CD3 chains, DAP12, CD122 (interleukin-2 receptor β), CD132 (interleukin-2 receptor γ), CD127 (interleukin-7 receptor α) and CD360 (interleukin-21 receptor) activating Fc receptors.

In embodiments, the switchable chimeric antigen receptor comprises at least one signal transduction domain, preferably two, three, four or more signal transduction domains, especially preferably selected from cytoplasmic regions of CD28, CD137 (4-1BB), CD134 (OX40), CD278 (ICOS), DAP10 and CD27, programmed cell death-1 (PD-1), cytotoxic T-lymphocyte antigen 4 (CTLA-4), cytoplasmic regions of CD3 chains, DAP12, CD122 (interleukin-2 receptor β), CD132 (interleukin-2 receptor γ), CD127 (interleukin-7 receptor α), CD360 (interleukin-21 receptor), activating Fc receptors and mutants thereof.

In preferred embodiments, the switchable chimeric antigen receptor comprises at least one signal transduction domain, preferably two, three, four or more signal transduction domains, especially preferably selected from cytoplasmic regions of CD28, CD137 (4-1BB), CD134 (OX40), CD278 (ICOS), DAP10 and CD27, programmed cell death-1 (PD-1), cytotoxic T-lymphocyte antigen 4 (CTLA-4), cytoplasmic regions of CD3 chains, DAP12, CD122 (interleukin-2 receptor β), CD132 (interleukin-2 receptor γ), CD127 (interleukin-7 receptor α) and CD360 (interleukin-21 receptor), activating Fc receptors.

In further embodiments, the switchable chimeric antigen receptor comprises a fourth domain, wherein the fourth domain is a short peptide linker in the extracellular portion of the receptor that may serve to detect the chimeric antigen receptor on the cell surface or stimulate the chimeric antigen receptor T cell.

In further embodiments, the fourth domain forms a linear epitope for a monoclonal antibody (mab) specifically binding to the fourth domain. In some embodiments, the fourth domain comprises at least one linear epitope, preferably E7B6 according to SEQ ID No. 15 or SEQ ID No. 16.

In some embodiments, the fourth domain is located in between the tag-binding domain or the tag and the extracellular hinge domain or an integral part of the extracellular hinge domain.

Advantageously, the switchable CAR engrafted cells with the fourth domain can be specifically stimulated to proliferate preferentially and persist longer compared to non-engrafted cells either in vitro or in vivo. Further advantageously, the fourth domain may be also used to purify switchable CAR engrafted cells from mixed cell populations or to dampen switchable CAR engrafted cell mediated immune response and to eliminate switchable CAR engrafted cells in vivo.

In further embodiments, the switchable CAR comprises a signal peptide. Advantageously, the signal peptide allows for expression on the cell surface of an effector cell. In an embodiment, the signal peptide is at the N-terminus of the switchable CAR nucleotide sequence in front of the tag-binding domain or the tag. In some embodiments, the signal peptide targets proteins to the secretory pathway either co-translationally or post-translationally and is selected from leader peptides from proteins like CD28, CD8alpha, IL-2 or the heavy or light chains of antibodies of human origin to avoid immunogenic reactions.

In further embodiments, the nucleic acid is a cDNA.

In embodiments, the nucleic acid encodes a switchable CAR according one of the sequences SEQ ID NO. 41 to 48. Preferably, the nucleic acid is one of the sequences SEQ ID NO. 49 to 56.

In some embodiments, the cell comprising a nucleotide sequence encoding a switchablechimeric antigen receptor is selected from immune cells, preferably with cytolytic, phagocytic or immunosuppressive activity, such as T cells, Natural Killer (NK) cells and macrophages. In preferred embodiments, the cell is selected from T cells, including alpha/beta and gamma/delta T cells or subpopulations of T cells like stem-cell memory T cells or central memory T cells, cytotoxic T cells; or NK cells.

In embodiments, the kit further comprises at least one further targeting module, wherein the at least one further targeting module comprises at least one target cell binding domain and a tag-binding domain or a tag, wherein the at least one target cell binding domain is an antibody, an antibody fragment, a protein, a peptide or a low molecular weight organic ligand that binds to surface antigens selected from the group comprising CD2, CD3, CD4, CD8, CD10, CD19, CD20, CD22, CD23, CD25, CD30, CD33, CD38, CD44, CD44v6 CD52, CD90, CD99, CD123, CD133, CD150 CD181, CD182, CD184, CD223, CD229, CD269 (BCMA), CD273, CD274, CD276, CD279, CD319, CD366 and CD371, interleukin receptors, especially preferred IL-8Rα (CXCR1), IL-8Rβ (CXCR2), IL-11Rα, IL-11Rβ, IL-13Rα1 and 2, CXCR4, c-Met, mesothelin, members of the epidermal growth factor receptor family and mutants thereof, especially preferred ErbB1, ErbB2, ErbB3, ErbB4 or mutants thereof, members of the tumor necrosis factor receptor superfamily, ephrins, ephrin receptors, especially preferred EphA1-10, EphA5 or EphB1-6, prostate specific antigens prostate stem cell antigen (PSCA) and prostate specific membrane antigen (PSMA), embryonic antigens carcinoembryonic antigen (CEA) and fetal acethylcholine receptor, members of the vascular endothelia growth factor family, epithelia cell adhesion molecule EpCAM, alphafetoprotein AFP, members of the intercellular adhesion molecule family, members of the mucin protein family, follicle stimulating hormone receptor (FSHR), the human high molecular weight-melanoma-associated antigen (HMW-MAA), folate binding protein FBP, folate receptors, somatostatin receptors, ligands of the NKG2D receptor, cytokine receptors, members of the epithelia glycoprotein family, diasialogangliosides, glypicans, G protein-coupled receptors, members of the carbonic anhydrase family, members of the carbohydrate antigen family, Notch ligands, melanoma-associated chondroitin sulfate proteoglycan (MCSP), glycoprotein A33, guanylate cyclase 2C and tumor-specific glycans, including mutants and analogues of the named antibodies, antibody fragments, proteins, peptides or low molecular weight organic ligands, wherein the targeting module according to the invention and the at least one further targeting module comprise different target cell binding domains, and identical tag-binding domains or tags. Such kits are suitable to avoid tumor escape from the immune system.

The term “target cell binding domain” also comprises soluble T cell receptors, which are composed of the alpha and beta or the gamma and delta chains of a T cell receptor (TCR), fragments or mutants thereof.

In preferred embodiments, the kit further comprises at least one further targeting module, wherein the at least one further targeting module comprises at least one target cell binding domain and a tag-binding domain or a tag, wherein the at least one target cell binding domain is an antibody or an antibody fragment that binds to surface antigens selected from the group comprising CD2, CD3, CD4, CD8, CD10, CD19, CD20, CD22, CD23, CD25, CD30, CD33, CD38, CD44, CD44v6 CD52, CD90, CD99, CD123, CD133, CD150 CD181, CD182, CD184, CD223, CD229, CD269 (BCMA), CD273, CD274, CD276, CD279, CD319, CD366 and CD371, interleukin receptors, especially preferred IL-8Rα (CXCR1), IL-8Rβ (CXCR2), IL-11Rα, IL-11Rβ, IL-13Rα1 and 2, CXCR4, c-Met, mesothelin, members of the epidermal growth factor receptor family, especially preferred ErbB1, ErbB2, ErbB3 or ErbB4; members of the tumor necrosis factor receptor superfamily, ephrins, ephrin receptors, especially preferred EphA1-10, EphA5 or EphB1-6, prostate specific antigens prostate stem cell antigen (PSCA) and prostate specific membrane antigen (PSMA), embryonic antigens carcinoembryonic antigen (CEA) and fetal acethylcholine receptor, members of the vascular endothelia growth factor family, epithelia cell adhesion molecule EpCAM, alphafetoprotein AFP, members of the intercellular adhesion molecule family, members of the mucin protein family, follicle stimulating hormone receptor (FSHR), the human high molecular weight-melanoma-associated antigen (HMW-MAA), folate binding protein FBP, folate receptors, somatostatin receptors, ligands of the NKG2D receptor, cytokine receptors, members of the epithelia glycoprotein family, diasialogangliosides, glypicans, G protein-coupled receptors, members of the carbonic anhydrase family, members of the carbohydrate antigen family, Notch ligands, melanoma-associated chondroitin sulfate proteoglycan (MCSP), glycoprotein A33, guanylate cyclase 2C and tumor-specific glycans, wherein the targeting module according to the invention and the at least one further targeting module comprise differenttarget cell binding domains, and identical tag-binding domains or tags.

In embodiments, the kit comprises
a nucleic acid, vector or cell according to the invention and
a vector or a cell comprising a nucleotide sequence encoding a switchable chimeric antigen receptor,
wherein the switchable chimeric antigen receptor comprises three domains, wherein
   the first domain is a tag-binding domain or tag,
   the second domain is an extracellular hinge and a transmembrane domain and
   the third domain is a signal transduction domain,
at least one further targeting module,
wherein the tag-binding domain of the targeting module binds to the tag of the switchable chimeric antigen receptor or the tag of the targeting module binds to the tag-binding domain of the switchable chimeric antigen receptor, wherein the targeting module according to the invention and the at least one further targeting module comprise different target cell binding domains, and identical tag-binding domains or tags.

In embodiments, the kit comprises one to three targeting modules, preferably one targeting module according to the invention and one or two further targeting modules.

In embodiments, the tag-binding domain or tag is present at the amino terminal end of the polypeptide that comprises the switchable CAR. Advantageously, locating the tag-binding domain or tag at the amino terminus permits the tag-binding domain or tag unhampered access to the targeting module that is bound to the target cell.

In embodiments, the kit further comprises an inducer, preferably tetracycline or a derivative of tetracycline, e.g. doxycycline.

In embodiments of the kit, the targeting module, the vector and/or the cell comprising a nucleotide sequence encoding a switchable chimeric antigen receptor are in the form of a pharmaceutical composition.

The pharmaceutical composition is preferably administered parenterally, particularly preferred intravenously. In embodiments, the pharmaceutical composition is present in a form suitable for intravenous administration. Preferably, the pharmaceutical composition is a solution, emulsion or suspension.

In embodiments, the pharmaceutical composition is an injectable buffered solution comprising between 1 µg/ml to 500 mg/ml of the targeting module and/or the nucleic acid and/or the vector and/or the cell according to the invention, preferably between 50 µg/ml to 50 mg/ml.

In embodiments, the pharmaceutical composition is an injectable buffered solution comprising between 1,000 ml⁻¹ to 5·10⁸ ml⁻¹ of the cell comprising a nucleotide sequence encoding a switchable chimeric antigen receptor, preferably between 1·10⁵ ml⁻¹ to 2.5·10⁷ ml⁻¹.

In embodiments, the pharmaceutical composition further comprises a pharmaceutically acceptable dilution agent or carrier. In further embodiments, the carrier is selected from water, an aqueous buffer solution, 0.9% saline solution, 5% glucose, 5% xylitol, 0.3% glycine solution, ringer solutions, amino acid solutions and a similar solvent. In further embodiments, the aqueous buffer solution is selected from an aqueous histidine, sodium succinate, sodium citrate, sodium phosphate or potassium phosphate buffered solution with a pH value in the range of pH 5.0 to pH 8.0, preferably with a pH value in the range of pH 6.0 to pH 7.0. In embodiments, the buffer solution has a buffer concentration in the range of 1 mmol/l (mM) to 500 mM, preferably in the range of 5 mM to 20 mM, especially preferred in the range of 5 mM to 10 mM. In further embodiments, the carrier comprises sodium chloride, preferably in a concentration in the range of 0 mM to 300 mM, especially preferred 150 mM. In embodiments, the pharmaceutical composition further comprises a stabilizer, preferably in a concentration in the range of 1 mM to 900 mM, especially preferred in the range of 10 mM and 600 mM. In embodiments, the stabilizer is sucrose, trehalose or L-methionine.

In further embodiments, the pharmaceutical composition further comprises pharmaceutically acceptable excipients. The term “pharmaceutically acceptable excipients” refers to compounds, which provide approximately physiological conditions and/or to increase the stability, such as agents for adjusting the pH value and buffering agents, agents for adjusting the toxicity and the like or agents to enable frozen storage of the pharmaceutical composition In embodiments, pharmaceutically acceptable excipients are selected from sodium acetate, sodium chloride, potassium chloride, calcium chloride, sodium lactate and polysorbate-80, preferably polysorbate-80 in the range of 0.0001 % (w/v) to 1% (w/v), especially preferred in the range of 0.001% (w/v) to 0.1% (w/v).

In preferred embodiments, the pharmaceutical composition comprises the targeting module in a dosage quantity in the range of 25 µg/day to 100 mg/day, preferably dosage quantities in the range of 0.1 mg/day to 10 mg/day.

In further embodiments, the pharmaceutical composition is sterile. The pharmaceutical composition is sterilized by conventional well-known techniques including, but not limited, to sterile filtration.

In embodiments, the pharmaceutical composition is used for administration to a subject.

In embodiments, the pharmaceutical composition is lyophilized prior to storage or stored as solution at ambient temperature or below, including, but not limited to, frozen storage.

In embodiments, the pharmaceutical composition is reconstituted and/or diluted in an infusion and stabilizer solution prior to administration to a subject. The solutions used for reconstitution or infusion/stabilization may contain any of the components mentioned for the pharmaceutical composition or similar components.

In preferred embodiments, the pharmaceutical composition comprises the cell comprising a nucleotide sequence encoding a switchable chimeric antigen receptor in a dosage quantity in the range of 1·10⁷ to 1·10⁹ per administration, preferably dosage quantities in the range of 1·10⁸ to 5·10⁸ per administration.

Another aspect of the invention is the kit according to invention for use in a method for stimulating a chimeric antigen receptor mediated immune response in a mammal, preferably for use in the treatment of cancer, infectious disease or autoimmune disease.

In embodiments, the targeting module according to the invention, the nucleic acid, vector or cell according to the invention or the kit according to the invention is used for preparing a medication for therapeutic and/or diagnostic use in case of cancer, an infection or an autoimmune disease.

A further aspect of the invention is a method for stimulating a chimeric antigen receptor mediated immune response in a mammal, preferably a human, having cancer, an infectious or an autoimmune disease by administration of a targeting module, a cell or a vector, a pharmaceutical composition or a kit according to the invention. For therapeutic applications, a sterile pharmaceutical composition according to the invention or a sterile kit according to the invention, comprising a pharmacologically effective quantity of targeting module according to the invention and a vector or a cell comprising a nucleotide sequence encoding a switchable chimeric antigen receptor, is administered to a subject in order to treat the aforementioned illnesses.

In embodiments, the method for stimulating a chimeric antigen receptor mediated immune response in a mammal; preferably the method for treatment of cancer, infectious or autoimmune disease, further comprises the administration of an inducer, preferably tetracycline or a derivative of tetracycline, e.g. doxycycline.

In embodiments, an inducer, preferably tetracycline or a derivative of tetracycline, e.g. doxycycline; is administered orally or intravenously following administration of the nucleic acid, vector or cell according the invention.

In some embodiments, the method for stimulating a chimeric antigen receptor mediated immune response in a mammal, preferably the method for treatment of cancer, infectious or autoimmune disease, comprises the following steps:
a) administering to a mammal an effective amount of a targeting module according to the invention and
b) administering to the mammal an effective amount of a vector or a cell comprising a nucleotide sequence encoding a switchable chimeric antigen receptor,
wherein the switchable chimeric antigen receptor comprises three domains, wherein
   the first domain is a tag-binding domain or tag,
   the second domain is an extracellular hinge and a transmembrane domain and
   the third domain is a signal transduction domain,
wherein the tag-binding domain of the targeting module binds to the tag of the switchable chimeric antigen receptor or the tag of the targeting module binds to the tag-binding domain of the switchable chimeric antigen receptor,
wherein the targeting module is administered to a mammal prior to, concurrent with or after the administration of the vector or cell.

In further embodiments, the method for stimulating a chimeric antigen receptor mediated immune response in a mammal, preferably the method for treatment of cancer, infectious or autoimmune disease, comprises the following steps:
a) administering to a mammal an effective amount of a targeting module comprising at least one target cell binding domain and a tag-binding domain or a tag, wherein the at least one target cell binding domain is an antibody, antibody fragment, a protein, a peptide or a low molecular weight organic ligand that binds to surface antigens selected from the group comprising CD2, CD3, CD4, CD8, CD10, CD19, CD20, CD22, CD23, CD25, CD30, CD33, CD38, CD44, CD44v6 CD52, CD90, CD99, CD123, CD133, CD150 CD181, CD182, CD184, CD223, CD229, CD269 (BCMA), CD273, CD274, CD276, CD279, CD319, CD366 and CD371, interleukin receptors, especially preferred IL-8Rα (CXCR1), IL-8Rβ (CXCR2), IL-11Rα, IL-11Rβ, IL-13Rα1 and 2, CXCR4, c-Met, mesothelin, members of the epidermal growth factor receptor family and mutants thereof, especially preferred ErbB1, ErbB2, ErbB3, ErbB4 or mutants thereof, members of the tumor necrosis factor receptor superfamily, ephrins, ephrin receptors, especially preferred EphA1-10, EphA5 or EphB1-6, prostate specific antigens prostate stem cell antigen (PSCA) and prostate specific membrane antigen (PSMA), embryonic antigens carcinoembryonic antigen (CEA) and fetal acethylcholine receptor, members of the vascular endothelia growth factor family, epithelia cell adhesion molecule EpCAM, alphafetoprotein AFP, members of the intercellular adhesion molecule family, members of the mucin protein family, follicle stimulating hormone receptor (FSHR), the human high molecular weight-melanoma-associated antigen (HMW-MAA), folate binding protein FBP, folate receptors, somatostatin receptors, ligands of the NKG2D receptor, cytokine receptors, members of the epithelia glycoprotein family, diasialogangliosides, glypicans, G protein-coupled receptors, members of the carbonic anhydrase family, members of the carbohydrate antigen family, Notch ligands, melanoma-associated chondroitin sulfate proteoglycan (MCSP), glycoprotein A33, guanylate cyclase 2C and tumor-specific glycans, including mutants and analogues of the named antibodies, antibody fragments, proteins, peptides or low molecular weight organic ligands, and
b) administering to the mammal an effective amount of a vector or a cell comprising a nucleotide sequence encoding a switchable chimeric antigen receptor,
wherein the switchable chimeric antigen receptor comprises three domains, wherein
   the first domain is a tag-binding domain or tag,
   the second domain is an extracellular hinge and a transmembrane domain and
   the third domain is a signal transduction domain,
wherein the tag-binding domain of the targeting module binds to the tag of the switchable chimeric antigen receptor or the tag of the targeting module binds to the tag-binding domain of the switchable chimeric antigen receptor, and
c) administering to a mammal an effective amount of a targeting module according to the invention,
wherein the targeting module according to the invention and the at least one further targeting module comprise different target cell binding domains, and identical tag-binding domains or tags, wherein the method is carried out in the order of the steps a), b) and c).

The term “target cell binding domain” also comprises soluble T cell receptors, which are composed of the alpha and beta or the gamma and delta chains of a T cell receptor (TCR), fragments or mutants thereof.

In preferred embodiments, the method for stimulating a chimeric antigen receptor mediated immune response in a mammal, preferably the method for treatment of cancer, infectious or autoimmune disease, comprises the following steps:
a) administering to a mammal an effective amount of a targeting module comprising at least one target cell binding domain and a tag-binding domain or a tag, wherein the at least one target cell binding domain is an antibody, antibody fragment, a protein, a peptide or a low molecular weight organic ligand that binds to surface antigens selected from the group comprising CD2, CD3, CD4, CD8, CD10, CD19, CD20, CD22, CD23, CD25, CD30, CD33, CD38, CD44, CD44v6 CD52, CD90, CD99, CD123, CD133, CD150 CD181, CD182, CD184, CD223, CD229, CD269 (BCMA), CD273, CD274, CD276, CD279, CD319, CD366 and CD371, interleukin receptors, especially preferred IL-8Rα (CXCR1), IL-8Rβ (CXCR2), IL-11Rα, IL-11Rβ, IL-13Rα1 and 2, CXCR4, c-Met, mesothelin, members of the epidermal growth factor receptor family, especially preferred ErbB1, ErbB2, ErbB3 or ErbB4; members of the tumor necrosis factor receptor superfamily, ephrins, ephrin receptors, especially preferred EphA1-10, EphA5 or EphB1-6, prostate specific antigens prostate stem cell antigen (PSCA) and prostate specific membrane antigen (PSMA), embryonic antigens carcinoembryonic antigen (CEA) and fetal acethylcholine receptor, members of the vascular endothelia growth factor family, epithelia cell adhesion molecule EpCAM, alphafetoprotein AFP, members of the intercellular adhesion molecule family, members of the mucin protein family, follicle stimulating hormone receptor (FSHR), the human high molecular weight-melanoma-associated antigen (HMW-MAA), folate binding protein FBP, folate receptors, somatostatin receptors, ligands of the NKG2D receptor, cytokine receptors, members of the epithelia glycoprotein family, diasialogangliosides, glypicans, G protein-coupled receptors, members of the carbonic anhydrase family, members of the carbohydrate antigen family, Notch ligands, melanoma-associated chondroitin sulfate proteoglycan (MCSP), glycoprotein A33, guanylate cyclase 2C and tumor-specific glycans, and
b) administering to the mammal an effective amount of a vector or a cell comprising a nucleotide sequence encoding a switchable chimeric antigen receptor,
wherein the switchable chimeric antigen receptor comprises three domains, wherein
   the first domain is a tag-binding domain or tag,
   the second domain is an extracellular hinge and a transmembrane domain and
   the third domain is a signal transduction domain,
wherein the tag-binding domain of the targeting module binds to the tag of the switchable chimeric antigen receptor or the tag of the targeting module binds to the tag-binding domain of the switchable chimeric antigen receptor, and
c) administering to a mammal an effective amount of a targeting module according to the invention,
wherein the targeting module according to the invention and the at least one further targeting module comprise different target cell binding domains, and identical tag-binding domains or tags, wherein the method is carried out in the order of the steps a), b) and c).

In preferred embodiments, the targeting module is administered on its own, preferably one hour to 2 days, more preferably 4 to 24 hours, prior to the administration of thevector or cell comprising a nucleotide sequence encoding a switchable chimeric antigen receptor and the targeting module according to the invention is administered, preferably in the range of 3 days to 30 days, after the administration of the vector or cell comprising a nucleotide sequence encoding a switchable chimeric antigen receptor. Furthermore, additional such doses of the targeting module may be administered following resting periods to reactivate the switchable chimeric antigen receptor carrying effector cells.

In further embodiments, the method for stimulating a chimeric antigen receptor mediated immune response in a mammal, preferably the method for treatment of cancer, infectious or autoimmune disease, comprises the following steps:
a) administering to a mammal an effective amount of a vector or a cell comprising an inducible expression system, preferably selected from Tet-On, Tet-On Advanced transactivator, Tet-On 3G or a promoter responsive to NFAT; and a nucleotide sequence encoding a switchable chimeric antigen receptor, wherein the switchable chimeric antigen receptor comprises three domains, wherein
   the first domain is a tag-binding domain or tag,
   the second domain is an extracellular hinge and a transmembrane domain and
   the third domain is a signal transduction domain, and
   a nucleotide sequence encoding a targeting module according to the invention, wherein the tag-binding domain of the targeting module binds to the tag of the switchable chimeric antigen receptor or the tag of the targeting module binds to the tag-binding domain of the switchable chimeric antigen receptor, and
b) administering to a mammal an effective amount of an inducer, preferably tetracycline or a derivative of tetracycline, e.g. doxycycline.

In embodiments, the inducible expression system induces the transcription of the nucleotide sequence encoding a switchable chimeric antigen receptor and/or a nucleotide sequence encoding a targeting module according to the invention, preferably the inducible expression system induces the transcription of the nucleotide sequence encoding a targeting module according to the invention.

In preferred embodiments, the method for stimulating a chimeric antigen receptor mediated immune response in a mammal, preferably the method for treatment of cancer, infectious or autoimmune disease, comprises the following steps:
a) administering to a mammal an effective amount of a targeting module comprising at least one target cell binding domain and a tag-binding domain or a tag, wherein the at least one target cell binding domain is an antibody, antibody fragment, a protein, a peptide or a low molecular weight organic ligand that binds to surface antigens selected from the group comprising CD2, CD3, CD4, CD8, CD10, CD19, CD20, CD22, CD23, CD25, CD30, CD33, CD38, CD44, CD44v6 CD52, CD90, CD99, CD123, CD133, CD150 CD181, CD182, CD184, CD223, CD229, CD269 (BCMA), CD273, CD274, CD276, CD279, CD319, CD366 and CD371, interleukin receptors, especially preferred IL-8Rα (CXCR1), IL-8Rβ (CXCR2), IL-11Rα, IL-11Rβ, IL-13Rα1 and 2, CXCR4, c-Met, mesothelin, members of the epidermal growth factor receptor family, especially preferred ErbB1, ErbB2, ErbB3 or ErbB4; members of the tumor necrosis factor receptor superfamily, ephrins, ephrin receptors, especially preferred EphA1-10, EphA5 or EphB1-6, prostate specific antigens prostate stem cell antigen (PSCA) and prostate specific membrane antigen (PSMA), embryonic antigens carcinoembryonic antigen (CEA) and fetal acethylcholine receptor, members of the vascular endothelia growth factor family, epithelia cell adhesion molecule EpCAM, alphafetoprotein AFP, members of the intercellular adhesion molecule family, members of the mucin protein family, follicle stimulating hormone receptor (FSHR), the human high molecular weight-melanoma-associated antigen (HMW-MAA), folate binding protein FBP, folate receptors, somatostatin receptors, ligands of the NKG2D receptor, cytokine receptors, members of the epithelia glycoprotein family, diasialogangliosides, glypicans, G protein-coupled receptors, members of the carbonic anhydrase family, members of the carbohydrate antigen family, Notch ligands, melanoma-associated chondroitin sulfate proteoglycan (MCSP), glycoprotein A33, guanylate cyclase 2C and tumor-specific glycans, and
b) administering to a mammal an effective amount of a vector or a cell comprising an inducible expression system, preferably selected from Tet-On, Tet-On Advanced transactivator, Tet-On 3G or a promoter responsive to NFAT; and a nucleotide sequence encoding a switchable chimeric antigen receptor, wherein the switchable chimeric antigen receptor comprises three domains, wherein
   the first domain is a tag-binding domain or tag,
   the second domain is an extracellular hinge and a transmembrane domain and
   the third domain is a signal transduction domain, and
   a nucleotide sequence encoding a targeting module according to the invention, wherein the tag-binding domain of the targeting module binds to the tag of the switchable chimeric antigen receptor or the tag of the targeting module binds to the tag-binding domain of the switchable chimeric antigen receptor and,
c) administering to a mammal an effective amount of an inducer, preferably tetracycline or a derivative of tetracycline, e.g. doxycycline,
wherein the targeting module according to the invention and the at least one further targeting module comprise different target cell binding domains, and identical tag-binding domains or tags, wherein the method is carried out preferably in the order of the steps a), b) and c).

In further embodiments, the preceding embodiments can be combined.

The present invention will now be further explained by the following non-limiting figures and examples.

FIG. 1 shows a schema of a switchable chimeric antigen receptor (CAR) with three domains, wherein the first domain is a tag-binding domain or a tag (exemplified as scFv), the second domain is an extracellular hinge and a transmembrane domain and the third domain is a signal transduction domain, and the optional fourth domain is a short peptide linker in the extracellular portion of the receptor.

FIG. 2 shows results of flow-cytometry based in vitro binding assays of three different PD-L1 and/or PD-L2 binding TMs to PD-L1 expressing cells. Data are presented as mean ± SD of at least four independent experiments.

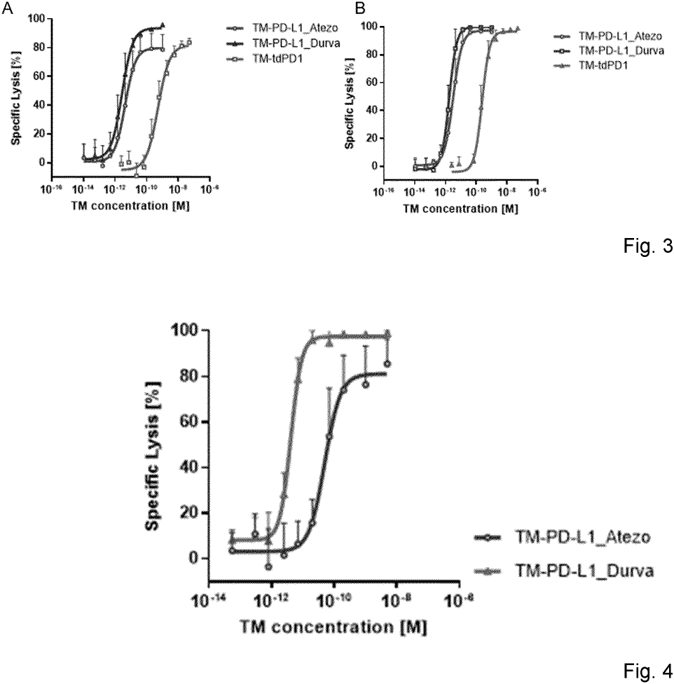

FIG. 3 shows results of flow-cytometry based in vitro killing assays of switchable chimeric antigen receptor T cells redirected by three different PD-L1 and/or PD-L2 binding TMs to PD-L1 expressing cells. Target cell numbers were determined after incubation for A) 24 h and B) 48 h. Data are presented as mean ± SD of four independent experiments with individual T cell donors.

FIG. 4 shows results of live-cell microscopy in vitro killing assays of switchable chimeric antigen receptor T cells redirected by two different anti-PD-L1 TMs to interferon-y treated PC3-PSCA cells. Data are presented as mean ± SD of four independent experiments with individual T cell donors.

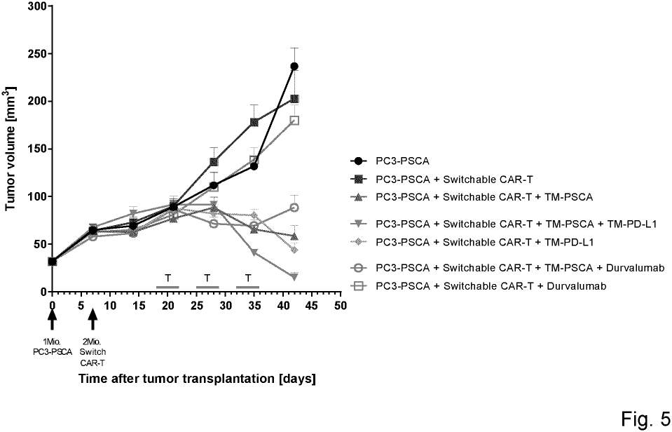

FIG. 5 shows results of in vivo treatment of immunodeficient mice subcutaneously transplanted with PC3-PSCA tumors and treated with switchable chimeric antigen receptor T cells alone or in different combinations with an anti-PSCA TM, an anti-PD-L1 TM based on Durvalumab or Durvalumab. Data are presented as mean ± SEM of eleven mice per group.

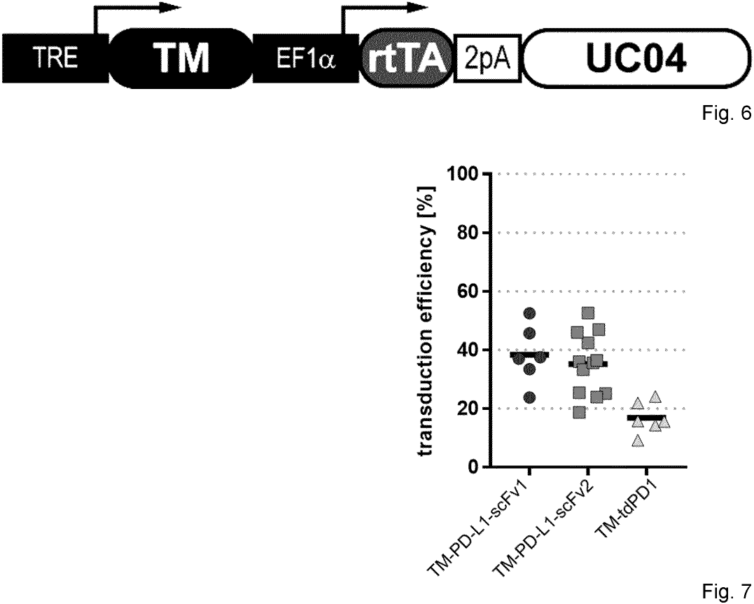

FIG. 6 shows a schema of combined UniCAR and TetOn-inducible immune checkpoint specific targeting module expression cassette (TRE-tetracycline response element, EF1a-elongation factor 1a, rtTA-reverse tetracycline transactivator, iRFP-infrared fluorescent protein, eGFP-enhanced green fluorescent protein).

FIG. 7 shows the frequency of gene modified primary human T cells obtained by lentiviral gene transfer: doxycycline- inducible gene expression of PD-L1 binding targeting modules under the control of a TetOn-cassette combined with a UniCAR. Data shown represent at least six batches obtained from individual human T cell donors.

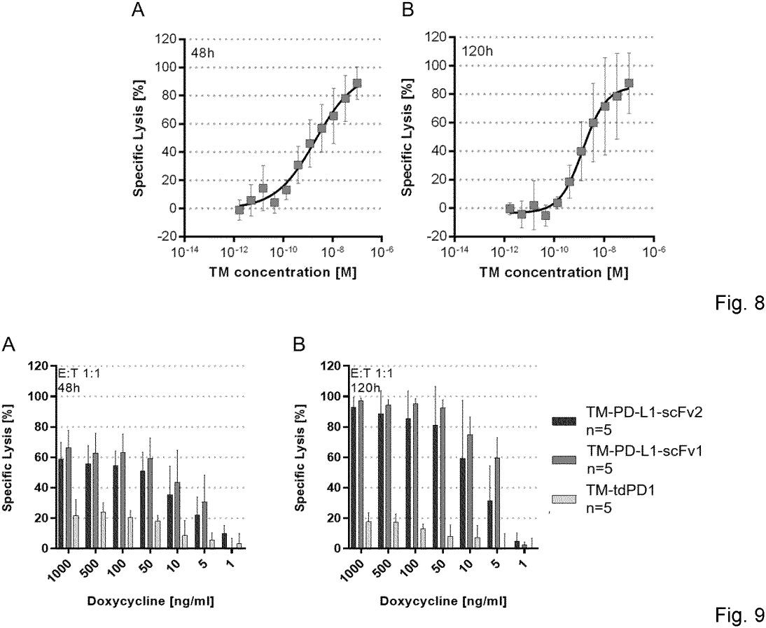

FIG. 8 shows targeting module-dependent cytotoxic response of human T cells genetically engineered to express UniCAR and a PD-L1 and/or PD-L2 binding TM under control of a doxycycline-responsive TetOn-cassette A) after 48 h and B) after 120 h. Mean ± SD of results obtained with six independent human donors are shown.

FIG. 9 shows flow cytometry-based cytotoxicity assay to assess lysis of PD-L1 positive Nalm-6 cells mediated by UniCAR-T modified to secrete PD-L1 and/or PD-L2 binding TM A) after 48 h and B) after 120 h. Data are presented as mean ± SD of at least four independent experiments with individual T cell donors, respectively.

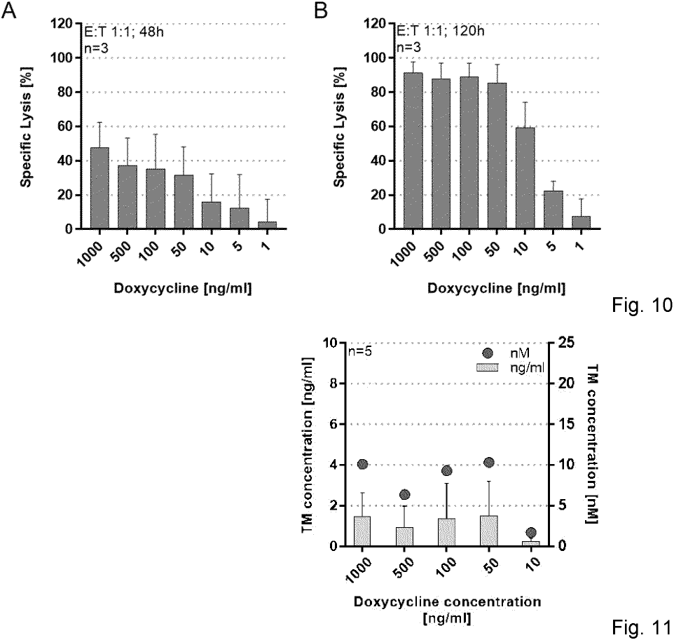

FIG. 10 shows a cytotoxicity assay of human T cells expressing UniCAR and a PD-L1 and/or PD-L2 binding TM under control of doxycycline-responsive TetOn-cassette against intrinsically PD-L1 up-regulating PC3-PSCA cells A) after 48 h and B) after 120 h. Data are shown as mean ± SD of three repetitive experiments with individual T cell donors, respectively.

FIG. 11 shows the quantification of TM-PD-L1-scFv1 concentrations secreted by gene-engineered human T cells upon doxycycline induction. Data are presented as mean ± SD of five repetitive experiments with five individual T cell donors.

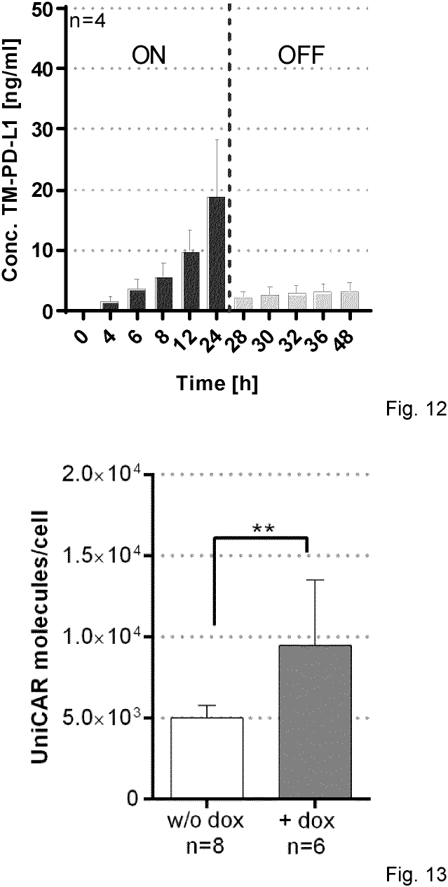

FIG. 12 shows kinetics of On/Off switch of doxycycline-inducible TM-PD-L1-scFv1 secretion. Data are presented as mean ± SD of four individual T cell donors and experiments.

FIG. 13 shows the quantification of UniCAR molecules on the surface in dependence on activation by doxycycline-induced expression of TM-PD-L1-scFv1. Mean ± SD of three individual T cell donors are shown, statistical significance was calculated by paired t-test, ** p < 0.01.

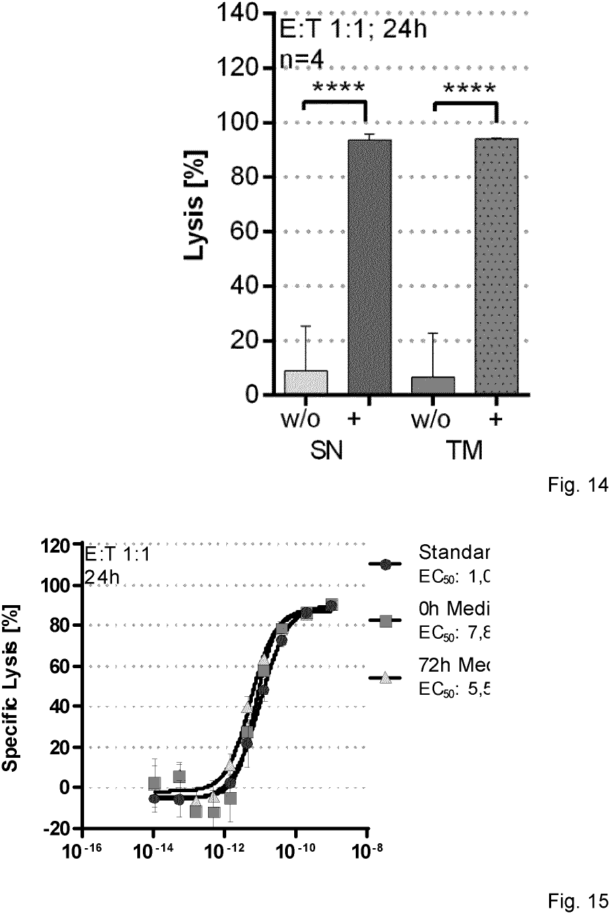

FIG. 14 shows that basal expression of TM-PD-L1-scFv under control of Tet On-cassette is below threshold for cytotoxicity induction. Human T cells genetically modified to express a switchable chimeric antigen receptor and to secret TM-PD-L1-scFv in response to doxycycline were cultured in standard culture medium in absence (SN w/o) or presence of 1000 ng/ml doxycycline (SN +) for 72 hours. Supernatant was harvested, diluted 1:5, and added to Nalm-6-PD-L1 target cells and human T cells genetically modified to express a switchable chimeric antigen receptor alone. As additional controls, cell mix was incubated with 1 nM purified TM-PD-L1-scFv1 (+TM) or plain cell culture medium (w/o TM). After incubation for 24 h, lysis of target cells was analyzed using flow cytometry. Data presented as mean ± SD of four individual T cell donors. Statistical significance was calculated by One-way ANOVA; **** p<0.0001.

FIG. 15 shows stability of TM-PD-L1-scFv1 in culture medium at 37° C. for 72 h followed by flow cytometry-based cytotoxicity assay.

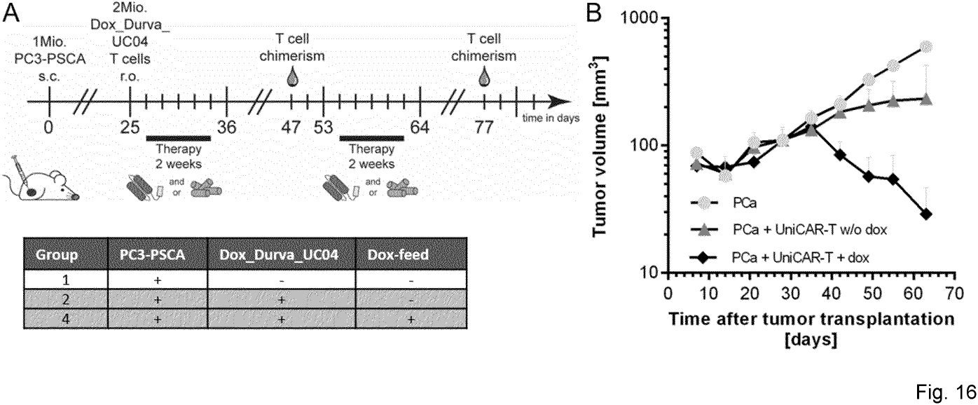

FIG. 16 shows the in vivo efficacy of human T cells genetically engineered to express UniCAR and TM-PD-L1-scFv under control of TetOn-cassette in a xenotransplantation model of prostate cancer (PCa). A) Schematic illustration of the experimental set-up. B) Measured tumor volume plotted as a function of time as mean ± SEM of at least nine mice.

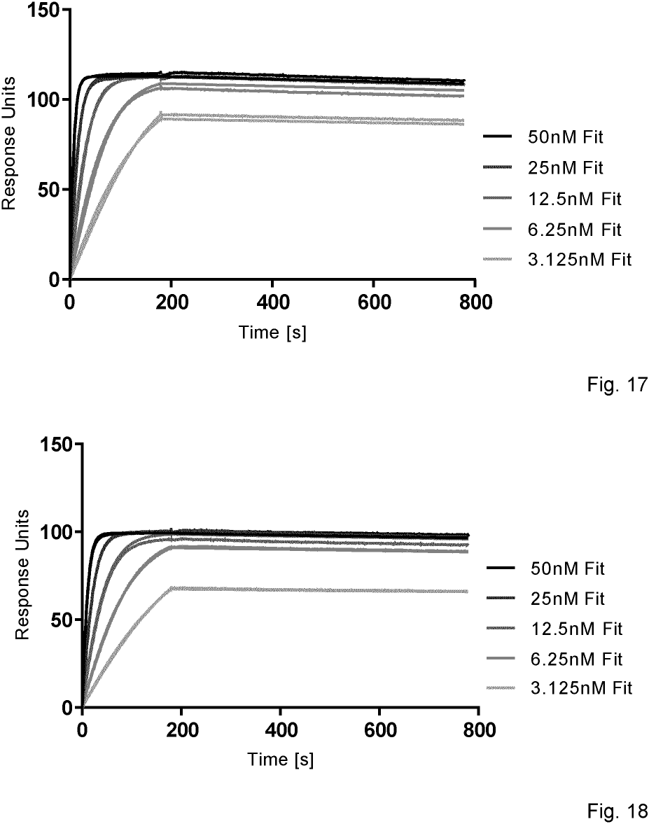

FIG. 17 shows the surface plasmon resonance (SPR) measurements of the binding of TM-PD-L1-scFv1 to recombinant human Fc-tagged PD-L1.

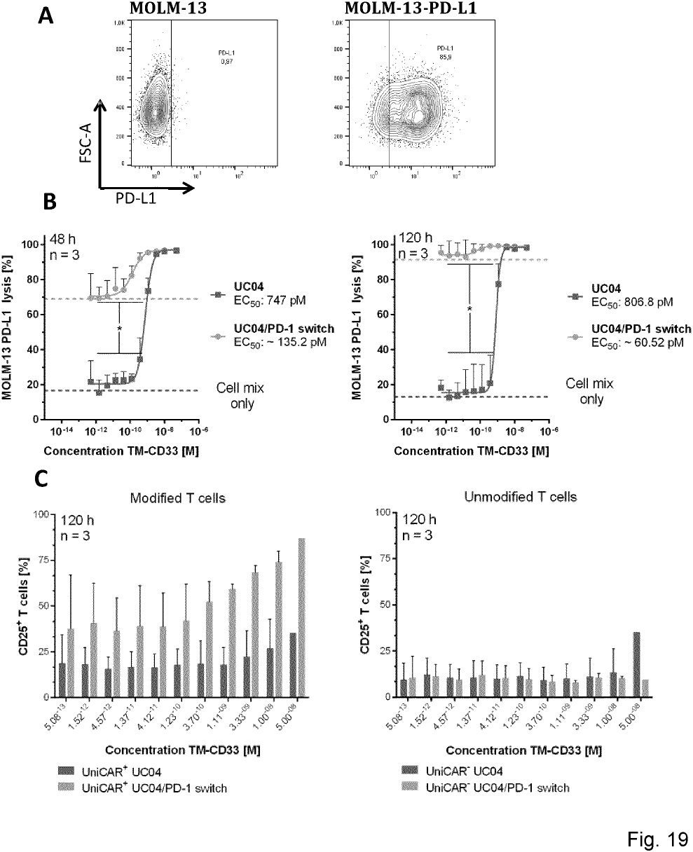

FIG. 18 shows the surface plasmon resonance (SPR) measurements of the binding of TM-PD-L1-scFv2 to recombinant human Fc-tagged PD-L1.

FIG. 19 shows the in vitro elimination of the acute myeloid leukemia cell line MOLM-13 expressing recombinant PD-L1 by UniCAR and UniCAR/PD-1 switch-modified T cells. A) MOLM-13 cells were engineered to express recombinant PD-L1 and analyzed for PD-L1 surface expression via flow cytometry. B) UniCAR-T cells (CD137/CD3-ζ, UC04) or UniCAR-PD-1 switch T cells were co-cultured with MOLM-13-PD-L1 at an effector to target cell (E:T) ratio of 2:1 in absence or presence of a CD33-specific targeting module (TM CD33). Cytotoxic reactivity against MOLM-13-PD-L1 was assessed after 48 h and 120 h via flow cytometry (mean ± SD). C) Analysis of T cell activation by surface staining of the T cell activation marker CD25 after 120 h of co-cultivation with MOLM-13-PD-L1 (mean ± SD). Statistical significance for n = 3 independent T cell donors was assessed by the Wilcoxon-signed rank-test (* P<0.05, ns = not significant).

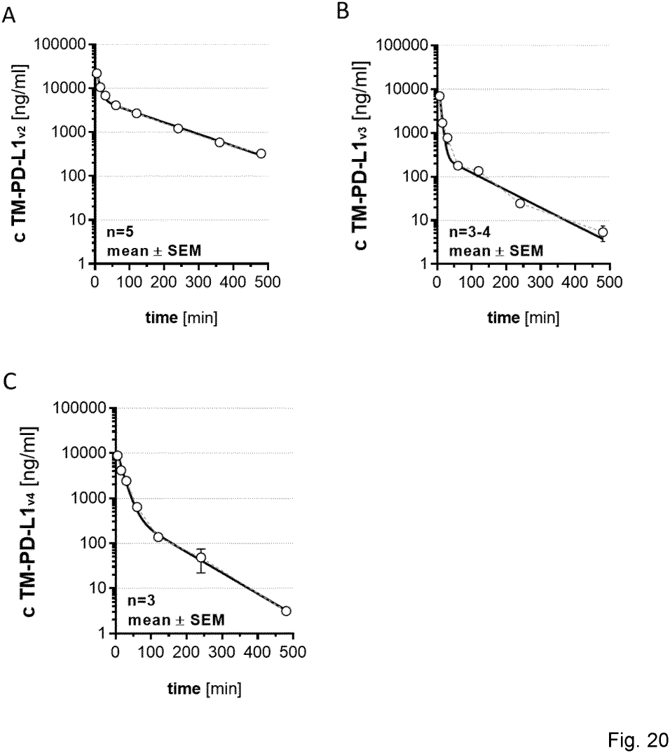

FIG. 20 shows pharmacokinetic studies of TM-PDL1 variants in NSG mice by determining plasma concentrations of TM-PD-L1 variants via ELISA: A) TM-PD-L1-scFv2, B) TM-PD-L1-scFv3 and C) TM-PD-L1-scFv4. Data is shown for three to four mice per time point analyzed.

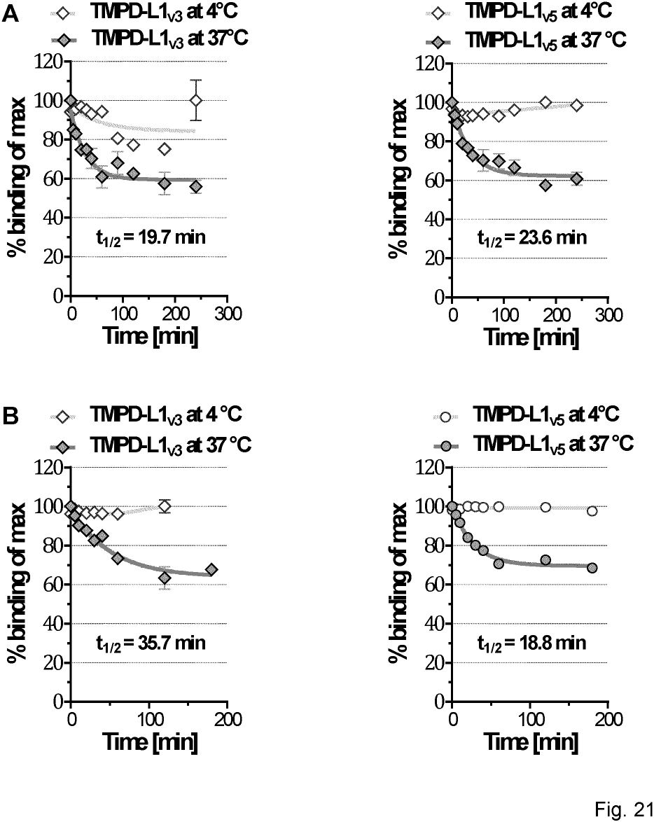

FIG. 21 shows target-mediated internalization of PD-L1-specific TMs on PD-L1-expressing cell lines: In order to determine the PD-L1-mediated pharmacodynamic half-life of PD-L1-specific soluble adapters, the PD-L1-positive target cell lines (A) DU-145 and (B) U-251 were stained with 1000 nM TM-PD-L1-scFv3 (TMPD-L1ᵥ₃) or TM-PD-L1-scFv5 (TMPD-L1ᵥ₅) and incubated for indicated time points at either 4° C. or 37° C. Data is shown as % binding of max by normalizing the obtained MFI to the highest MFI measured. Additionally, the pharmacodynamic half-life (t_{½}) was calculated for each TM at 37° C. Data is shown as mean ± SD of one experiment measured in technical triplicates.

### PRODUCTION OF THE SWITCHABLE CAR CELL

The immune cells can be genetically engineered to express switchable CARs and/or inducible TMs directed against PD-L1 and/or PD-L2 by various methods. A polynucleotide vector encoding the switchable CAR and all necessary elements to ensure its expression in the genetically engineered immune cell is transferred into the immune cell. The transfer of the vector can be performed by electroporation or transfection of nucleic acids or the help of viral vector systems like adeno-, adeno-associated, retro-, foamy- or lentiviral viral gene transfer.

The lentiviral gene transfer is applied for stable expression of switchable CARs in immune cells by first constructing a lentiviral vector encoding for a selected switchable CAR. The lentiviral vector is pLVX-EF1alpha UniCAR 28/ζ (Clontech, Takara Bio Group), in which the lentiviral parts of the vector are derived from the human immunodeficiency virus (HIV) and the MSC/IRES/ZxGreenl portion was replaced by the switchable CAR construct.

The lentiviral particles are produced by transient transfection of human embryonal kidney (HEK) 293T (ACC 635) cells with the switchable CAR encoding lentiviral vector plasmid and cotransfection with a group specific antigen (gag) and Polymerase (pol) encoding plasmid (psPAX2) plus a plasmid encoding for an envelope (pMD2.G). After transfection, the packaging plasmid expresses Gag and Pol protein of HIV-1. The plasmid MD2.G encodes the glycoprotein of the vesicular stomatitis virus (VSV-G). VSV-G protein is used to lentiviral vectors to transduce a broad range of mammalian cells. Various envelopes from different virus species can be utilized for this purpose. Lentiviral vectors can successfully pseudotype with the envelope glycoproteins (Env) of amphotropic murine leukemia virus (MLV) or the G protein of vesicular stomatitis virus (VSV-G), a modified envelope of the prototypic foamy virus (PFV) or chimeric envelope glycoprotein variants derived from gibbon ape leukemia virus (GaLV) and MLV.

Supernatants from transfected HEK293T cells are harvested 24 h to 96 h after transfection and virus particles are concentrated from the supernatant by ultracentrifugation or other methods. For lentiviral transduction of immune cells, peripheral blood mononuclear cells (PBMC) or isolated T cells are activated with mab specific for the CD3 complex, e.g. clone OKT3 or UCHT1, either given in solution or coated to plastic cell culture dishes or magnetic beads or a biodegradable polymer matrix. Activation of PBMC or isolated T cells is further enhanced by stimulating costimulatory pathways with mabs or ligands specific for CD27, CD28, CD134 or CD137 either alone or in combinations coated to plastic cell culture dishes or magnetic beads or a biodegradable polymer matrix and the supply with exogenous recombinant cytokines like interleukin (IL)-2, IL-7, IL-12, IL-15 and IL-21. Concentrated or non-concentrated virus particles are added to PBMC or T cell cultures 24 h to 96 h after initial administration of activating CD3 specific antibodies and/or antibodies specific for costimulatory receptors CD27, CD28, CD134 or CD137 and/or recombinant cytokines as single or multiple doses. T cell electroporation, transduction and expansion may be performed in open cell culture systems by manual handling or in closed partially or fully automated systems.

Stable transduction of T cells may be determined by flow cytometry after staining with tag-containing molecules for surface expression of switchable CARs or mabs directed against the fourth domain of switchable CARs from day 3 onwards after final administration of virus supernatant. Switchable CAR transduced T cells can be propagated in vitro by culturing them under supply of recombinant cytokines and activating anti-CD3 mabs.

In case the switchable CAR harbors the optional fourth domain, a peptide sequence forming a linear epitope for a mab, immune cells genetically modified to express switchable CARs can be specifically propagated in vitro by coating a mab or antibody fragments thereof binding to the fourth switchable CAR domain to the surface of culture dishes or to beads of any kind or a biodegradable polymer matrix, which are added to the cell culture at a defined ratio. The binding of surface-coated mabs to the switchable CAR peptide domain induces cross-linkage of cell-surface expressed switchable CARs and formation of an immune synapse, which leads to the activation of signal pathways specifically triggered by the signal domain of the switchable CAR. Depending on the signal pathways induced, this may lead to enhance proliferation and sustained resistance against activation-induced cell death of the switchable CAR carrying immune cells and therefore enrichment of switchable CAR genetically modified immune cells in a mixed population.

The optional fourth domain, a peptide sequence forming a linear epitope for a mab, can be further utilized to enrich and purify switchable CAR expressing immune cells from mixed populations. Enrichment and purification is performed with the help of a mab or antibody fragment thereof binding to the fourth switchable CAR domain to either mark switchable CAR expressing cells for cell sorting or to transiently link the switchable CAR expressing immune cell to small particles, which can be utilized for cell isolation. In one aspect, switchable CAR engrafted immune cells are incubated with the mab recognizing the fourth domain. Next, magnetic beads are added, which are conjugated with antibodies or fragments thereof directed against the species- and isotype specific heavy and light chains of the mab binding to the optional fourth domain. Thus, switchable CAR expressing immune cells and magnetic beads are linked and are trapped and separated from other immune cells in a magnetic field.

### Design of Targeting Modules According to the Invention

In one example, the PD-L1 and/or PD-L2 binding TM comprises an scFv of Atezolizumab according to SEQ ID No. 57, comprising SEQ IDs No. 6 and 7 connected by a linker and the human La epitope E5B9 according to SEQ ID No. 14 (TM-PD-L1-scFv1).

In an alternative example, the PD-L1 and/or PD-L2 binding TM comprises an scFv of Atezolizumab according to SEQ ID No. 65, comprising SEQ IDs No. 6 and 7 connected by a linker and the human La epitope E5B9 according to SEQ ID No. 14.

In further examples, the PD-L1 and/or PD-L2 binding TM comprises an scFv of Durvalumab according to SEQ ID No. 58, comprising SEQ IDs No. 8 and 9 connected by a linker and the human La epitope E5B9 according to SEQ ID No. 14 (TM-PD-L1-scFv2).

In an alternative example, the PD-L1 and/or PD-L2 binding TM comprises an scFv of Durvalumab according to SEQ ID No. 66, comprising SEQ IDs No. 8 and 9 connected by a linker and the human La epitope E5B9 according to SEQ ID No. 14.

In a further example, the PD-L1 and/or PD-L2 binding TM comprises an scFv of BMS-936559 according to SEQ ID No. 86, comprising SEQ IDs No. 4 and 5 connected by a linker and the human La epitope E5B9 according to SEQ ID No. 14 (TM-PD-L1-scFv3).

In an alternative example, the PD-L1 and/or PD-L2 binding TM comprises an scFv of BMS-936559 according to SEQ ID No. 85, comprising SEQ IDs No. 4 and 5 connected by a linker and the human La epitope E5B9 according to SEQ ID No. 14.

In a further example, the PD-L1 and/or PD-L2 binding TM comprises an scFv of BMS-936559 according to SEQ ID No. 97, comprising SEQ IDs No. 77 and 78 connected by a linker and the human La epitope E5B9 according to SEQ ID No. 14 (TM-PD-L1-scFv4).

In an alternative example, the PD-L1 and/or PD-L2 binding TM comprises an scFv of BMS-936559 according to SEQ ID No. 98, comprising SEQ IDs No. 77 and 78 connected by a linker and the human La epitope E5B9 according to SEQ ID No. 14.

In a further example, the PD-L1 and/or PD-L2 binding TM comprises an scFv of BMS-936559 according to SEQ ID No. 87, comprising SEQ IDs No. 67 and 68 connected by a linker and the human La epitope E5B9 according to SEQ ID No. 14 (TM-PD-L1-scFv5).

In an alternative example, the PD-L1 and/or PD-L2 binding TM comprises an scFv of BMS-936559 according to SEQ ID No. 88, comprising SEQ IDs No. 67 and 68 connected by a linker and the human La epitope E5B9 according to SEQ ID No. 14.

In further examples, the PD-L1 and/or PD-L2 binding TM is bivalent and comprises two PD-1 extracellular domains, each of which is a fragment of SEQ ID No. 3 and the human La epitope E5B9 according to SEQ ID No. 14 (TM-tdPD1). TM-tdPD-1 is encoded by SEQ ID No. 59.

### Characterization of the Functionality of the Targeting Modules According to the Invention

The functionality of the PD-L1 and/or PD-L2 binding TMs can be confirmed in binding assays to human and murine PD-L1 using surface plasmon resonance as well as in cell-based cytotoxicity assays.

The results of flow-cytometry based in vitro binding assays of three different PD-L1 and/or PD-L2 binding TMs to Nalm-6 cells transduced to express PD-L1 are shown in FIG. 2. Data are presented as mean ± SD of at least four independent experiments.

The results of flow-cytometry based in vitro killing assays of switchable chimeric antigen receptor T cells (SEQ ID No. 60) redirected by three different PD-L1 and/or PD-L2 binding TMs to Nalm-6 cells transduced to express PD-L1 are shown in FIG. 3. Effector to target ratio (E:T) was 1:1. Target cell numbers were determined after incubation for A) 24 h and B) 48 h. Data are presented as mean ± SD of four independent experiments with individual T cell donors.

The results of live-cell microscopy based in vitro killing assays of switchable chimeric antigen receptor T cells (SEQ ID No. 60) redirected by two different anti-PD-L1 TMs to interferon-y treated PC3-PSCA cells are shown in FIG. 4. Effector to target ratio (E:T) was 1:1. Target cell numbers were determined after incubation for 96 h. Data are presented as mean ± SD of four independent experiments with individual T cell donors.

The results of in vivo treatment of immunodeficient mice subcutaneously transplanted with PC3-PSCA tumors and treated with switchable chimeric antigen receptorT cells (SEQ ID No. 60) alone or in different combinations with an anti-PSCA TM, an anti-PD-L1 TM based on Durvalumab or Durvalumab are shown in FIG. 5. The switchable chimeric antigen receptor T cells were injected intravenously at the indicated time point. The TMs were injected twice daily intraperitoneally and Durvalumab was injected once intraperitoneally during each of the indicated treatment periods (T). Data are presented as mean ± SEM of eleven mice per group.

### Design of a Combined Switchable CAR and Inducible, Targeting Module Expression Cassette (FIG. 6)

The lentiviral gene expression plasmid contains an EF1a promoter for continuous polycistronic transcription of rtTA and UniCAR, separated by a Thosea asigna virus derived peptide cleavage site (2pA). In addition, a PD-L1 and/or PD-L2 binding targeting module (TM) is under the control of a TRE3G promoter, which will only be activated by binding of rtTA in presence of doxycycline. This arrangement allows a doxycyclin-dependent inducible and reversible gene-expression.

### Characterization of the Functionality of the Combined Switchable CAR and Inducible, Targeting Module Expression Cassette

The frequency of gene modified primary human T cells obtained by lentiviral gene transfer is shown in FIG. 7. T cells isolated from voluntary healthy human donors were transduced with lentiviral supernatant transferring genetic information for doxycycline-inducible gene expression of PD-L1 and/or PD-L2 binding targeting modules (TM-PD-L1-scFv-1, TM-PD-L1-scFv2 and TM-tdPD1) under the control of a TetOn-cassette combined with UniCAR. Transduction efficiency at day 14 after transduction was analyzed via immunofluorescence staining of surface displayed UniCAR using a primary mouse a7B6-mAb (10 µg/ml) directed against the extracellular E7B6-tag integrated into the UniCAR hinge. An AlexaFluor 647 conjugated goat-a-mouse Ab was used as secondary Ab.

The TM-dependent cytotoxic response of human T cells genetically engineered to express UniCAR and the TM according to the invention under control of a doxycycline-responsive TetOn-cassette is shown in FIG. 8. T cells isolated from voluntary healthy human donors were genetically modified to express UniCAR and a PD-L1 and/or PD-L2 binding targeting module, in particular TM-PD-L1-scFv2, under the control of a TetOn-cassette. The ability of the UniCAR modified cells to mediate TM-dependent cytotoxic response against target cancer cells was evaluated in a flow-cytometry based cytotoxicity assay. Nalm-6-PSCA cells were cultured with human UniCAR expressing T cells at a total T cell to target cell ratio of 1:1 in the presence of increasing concentrations of a PSCA binding TM comprising a PSCA binding domain according to EP 2 990 416 B1; and the human La epitope E5B9 according to SEQ ID No. 14. At indicated time points the number of living target cells was determined. Specific lysis was calculated relative to a control without addition of TM.

The results of the cytotoxicity assay to assess lysis of PD-L1 positive Nalm-6 cells mediated by UniCAR-T modified to secrete PD-L1 specific TM are shown in FIG. 9. Nalm-6-PD-L1 target cells were cultured with genetically modified human T cells in a total T cell to target cell (E:T) ratio of 1:1 in presence of decreasing concentrations of doxycycline ranging from 1,000 to 1 ng/ml. T cells were engineered to express UniCAR and a doxycycline-responsive TetOn-cassette to induce expression and secretion of TM-PD-L1-scFv-1, TM-PD-L1-scFv2 or TM-tdPD1 in presence of doxycycline. Specific lysis of target cells was analyzed after A) 48 h and B) 120 h of incubation and normalized to background lysis without doxycycline.

FIG. 10 shows the results of the cytotoxicity assay of human T cells expressing UniCAR and a PD-L1 specific TM under control of doxycycline-responsive TetOn-cassette against intrinsically PD-L1 up-regulating PC3-PSCA cells. PC3-PSCA cells were treated with 25 ng/ml IFN-y to induce PD-L1 up-regulation. Subsequently, PD-L1 presenting PC3-PSCA were incubated with genetically modified T cells expressing UniCAR and a PD-L1-specific TM, in particular TM-PD-L1-scFv1, under the control of doxycycline inducible TetOn-cassette in a total T cell to target cell (E:T) ratio of 1:1 for A) 48 h and B) 120 h. Doxycycline was added in decreasing concentrations from 1000 to 1 ng/ml and specific lysis was calculated relative to lysis of target cells without doxycycline.

The quantification of TM-PD-L1-sFv1 concentrations secreted by gene-engineered human T cells upon doxycycline induction (FIG. 11) was carried out by incubating PD-L1 positive target cells and human T cells engineered to express UniCAR and a TetOn-cassette for doxycycline inducible TM-PD-L1-scFv1 in a total T cell to target cell (E:T) ratio of 1:1 with decreasing concentration of doxycycline for 120 h. Cell-free supernatant was harvested and secreted TM-PD-L1-scFv1 concentrations were analyzed by ELISA. Doxycycline concentration is blotted against TM concentration in ng/ml and resulting molar concentration, respectively.

The kinetics of On/Off switch of doxycycline-inducible TM-PD-L1-scFv1 secretion is shown in FIG. 12. For each indicated time point of the ON-phase 1×10⁵ human T cells genetically engineered to express UniCAR and TM-PD-L1-scFv1 under the control of doxycycline (dox) inducible TetOn-cassette were incubated in 500 µl culture medium in the presence of 1000 ng/ml doxycycline. Likewise, for the OFF-phase a pool of the engineered T cells were first cultured with the same amount of doxycycline for 24 h, washed thoroughly afterwards and cultured as described for the ON-phase, but without addition of doxycycline for additional 4, 6, 8,12 and 24 h. For each time point analyzed, cell-free supernatant was harvested and TM-PD-L1-scFv1 levels were quantified using ELISA.

The quantification of UniCAR molecules on the surface in dependence on the activation by doxycycline-induced expression of TM-PD-L1-scFv1 is shown in FIG. 13. The number of UniCAR surface molecules on engineered T cells was quantified using the calibration curve generated with the DAKO QIFlKIT®. Immunofluorescence staining of gene-engineered T cells was performed using anti7B6-mAb for UniCAR detection and AlexaFluor 647 goat-a-mouse Ab as secondary Ab was applied to pre-stained T cells but calibration beads as well. Genetically engineered human T cells expressing UniCAR and TetOn-cassette for doxycycline-inducible TM-PD-L1-scFv1 were cultured in absence or presence of 1,000 ng/ml doxycycline for 24 hours.

FIG. 14 shows that the basal expression of TM-PD-L1-scFv under control of TetOn-cassette is below threshold for cytotoxicity induction. Human T cells genetically modified to express UniCAR and to secret TM-PD-L1-scFv in response to doxycycline were cultured in standard culture medium in absence (SN w/o) or presence of 1,000 ng/ml doxycycline (SN +) for 72 hours. Supernatant was harvested, diluted 1:5, and added to Nalm-6-PD-L1 target cells and human T cells genetically modified to express UniCAR alone. As additional controls, cell mix was incubated with 1 nM purified TM-PD-L1-scFv1 (+TM) or plain cell culture medium (w/o TM) instead. After incubation for 24 h lysis of target cells was analyzed using flow cytometry.

In order to verify stability of TM-PD-L1-scFv1 at 37° C., purified TM was incubated in culture medium at 37° C. for 72 h and potency was analyzed in a standard flow cytometry-based cytotoxicity assay using human T cells genetically modified to express UniCAR alone and Nalm-6-PD-L1 target cell in a total T cell to target cell (E:T) ratio of 1:1 for 24 h (FIG. 15).

The in vivo efficacy of human T cells genetically engineered to express UniCAR and TM-PD-L1-scFv under control of TetOn-cassette in a xenotransplantation model of prostate cancer (PCa) is shown in FIG. 16. The mice were transplanted subcutaneously with 1×10⁶ tumor cells (PC3-PSCA) (FIG. 16 A). Twenty-five days later 2 million T cells (of which 28.7% were genetically engineered to express UniCAR and TM-PD-L1-scFv under control of TetOn-cassette) were administered and doxycycline-feed was started the same day. Feeding of doxycycline was continued for two weeks in a row with a break of two days in between. Second treatment cycle was started two weeks later and performed in the same way. The tumor volume was measured once a week with a digital caliper and is blotted as a function of time as mean ± SEM of at least 9 mice (FIG. 16 B).

### Combination of a Switchable CAR Redirected With a Targeting Module Against CD33 With a PD-L1:CD28 Switch Receptor

The in vitro elimination of the acute myeloid leukemia cell line MOLM-13 expressing recombinant PD-L1 by UniCAR and UniCAR/PD-1 switch-modified T cells was examined by MOLM-13 cells engineered to express recombinant PD-L1 and analyzed for PD-L1 surface expression via flow cytometry (see FIG. 19 A).

UniCAR-T cells (CD137/CD3-ζ, UC04) or UniCAR-PD-1 switch T cells were co-cultured with MOLM-13-PD-L1 at an effector to target cell (E:T) ratio of 2:1 in absence or presence of a CD33-specific targeting module (TM CD33). Cytotoxic reactivity against MOLM-13-PD-L1 was assessed after 48 h and 120 h via flow cytometry (see FIG. 19 B, mean ± SD).

The T cell activation was analyzed by surface staining of the T cell activation marker CD25 after 120 h of co-cultivation with MOLM-13-PD-L1 (see FIG. 19 C, mean ± SD). Statistical significance for n = 3 independent T cell donors was assessed by the Wilcoxon-signed rank-test (* P<0.05, ns = not significant).

The PD-L1:CD28 switch receptor in combination with UniCAR (4-1BB-CD3zeta UniCAR) exhibits a high targeting module-independent lysis of MOLM-13 PD-L1 cells, pointing to limitations of such an approach.

### Pharmacokinetic Studies of TM-PDL1 Variants

For the pharmacokinetic studies of TM-PDL1 variants in NSG mice, TM-PD-L1 was administered via intravenous tail vein injection of 1 µg/g_{BW} of NSG mice. Peripheral blood was taken via retroorbital puncture at indicated time points. Plasma concentrations of TM-PD-L1 variants were determined via ELISA utilizing PDL1-Fc chimera capture protein and anti-penta-His HRPconjugate detection antibody (lower limit of quantification 0.03-0.22 ng/ml assay concentration). In FIG. 20 A), B), C) data is shown for three to four mice per time point analyzed. Indicated pharmacokinetic parameters were defined utilizing PK solver2.0 (Zhang et al. 2010) applying weighted (1/Y²) two-compartmental analysis. Slope of the terminal phase was calculated from the last three or four time points (t_{½}β). In FIG. 20 the concentration of A) TM-PD-L1-scFv2, B) TM-PD-L1-scFv3 and C) TM-PD-L1-scFv4 is shown and Tab. 1 shows the calculated pharmacokinetic (PK) parameters.

TABLE 1

Results from pharmacokinetic (PK) studies of TM-PDL1 variants: TM-PD-L1-scFv2 (TM-PD-L1ᵥ₂), TM-PD-L1-scFv3 (TM-PD-L1ᵥ₃) and TM-PD-L1-scFv4 (TM-PD-L1ᵥ₄).

| PK parameter | TM-PD-L1_{V2} | TM-PD-L1_{V3} | TM-PD-L1_{V4} |
|---|---|---|---|
| t_{½}α [min] | 6.18 | 3.9 | 10.58 |
| t_{½}β [min] | 112.08 | 75.4 | 65.26 |
| AUC_{0-∞} [µg/ml^{∗}h] | 1166.23 | 169.34 | 248.04 |

In FIG. 21 target-mediated internalization of PD-L1-specific TMs on PD-L1-expressing cell lines is shown. In order to determine the PD-L1-mediated pharmacodynamic half-life of PD-L1-specific soluble adapters, the PD-L1-positive target cell lines (A) DU-145 and (B) U-251 were stained with 1000 nM TM-PD-L1-scFv3 (TMPD-L1ᵥ₃) orTM-PD-L1-scFv5 (TMPD-L1ᵥ₅). Subsequently, stained target cells were incubated for indicated time points at either 4° C. or 37° C. Binding of PD-L1-specific TMs was analyzed via flow cytometry using an PE-labeled α-His Ab. Additionally, the pharmacodynamic half-life (t1/2) was calculated for each TM at 37° C.

### CITED NON-PATENT LITERATURE

Brudno JN, Kochenderfer JN (2016) Toxicities of chimeric antigen receptor T cells: recognition and management. Blood 127, 3321-3330.

Carpenito C, Milone MC, Hassan R, Simonet JC, Lakhal M, Suhoski, MM, Varela-Rohena A, Haines KM, Heitjan D, Albelda SM, Carrol RG, Riley JL, Pastan, I, June CH (2009) Control of large, established tumor xenografts with genetically retargeted human T cells containing CD28 and CD137 domains. Proc. Natl. Acad. Sci. USA. 106 (9), 3360-3365.

Cartellieri M, Bachmann M, Feldmann A, Bippes C, Stamova S, Wehner R, Temme A, Schmitz M (2010) Chimeric Antigen Receptor-Engineered T Cells for Immunotherapy of Cancer J. Biomed. Biotechnol. Article ID 956304, doi: 10.1155/2010/956304.

Cartellieri M, Feldmann A, Koristka S, Arndt C, Loff S, Ehninger A, von Bonin M, Bejestani EP, Ehninger G, Bachmann MP (2016) Switching CAR T cells on and off: a novel modular platform for retargeting of T cells to AML blasts. Blood cancer J. 6 (8), e458.

Cherkassky L, Morello A, Villena-Vargas J, Feng Y, Dimitrov DS, Jones DR, Sadelain M, Adusumilli PS (2017) Human CAR T cells with cell-intrinsic PD-1 checkpoint blockade resist tumor-mediated inhibition. J Clin Invest. 126 (8), 3130-3144.

Fedorov VD, Themeli M, Sadelain M (2013) PD-1- and CTLA-4-based inhibitory chimeric antigen receptors (iCARs) divert off-target immunotherapy responses. Sci. Transl. Med. 5 (215), 215ra172.

Finney HM, Akbar AN, Lawson AD (2004) Activation of resting human primary T cells with chimeric receptors: costimulation from CD28, inducible costimulator, CD134, and CD137 in series with signals from the TCR zeta chain. J. Immunol. 172 (1), 104-113.

Frigault MJ, Lee J, Basil MC, Carpenito C, Motohashi S, Scholler J, Kawalekar OU, Guedan S, McGettigan SE, Posey AD Jr, Ang S, Cooper LJ, Platt JM, Johnson FB, Paulos CM, Zhao Y, Kalos M, Milone MC, June CH (2015) Identification of chimeric antigen receptors that mediate constitutive or inducible proliferation of T cells. Cancer Immunol. Res. 3 (4), 356-367.

Gade TP, Hassen W, Santos E, Gunset G, Saudemont A, Gong MC, Brentjens R, Zhong XS, Stephan M, Stefanski J, Lyddane C, Osborne JR, Buchanan IM, Hall SJ, Heston WD, Rivière I, Larson SM, Koutcher JA, Sadelain M (2005) Targeted elimination of prostate cancer by genetically directed human T lymphocytes. Cancer Res. 65 (19), 9080-9088.

Gong MC, Latouche JB, Krause A, Heston WDW, Bander NH, Sadelain M (1999) Cancer patient T cells genetically targeted to prostate-specific membrane antigen specifically lyse prostate cancer cells and release cytokines in response to prostate-specific membrane antigen. Neoplasia. 1 (2), 123-127.

Grosser R, Cherkassky L, Chintala N, Adusumilli PS (2019) Combination Immunotherapy with CAR T Cells and Checkpoint Blockade for the Treatment of Solid Tumors. Cancer Cell. 36 (5), 471-482.

Grupp SA, Laos M, Barrett D, Aplenc R, Porter DL, Rheingold SR, Teachey DT, Chew A, Hauck B, Wright JF, Milone MC, Levine, BL, June CH (2013) Chimeric Antigen Receptor-Modified T Cells for Acute Lymphoid Leukemia. N. Engl. J. Med. 368, 1509-1518.

Guedan S, Posey AD, Shaw C, Wing A, Da T, Patel PR, McGettigan SE, Casado-Medrano V, Kawalekar OU, Uribe-Herranz M, Song D, Melenhorst JJ, Lacey SF, Scholler J, Keith B, Young RM, June CH (2018) Enhancing CAR T cell persistence through ICOS and 4-1BB costimulation. JCI Insight. 3(1), 96976.

Guedan S, Madar A, Casado-Medrano V, Shaw CE, Wing A, Liu F, Young RM, June CH, Posey AD (2020) Single residue in CD28-costimulated CAR T cells limits long-term persistence and antitumor durability. J Clin Invest. 133215.

Hombach AA, Abken H (2011) Costimulation by chimeric antigen receptors revisited the T cell antitumor response benefits from combined CD28-OX40 signalling. Int. J. Cancer. 129 (12), 2935-2944.

Hombach AA, Abken H (2013) Of chimeric antigen receptors and antibodies: OX40 and 41BB costimulation sharpen up T cell-based immunotherapy of cancer. Immunotherapy. 5 (7), 677-681. Hombach AA, Kofler D, Hombach A, Rappl G, Abken H (2007) Effective proliferation of human regulatory T cells requires a strong costimulatory CD28 signal that cannot be substituted by IL-2. J. Immunol. 179 (11), 7924-7931.

Hombach A, Sent D, Schneider C, Heuser C, Koch D, Pohl C, Seliger B, Abken H (2001) T-cell activation by recombinant receptors: CD28 costimulation is required for interleukin 2 secretion and receptor-mediated T-cell proliferation but does not affect receptor-mediated target cell lysis. Cancer Res. 61 (5), 1976-1982.

Kagoya Y, Tanaka S, Guo T, Anczurowski M, Wang CH, Saso K, Butler MO, Minden MD, Hirano N (2018) A novel chimeric antigen receptor containing a JAK-STAT signaling domain mediates superior antitumor effects. Nat Med. 24(3), 352-359.

Kershaw MH, Westwood JA, Parker LL, et al. (2006) A phase I study on adoptive immunotherapy using gene-modified T cells for ovarian cancer. Clin. Cancer Res. 12 (20), 6106-6115.

Lamers CH, Sleijfer S, Willemsen RA, Debets R, Kruit WHJ, Gratama JW, Stoter G (2004) Adoptive immuno-gene therapy of cancer with single chain antibody [scFv(lg)] gene modified T lymphocytes. J. Biol. Regul. Homeost. Agents. 18 (2), 134-140.

Lamers CH, Sleijfer S, Vulto AG, Kruit WH, Kliffen M, Debets R, Gratama JW, Stoter G, Oosterwijk E (2006) Treatment of metastatic renal cell carcinoma with autologous T-lymphocytes genetically retargeted against carbonic anhydrase IX: first clinical experience. J. Clin. Oncol. 24, e20 - e22. Maher J, Brentjens RJ, Gunset G, Rivière I, Sadelain M (2002) Human T-lymphocyte cytotoxicity and proliferation directed by a single chimeric TCRzeta /CD28 receptor. Nat Biotechnol. 20 (1), 70-75.

Maude SL, Barrett DM, Teachey DT, Grupp SA (2014) Managing Cytokine Release Syndrome Associated with Novel T Cell-Engaging Therapies. Cancer J. 20 (2), 119-122.

Milone MC, Fish JD, Carpenito C, Carroll RG, Binder GK, Teachey D, Samanta M, Lakhal M, Gloss B, Danet-Desnoyers G, Campana D, Riley JL, Grupp SA, June CH (2009) Chimeric receptors containing CD137 signal transduction domains mediate enhanced survival of T cells and increased antileukemic efficacy in vivo. Mol. Ther. 17 (8), 1453-1464.

Morgan RA, Yang JC, Kitano M, Dudley ME, Laurencot CM, Rosenberg SA (2010) Case Report of a Serious Adverse Event Following the Administration of T Cells Transduced With a Chimeric Antigen Receptor Recognizing ERBB2. Mol. Ther. 18, 843-851.

Pinthus JH, Waks T, Kaufman-Francis K, Schindler DG, Harmelin A, Kanety H, Ramon J, Eshhar Z (2003) Immuno-gene therapy of established prostate tumors using chimeric receptor-redirected human lymphocytes. Cancer Res. 63 (10), 2470-2476.

Pinthus JH, Waks T, Malina V, Kaufman-Francis K, Harmelin A, Aizenberg I, Kanety H, Ramon J, Eshhar Z (2004) Adoptive immunotherapy of prostate cancer bone lesions using redirected effector lymphocytes. J Clin Invest. 114(12), 1774-1781.

Prosser ME, Brown CE, Shami AF, Forman SJ, Jensen MC (2012) Tumor PD-L1 co-stimulates primary human CD8 cytotoxic T cells modified to express a PD1:CD28 chimeric receptor. Molecular Immunology 51 (3), 263-272.

Reinke AW, Grant RA, Keating AE (2010) A Synthetic Coiled-Coil Interactome Provides Heterospecific Modules for Molecular Engineering. JACS 132, 6025-6031.

Rafiq S, Yeku OO, Jackson HJ, Purdon TJ, van Leeuwen DG, Drakes DJ, Song M, Miele MM, Li Z, Wang P, Yan S, Xiang J, Ma X, Seshan VE, Henderickson RC, Liu C, Brentjens RJ (2018) Targeted delivery of a PD-1-blocking scFv by CAR-T cells enhances anti-tumor efficacy in vivo. Nature Biotechnology 36 (9), 847-856.

Sotillo E, Barrett DM, Black K., Bagashev A, Oldridge D, Wu G, Sussman R, Lanauze C, Ruella M, Gazzara MR, Martinez NM, Harrington CT, Chung EY, Perazzelli J, Hofmann TJ, Maude SL, Raman P, Barrera A, Gill S, Lacey SF, Melenhorst JJ, Allman D, Jacoby E, Fry T, Mackall C, Barash Y, Lynch KW, Maris JM, Grupp SA, Thomas-Tikhonenko A (2015) Convergence of Acquired Mutations and Alternative Splicing of CD19 Enables Resistance to CART-19 Immunotherapy. Cancer Discov. 5, 1282-1295.

Titov A, Petukhov A, Staliarova A, Motorin D, Bulatov E, Shuvalov O, Soond SM, Piacentini M, Melino G, Zaritskey A, Barlev NA (2018) The biological basis and clinical symptoms of CAR-T therapy-associated toxicites. Cell Death Dis 9, 897.

Töpfer K, Cartellieri M, Michen S, Wiedemuth R, Müller N, Lindemann D, Bachmann M, Füssel M, Schackert G, Temme A (2015) DAP12-based activating chimeric antigen receptor for NK cell tumor immunotherapy. J. Immunol. 194 (7), 3201-3212.

Wang J, Jensen M, Lin Y, Sui X, Chen E, Lindgren CG, Till B, Raubitschek A, Forman SJ, Qian X, James S, Greenberg P, Riddell S, Press OW (2007) Optimizing adoptive polyclonal T cell immunotherapy of lymphomas, using a chimeric T cell receptor possessing CD28 and CD137 costimulatory domains. Hum. Gene Ther. 18 (8), 712-725.

Yu S, Yi M, Qin S, Wu K (2019) Next generation chimeric antigen receptor T cells: safety strategies to overcome toxicity. Molecular Cancer. 18 (1).Zhang T, Lemoi BA, Sentman CL (2005) Chimeric NK-receptor-bearing T cells mediate antitumor immunotherapy. Blood. 106 (5), 1544-1551.

Zhang T, Barber A, Sentman CL (2006) Generation of antitumor responses by genetic modification of primary human T cells with a chimeric NKG2D receptor. Cancer Res. 66 (11) 5927-5933.

Zhang Y, Huo M, Zhou J, Xie S (2010) PKSolver: An add-in program for pharmacokinetic and pharmacodynamic data analysis in Microsoft Excel. Computer Methods and Programs in Biomedicine 99 (3), 306-314.

Zhao Y, Wang QJ, Yang S, Kochenderfer JN, Zheng Z, Zhong X, Sadelain M, Eshhar Z, Rosenberg SA, Morgan RA (2009) A herceptin-based chimeric antigen receptor with modified signaling domains leads to enhanced survival of transduced T lymphocytes and antitumor activity. J. Immunol. 183 (9), 5563-5574.

Reference signs

| | |
|---|---|
| 1 | first domain, a tag-binding domain or tag. |
| 2 | second domain, an extracellular hinge and a transmembrane domain. |
| 3 | third domain, a signal transduction domain. |
| 4 | optional fourth domain, a short peptide linker. |

1. A targeting module comprising
i) at least one PD-L1 and/or PD-L2 binding domain,
ii) a tag-binding domain or a tag,
for use in a method for stimulating a chimeric antigen receptor mediated immune response in a mammal, wherein the targeting module is administered in combination with a vector or a cell comprising a nucleotide sequence encoding a switchable chimeric antigen receptor, wherein the switchable chimeric antigen receptor comprises three domains, wherein
the first domain is a tag-binding domain or a tag,
the second domain is an extracellular hinge and a transmembrane domain and
the third domain is a signal transduction domain,
wherein the tag-binding domain of the targeting module binds to the tag of the switchable chimeric antigen receptor or the tag of the targeting module binds to the tag-binding domain of the switchable chimeric antigen receptor.

2. The targeting module according to claim 1 is monovalent or bivalent.

3. The targeting module according to claim 1 or 2, wherein the PD-L1 and/or PD-L2 binding domain is BMS-936559, Atezolizumab, Durvalumab, Avelumab, 3F2.1 or a fragment thereof.

4. The targeting module according to one of the claims 1 to 3, wherein the tag is a peptide epitope tag.

5. The targeting module according to claim 4, wherein the peptide epitope tag is a myc-tag, a His-tag, a short linear peptide sequence from yeast transcription factor GCN4, a leucine zipper sequence or a short linear peptide sequence from a human nuclear protein, preferably from the human La protein.

6. The targeting module according to one of the claims 1 to 5 further comprising an effector cell binding domain, wherein the effector cell binding domain specifically binds an epitope on a human CD3 chain, a human T cell receptor (TCR) or human CD16.

7. The targeting module according to one of the claims 1 to 6, wherein the variable regions of the at least one PD-L1 and/or PD-L2 binding domain and/or the effector cell binding domain comprise a humanized amino acid sequence.

8. The targeting module according to one of the claims 1 to 7, wherein the length is in the range of 20 to 1600 amino acids.

9. The targeting module according to one of the claims 1 to 8 for use in a method for stimulating a chimeric antigen receptor mediated immune response in a mammal,
wherein the targeting module is administered in combination with a vector or a cell comprising a nucleotide sequence encoding a switchable chimeric antigen receptor and at least one further targeting module,
wherein the at least one further targeting module comprises at least one target cell binding domain and a tag-binding domain or a tag,
wherein the at least one target cell binding domain is an antibody, antibody fragment, a protein, a peptide or a low molecular weight organic ligand that binds to surface antigens selected from the group comprising CD2, CD3, CD4, CD8, CD10, CD19, CD20, CD22, CD23, CD25, CD30, CD33, CD38, CD44, CD44v6 CD52, CD90, CD99, CD123, CD133, CD150 CD181, CD182, CD184, CD223, CD229, CD269 (BCMA), CD273, CD274, CD276, CD279, CD319, CD366 and CD371, interleukin receptors, especially preferred IL-8Rα (CXCR1), IL-8Rβ (CXCR2), IL-11Rα, IL-11Rβ, IL-13Rα1 and 2, CXCR4, c-Met, mesothelin, members of the epidermal growth factor receptor family and mutants thereof, especially preferred ErbB1, ErbB2, ErbB3, ErbB4 or mutants thereof, members of the tumor necrosis factor receptor superfamily, ephrins, ephrin receptors, especially preferred EphA1-10, EphA5 or EphB1-6, prostate specific antigens prostate stem cell antigen (PSCA) and prostate specific membrane antigen (PSMA), embryonic antigens carcinoembryonic antigen (CEA) and fetal acethylcholine receptor, members of the vascular endothelia growth factor family, epithelia cell adhesion molecule EpCAM, alphafetoprotein AFP, members of the intercellular adhesion molecule family, members of the mucin protein family, follicle stimulating hormone receptor (FSHR), the human high molecular weight-melanoma-associated antigen (HMW-MAA), folate binding protein FBP, folate receptors, somatostatin receptors, ligands of the NKG2D receptor, cytokine receptors, members of the epithelia glycoprotein family, diasialogangliosides, glypicans, G protein-coupled receptors, members of the carbonic anhydrase family, members of the carbohydrate antigen family, Notch ligands, melanoma-associated chondroitin sulfate proteoglycan (MCSP), glycoprotein A33, guanylatecyclase 2C and tumor-specific glycans, including mutants and analogues of the named antibodies, antibody fragments, proteins, peptides or low molecular weight organic ligands,
wherein the targeting module according to one of the claims 1 to 8 and the at least one further targeting module comprise different target cell binding domains, and identical tag-binding domains or a tags.

10. A nucleic acid, a vector or a cell comprising a nucleotide sequence encoding a targeting module according to one of the claims 1 to 9 for use in a method for stimulating a chimeric antigen receptor mediated immune response in a mammal,
wherein the nucleic acid, vector or cell is administered in combination with a vector or a cell comprising a nucleotide sequence encoding a switchable chimeric antigen receptor,
wherein the switchable chimeric antigen receptor comprises three domains,
wherein
the first domain is a tag-binding domain or a tag,
the second domain is an extracellular hinge and a transmembrane domain and
the third domain is a signal transduction domain,
wherein the tag-binding domain of the targeting module binds to the tag of the switchable chimeric antigen receptor or the tag of the targeting module binds to the tag-binding domain of the switchable chimeric antigen receptor.

11. The nucleic acid, vector or cell according to claim 10 further comprising an inducible expression system, preferably selected from Tet-On, Tet-On Advanced, Tet-On 3G, T-REx or a promoter responsive to nuclear factor of activated T cells (NFAT).

12. The nucleic acid, vector or cell according to claim 10 or 11 further comprising a nucleotide sequence encoding a switchable chimeric antigen receptor,
wherein the switchable chimeric antigen receptor comprises three domains,
wherein
the first domain is a tag-binding domain or tag,
the second domain is an extracellular hinge and a transmembrane domain and
the third domain is a signal transduction domain,
wherein the tag-binding domain of the targeting module binds to the tag of the switchable chimeric antigen receptor or the tag of the targeting module binds to the tag-binding domain of the switchable chimeric antigen receptor.

13. A nucleic acid, a vector or a cell comprising
an inducible expression system, preferably selected from Tet-On, Tet-On Advanced, Tet-On 3G, TREx or a promoter responsive to NFAT, and
a nucleotide sequence encoding a switchable chimeric antigen receptor, wherein the switchable chimeric antigen receptor comprises three domains,
wherein
the first domain is a tag-binding domain or tag,
the second domain is an extracellular hinge and a transmembrane domain and
the third domain is a signal transduction domain, and
a nucleotide sequence encoding a targeting module according to one of the claims 1 to 9,
wherein the tag-binding domain of the targeting module binds to the tag of the switchable chimeric antigen receptor or the tag of the targeting module binds to the tag-binding domain of the switchable chimeric antigen receptor.

14. A pharmaceutical composition comprising the targeting module according to one of the claims 1 to 9, the nucleic acid, vector or cell according to one of the claims 10 to 13 for use in a
method for stimulating a chimeric antigen receptor mediated immune response in a mammal,
wherein the pharmaceutical composition is administered in combination with a vector or a cell comprising a nucleotide sequence encoding a switchable chimeric antigen receptor,
wherein the switchable chimeric antigen receptor comprises three domains,
wherein
the first domain is a tag-binding domain or a tag,
the second domain is an extracellular hinge and a transmembrane domain and
the third domain is a signal transduction domain,
wherein the tag-binding domain of the targeting module binds to the tag of the switchable chimeric antigen receptor or the tag of the targeting module binds to the tag-binding domain of the switchable chimeric antigen receptor.

15. A kit comprising
a) a targeting module according to one of the claims 1 to 9, the nucleic acid, vector or cell according to one of the claims 10 to 13 and
b) a vector or a cell comprising a nucleotide sequence encoding a switchable chimeric antigen receptor,
wherein the switchable chimeric antigen receptor comprises three domains,
wherein
the first domain is a tag-binding domain or tag,
the second domain is an extracellular hinge and a transmembrane domain and
the third domain is a signal transduction domain,
wherein the tag-binding domain of the targeting module binds to the tag of the switchable chimeric antigen receptor or the tag of the targeting module binds to the tag-binding domain of the switchable chimeric antigen receptor.

16. The kit according to claim 15, wherein the tag is a myc-tag, a His-tag, a short linear peptide sequence from yeast transcription factor GCN4, a leucine zipper sequence or a short linear peptide sequence from a human nuclear protein, preferably from the human La protein.

17. The kit according to claim 15 or 16, wherein the tag-binding domain is an antibody or an antibody fragment binding to a myc-tag, a His-tag, a short linear peptide sequence from yeast transcription factor GCN4, a leucine zipper sequence or a short linear peptide sequence from a human nuclear protein, preferably from a human La protein.

18. The kit according to one of the claims 15 to 17, wherein the extracellular hinge and transmembrane domain is selected from hinge and transmembrane domains of human CD28 molecule, CD8a chain NK cell receptors, preferably natural killer group NKG2D; or parts of the constant region of an antibody and combinations thereof.

19. The kit according to one of the claims 15 to 18, wherein the signal transduction domain is selected from cytoplasmic regions of CD28, CD137 (4-1BB), CD134 (OX40), CD278 (ICOS), DAP10 and CD27, programmed cell death-1 (PD-1), cytotoxic T-lymphocyte antigen 4 (CTLA-4), cytoplasmic regions of CD3 chains, DAP12, CD122 (interleukin-2 receptor β), CD132 (interleukin-2 receptor γ), CD127 (interleukin-7 receptor α), CD360 (interleukin-21 receptor), activating Fc receptors and mutants thereof.

20. The kit according to one of the claims 15 to 19, wherein the targeting module and/or the vector or cell comprising a nucleotide sequence encoding a switchable chimeric antigen receptor are in the form of a pharmaceutical composition.

21. The kit according to one of the claims 15 to 20 for use in a method for stimulating a chimeric antigen receptor mediated immune response in a mammal, preferably for use in the treatment of cancer, infectious disease or autoimmune disease.


SEQUENCE LISTING (108 sequences)

SEQ ID NO: 1 - protein, 272 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 254 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 3 - protein, 265 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 4 - protein, 123 aa, Artificial Sequence
  Humanized VH chain
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 5 - protein, 106 aa, Artificial Sequence
  Humanized VL chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 6 - protein, 118 aa, Artificial Sequence [sequence omitted]

SEQ ID NO: 7 - protein, 107 aa, Artificial Sequence [sequence omitted]

SEQ ID NO: 8 - protein, 121 aa, Artificial Sequence
  Humanized VH chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 9 - protein, 108 aa, Artificial Sequence
  Humanized VL chain
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 10 - protein, 33 aa, Saccharomyces cerevisiae
RMKQLEPKVEELLPKNYHLENEVARLKKLVGER

SEQ ID NO: 11 - protein, 14 aa, Saccharomyces cerevisiae
NYHLENEVARLKKL

SEQ ID NO: 12 - protein, 51 aa, Homo sapiens
MDPDLEIRAAFLRQRNTALRTEVAELEQEVQRLENEVSQYETRYGPLGGGK

SEQ ID NO: 13 - protein, 51 aa, Homo sapiens
MDPDLEIEAAFLERENTALETRVAELRQRVQRLRNRVSQYRTRYGPLGGGK

SEQ ID NO: 14 - protein, 10 aa, Homo sapiens
KPLPEVTDEY

SEQ ID NO: 15 - protein, 20 aa, Homo sapiens
  MISC_FEATURE (16)..(16): Xaa can be alanine or serine
LEKEALKKIIEDQQEXLNKW

SEQ ID NO: 16 - protein, 20 aa, Homo sapiens
LEKEALKKIIEDQQEALNKW

SEQ ID NO: 17 - protein, 113 aa, Artificial Sequence
  Humanized scFv VL chain
  MISC_FEATURE (24)..(24): Xaa can be any naturally occurring amino acid
  MISC_FEATURE (35)..(35): Xaa can be any naturally occurring amino acid
  MISC_FEATURE (61)..(61): Xaa can be any naturally occurring amino acid
  MISC_FEATURE (101)..(101): Xaa can be any naturally occurring amino acid
DIVMTQSPDSLAVSLGERATINCXSSQSLLNSRTXKNYLAWYQQKPGQPPKLLIYWASTRXSGVPDRFSGSGSGTDFTLTISSLQAEDVAVYYCKQSYNLXTFGGGTKVEILK

SEQ ID NO: 18 - protein, 120 aa, Artificial Sequence
  Humanized scFv VH chain
QVQLVQSGAEVKKPGASVKVSCKASGYTFTHYYIYWVRQAPGQGLEWMGGVNPSNGGTHFNEKFKSRVTMTRDTSISTAYMELSRLRSDDTAVYYCARSEYDYGLGFAYWGQGTLVTVSS

SEQ ID NO: 19 - protein, 112 aa, Artificial Sequence
  Humanized scFv VL chain
DIVMTQSPDSLAVSLGERATINCKSSQSLLNSRTPKNYLAWYQQKPGQPPKLLIYWASTRKSGVPDRFSGSGSGTDFTLTISSLQAEDVAVYYCKQSYNLLTFGGGTKVEIK

SEQ ID NO: 20 - protein, 118 aa, Artificial Sequence
  Humanized scFv VH chain
EVQLVESGGGLVQPGGSLRLSCAASGFTFSDFWMNWVRQAPGKGLEWVGQIRNKPNNYETYYSDSLKGRFTISRDDSKSITYLQMNSLRAEDTAVYYCTLGNSWFAYWGQGTLVTVSS

SEQ ID NO: 21 - protein, 112 aa, Artificial Sequence
  Humanized scFv VL chain
DIVMTQSPDSLAVSLGERATINCRSSQSLLDSRTKKNYLAWYQQKPGQPPKLLIYWASTRESGVPDRFSGSGSGTDFTLTISSLQAEDVAVYYCKQSYNLPTFGGGTKVEIK

SEQ ID NO: 22 - protein, 115 aa, Mus musculus
DIVLTQTPAIMSASPGEKVTMTCSASSSVSYMHWYQQKSGTSPKRWIYDTSKLASGVPTRFSGSGSGTSYSLTISSMEAEDAATYYCQQWSSNPLTFGAGTKLELKRADAAPTVS

SEQ ID NO: 23 - protein, 117 aa, Mus musculus
EVKLQESGPELVKSGASVKMSCKASGYTFTKYVIHWVKQKPGQGLEWIGYINPYNDVSKYNEKFRGKATLTSDKSSSTAYMELSSLTSEDSAVYFCSRNKDYYPMDYWGQGTSVTVS

SEQ ID NO: 24 - protein, 105 aa, Artificial Sequence
  Humanized scFv VL chain
EIVLTQSPATLSLSPGERATLSCSASSSVSYMHWYQQKPGQAPRLLIYDTSKLASGIPARFSGSGSGTDFTLTISSLEPEDFAVYYCQQWSSNPLTFGGGTKVEI

SEQ ID NO: 25 - protein, 117 aa, Artificial Sequence
  Humanized scFv VH chain
QVQLVQSGAEVKKPGASVKVSCKASGYTFTKYVIHWVRQAPGQGLEWMGYINPYNDVSKYNEKFRGRVTMTTDTSTSTAYMELRSLRSDDTAVYYCARNKDYYPMDYWGQGTTVTVS

SEQ ID NO: 26 - protein, 108 aa, Artificial Sequence
  Humanized antibody VL chain
YELIQPSSASVTVGETVKITCSGDQLPKNFAYWFQQKSDKNILLLIYMDNKRPSGIPERFSGSTSGTTATLTISGAQPEDEAAYYCLSSYGDNNDLVFGSGTQLTVLR

SEQ ID NO: 27 - protein, 121 aa, Artificial Sequence
  Humanized antibody VH chain
EVYLVESGGDLVQPGSSLKVSCAASGFTFSDFWMYWVRQAPGKGLEWVGRIKNIPNNYATEYADSVRGRFTISRDDSRNSIYLQMNRLRVDDTAIYYCTRAGRFDHFDYWGQGTMVTVSSA

SEQ ID NO: 28 - protein, 106 aa, Artificial Sequence
  Humanized antibody VL chain
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 29 - protein, 120 aa, Artificial Sequence
  Humanized antibody VH chain
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 30 - protein, 335 aa, Artificial Sequence
  Fusion protein Escherichia coli and herpes simplex virus
MSRLDKSKVINSALELLNEVGIEGLTTRKLAQKLGVEQPTLYWHVKNKRALLDALAIEMLDRHHTHFCPLKGESWQDFLRNNAKSFRCALLSHRNGAKVHSDTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 31 - protein, 248 aa, Artificial Sequence
  Fusion protein Escherichia coli and herpes simplex virus
MSRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 32 - protein, 248 aa, Artificial Sequence
  Fusion protein Escherichia coli and herpes simplex virus
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 33 - DNA, 19 bp, Escherichia coli
tccctatcagtgatagaga

SEQ ID NO: 34 - DNA, 16 bp, Artificial Sequence
  Spacer sequence
acgatgtcgagtttac

SEQ ID NO: 35 - DNA, 379 bp, Artificial Sequence
  TRE sequence
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 36 - DNA, 30 bp, Homo sapiens
ggaggaaaaactgtttcatacagaaggcgt

SEQ ID NO: 37 - DNA, 52 bp, Homo sapiens
cagaattaacagtataaattgcatctcttgttcaagagttccctatcactct

SEQ ID NO: 38 - DNA, 110 bp, Homo sapiens
acattttgacacccccataatatttttccagaattaacagtataaattgcatctcttgttcaagagttccctatcactctctttaatcactactcacagtaacctcaact

SEQ ID NO: 39 - DNA, 232 bp, Artificial Sequence
  Fusion of human DNA sequences
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtcagaattaacagtataaattgcatctcttgttcaagagttccctatcactct

SEQ ID NO: 40 - DNA, 296 bp, Artificial Sequence
  Fusion of human DNA sequences
aggaggaaaaactgtttcatacagaaggcgtaggaggaaaaactgtttcatacagaaggcgtaggaggaaaaactgtttcatacagaaggcgtaggaggaaaaactgtttcatacagaaggcgtaggaggaaaaactgtttcatacagaaggcgtaggaggaaaaactgtttcatacagaaggcgtacattttgacacccccataatatttttccagaattaacagtataaattgcatctcttgttcaagagttccctatcactctctttaatcactactcacagtaacctcaact

SEQ ID NO: 41 - protein, 627 aa, Artificial Sequence
  humanized homo sapiens/mus musculus chimera
MRRMQLLLLIALSLALVTNSEFQVQLVQSGAEVKKPGASVKVSCKASGYTFTHYYIYWVRQAPGQGLEWMGGVNPSNGGTHFNEKFKSRVTMTRDTSISTAYMELSRLRSDDTAVYYCARSEYDYGLGFAYWGQGTLVTVSSGGGGSGGGGSGGGGSDIVMTQSPDSLAVSLGERATINCKSSQSLLNSRTPKNYLAWYQQKPGQPPKLLIYWASTRKSGVPDRFSGSGSGTDFTLTISSLQAEDVAVYYCKQSYNLLTFGGGTKVEIKAAALEKEALKKIIEDQQEALNKWAAAGPGGGGSKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMAPPPALDNEKSNGTIIHVKGKHLCPSPLFPGPSKPFWVLVVVGGVLACYSLLVTVAFIIFWVRSKRSRGGHSDYMNMTPRRPGPTRKHYQPYAPPRDFAAYRSGGGGSGGGASGGGGSFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYGALHMQALPPR

SEQ ID NO: 42 - protein, 532 aa, Artificial Sequence
  humanized homo sapiens/mus musculus chimera
MRRMQLLLLIALSLALVTNSEFQVQLVQSGAEVKKPGASVKVSCKASGYTFTHYYIYWVRQAPGQGLEWMGGVNPSNGGTHFNEKFKSRVTMTRDTSISTAYMELSRLRSDDTAVYYCARSEYDYGLGFAYWGQGTLVTVSSGGGGSGGGGSGGGGSDIVMTQSPDSLAVSLGERATINCKSSQSLLNSRTPKNYLAWYQQKPGQPPKLLIYWASTRKSGVPDRFSGSGSGTDFTLTISSLQAEDVAVYYCKQSYNLLTFGGGTKVEIKAAALEKEALKKIIEDQQEALNKWAAAGPGGGGSTTTPAPRPPTPAPTIASQPLSLRPEACRPAAGGAVHTRGLDFACDIYIWAPLAGTCGVLLLSLVITLYCGGGGSKRGRKKLLYIFKQPFMRPVQTTQEEDGCSCRFPEEEEGGCELGGGGSFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 615 aa, Artificial Sequence
  humanized homo sapiens/mus musculus chimera
MRRMQLLLLIALSLALVTNSEFEVQLVESGGGLVQPGGSLRLSCAASGFTFSDFWMNWVRQAPGKGLEWVGQIRNKPNNYETYYSDSLKGRFTISRDDSKSITYLQMNSLRAEDTAVYYCTLGNSWFAYWGQGTLVTVSSGGGGSGGGGSGGGGSDIVMTQSPDSLAVSLGERATINCRSSQSLLDSRTKKNYLAWYQQKPGQPPKLLIYWASTRESGVPDRFSGSGSGTDFTLTISSLQAEDVAVYYCKQSYNLPTFGGGTKVEIKAAAKPLPEVTDEYAAAGPGGGGSKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMAPPPALDNEKSNGTIIHVKGKHLCPSPLFPGPSKPFWVLVVVGGVLACYSLLVTVAFIIFWVRSKRSRGGHSDYMNMTPRRPGPTRKHYQPYAPPRDFAAYRSGGGGSGGGASGGGGSFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYGALHMQALPPR

SEQ ID NO: 44 - protein, 520 aa, Artificial Sequence
  humanized homo sapiens/mus musculus chimera
MRRMQLLLLIALSLALVTNSEFEVQLVESGGGLVQPGGSLRLSCAASGFTFSDFWMNWVRQAPGKGLEWVGQIRNKPNNYETYYSDSLKGRFTISRDDSKSITYLQMNSLRAEDTAVYYCTLGNSWFAYWGQGTLVTVSSGGGGSGGGGSGGGGSDIVMTQSPDSLAVSLGERATINCRSSQSLLDSRTKKNYLAWYQQKPGQPPKLLIYWASTRESGVPDRFSGSGSGTDFTLTISSLQAEDVAVYYCKQSYNLPTFGGGTKVEIKAAAKPLPEVTDEYAAAGPGGGGSTTTPAPRPPTPAPTIASQPLSLRPEACRPAAGGAVHTRGLDFACDIYIWAPLAGTCGVLLLSLVITLYCGGGGSKRGRKKLLYIFKQPFMRPVQTTQEEDGCSCRFPEEEEGGCELGGGGSFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 367 aa, Artificial Sequence
  humanized homo sapiens/mus musculus chimera
MRRMQLLLLIALSLALVTNSEFKPLPEVTDEYAAAGPGGGGSKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMAPPPALDNEKSNGTIIHVKGKHLCPSPLFPGPSKPFWVLVVVGGVLACYSLLVTVAFIIFWVRSKRSRGGHSDYMNMTPRRPGPTRKHYQPYAPPRDFAAYRSGGGGSGGGASGGGGSFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYGALHMQALPPR

SEQ ID NO: 46 - protein, 379 aa, Artificial Sequence
  humanized homo sapiens/mus musculus chimera
MRRMQLLLLIALSLALVTNSEFKPLPEVTDEYAAAGPGGGGSKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMAPPPALDNEKSNGTIIHVKGKHLCPSPLFPGPSKPFWVLVVVGGVLACYSLLVTVAFIIFWVRSKRSRGGGGSKRGRKKLLYIFKQPFMRPVQTTQEEDGCSCRFPEEEEGGCELGGGGSGGGASGGGGSFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYGALHMQALPPR

SEQ ID NO: 47 - protein, 375 aa, Artificial Sequence
  humanized homo sapiens/mus musculus chimera
MRRMQLLLLIALSLALVTNSEFEKEALKKIIEDQQESLNKAAAGPGGGGSKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMAPPPALDNEKSNGTIIHVKGKHLCPSPLFPGPSKPFWVLVVVGGVLACYSLLVTVAFIIFWVRSKRSRGGHSDYMNMTPRRPGPTRKHYQPYAPPRDFAAYRSGGGGSGGGASGGGGSFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYGALHMQALPPR

SEQ ID NO: 48 - protein, 387 aa, Artificial Sequence
  humanized homo sapiens/mus musculus chimera
MRRMQLLLLIALSLALVTNSEFEKEALKKIIEDQQESLNKAAAGPGGGGSKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMAPPPALDNEKSNGTIIHVKGKHLCPSPLFPGPSKPFWVLVVVGGVLACYSLLVTVAFIIFWVRSKRSRGGGGSKRGRKKLLYIFKQPFMRPVQTTQEEDGCSCRFPEEEEGGCELGGGGSGGGASGGGGSFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYGALHMQALPPR

SEQ ID NO: 49 - DNA, 1881 bp, Artificial Sequence
  humanized homo sapiens/mus musculus chimera
atgcgccgcatgcagctgctgcttctgatcgctctgagcctggctcttgtgaccaactctgaattccaggtgcagctggtgcagagcggagccgaggtgaagaagcctggagcctctgtgaaggtgagctgcaaggcttctggctacaccttcacccactactacatctactgggtgagacaggctcccggacagggcctggagtggatgggaggcgtgaaccccagcaacggaggcacccacttcaacgagaagttcaagtctcgcgtgaccatgacccgcgacaccagcatctctaccgcttacatggagctgagccgcctgcgctctgatgataccgctgtgtactactgcgctcgcagcgagtacgattacggactgggcttcgcctactggggccagggaaccctggtgaccgtgagctctggaggcggaggcagcggaggcggcggatctggaggcggaggaagcgatatcgtgatgacccagtctcctgatagcctggctgtgagcctgggcgagagagctaccatcaactgcaagagcagccagagcctgctgaactctcgcacccctaagaactaccttgcttggtaccagcagaagcctggacagccccctaagctgctgatctactgggcttctacccgcaagagcggcgtgcccgacagattctctggcagcggaagcggcaccgatttcaccctgaccatcagcagcctgcaggctgaggacgtggccgtgtactactgcaagcagtcttacaacctgctgaccttcggaggcggaaccaaggtggagatcaaggctgccgctctggagaaggaggccctgaagaagatcatcgaggatcagcaggaggctctgaacaagtgggctgccgctgggcccggaggaggcggcagcaagatcctggtcaaacagtcccctatgctggtcgcttacgacaacgccgttaatctgagttgcaaatatagttacaacctgtttagccgggaatttcgcgcatctctccacaagggactggattctgcggttgaggtttgtgtggtctatggcaattatagccagcaactgcaagtgtacagcaaaacaggctttaactgcgacgggaaactcgggaacgaatcagtgaccttctatctgcagaacctgtacgttaaccaaacagatatttacttctgcaagatagaggtgatggctccaccgccagcactggataacgagaagtccaatggaaccatcattcacgtcaaggggaagcatctgtgtccttccccgttgttccctgggccgagcaaacccttttgggtgcttgtggtagttggcggggtattggcctgctattcccttctcgtaactgtggccttcatcatcttctgggtcagatctaagaggtctaggggcgggcatagcgactacatgaacatgacacccaggcggcctggccccactcgcaaacactaccagccatacgcaccaccaagagactttgccgcatatcggagtggtggcggcgggtcaggaggtggagctagcggtggaggaggttccttctctaggtcagctgatgctcccgcctatcagcaaggtcagaaccagctctacaatgagctgaatctgggacgtcgggaggagtacgacgtgctggataaacgaagaggacgcgatcccgagatgggtgggaagcctaggcgcaagaatccccaggaaggcctctacaatgaactgcagaaagacaagatggccgaagcctacagcgagattggcatgaaaggggagcgacggagaggaaagggacatgacgggttgtatcagggtctttccactgcgacaaaggatacctatggggctctgcacatgcaagcactgccacctaga

SEQ ID NO: 50 - DNA, 1596 bp, Artificial Sequence
  humanized homo sapiens/mus musculus chimera
atgcgccgcatgcagctgctgcttctgatcgctctgagcctggctcttgtgaccaactctgaattccaggtgcagctggtgcagagcggagccgaggtgaagaagcctggagcctctgtgaaggtgagctgcaaggcttctggctacaccttcacccactactacatctactgggtgagacaggctcccggacagggcctggagtggatgggaggcgtgaaccccagcaacggaggcacccacttcaacgagaagttcaagtctcgcgtgaccatgacccgcgacaccagcatctctaccgcttacatggagctgagccgcctgcgctctgatgataccgctgtgtactactgcgctcgcagcgagtacgattacggactgggcttcgcctactggggccagggaaccctggtgaccgtgagctctggaggcggaggcagcggaggcggcggatctggaggcggaggaagcgatatcgtgatgacccagtctcctgatagcctggctgtgagcctgggcgagagagctaccatcaactgcaagagcagccagagcctgctgaactctcgcacccctaagaactaccttgcttggtaccagcagaagcctggacagccccctaagctgctgatctactgggcttctacccgcaagagcggcgtgcccgacagattctctggcagcggaagcggcaccgatttcaccctgaccatcagcagcctgcaggctgaggacgtggccgtgtactactgcaagcagtcttacaacctgctgaccttcggaggcggaaccaaggtggagatcaaggctgccgctctggagaaggaggccctgaagaagatcatcgaggatcagcaggaggctctgaacaagtgggctgccgctgggcccggaggaggcgggtcaaccactacccctgctcccaggcctcctacacccgcgccaacgattgccagtcaacctctgtccctgaggcctgaagcctgcagaccagctgcaggcggtgctgtgcatacacggggcttggactttgcctgcgatatctacatctgggcacctcttgccgggacttgtggcgttctcctgctttctctggtcattaccctgtactgtggaggcggcggtagcaaacgcggacggaagaaactgctgtacatcttcaagcagcccttcatgcgccccgtgcagacaacacaggaagaggacggttgcagctgccgatttcccgaagaggaggagggaggctgtgaattgggcggaggaggctccttctctcgcagtgcagatgccccagcgtacaagcaaggccagaaccagctgtacaacgagctcaatctggggcgtagagaggagtatgacgtgctggacaagaggcgaggaagggaccccgagatgggtggcaaaccgcggcgtaagaatccgcaggaaggcctctacaacgagctgcagaaagacaagatggccgaagcctattccgagatagggatgaaaggggaaagacggcgcggtaaagggcacgatgggctctatcagggtctgagcactgcaaccaaggacacgtatgatgccttgcacatgcaagctcttccaccaaga

SEQ ID NO: 51 - DNA, 1845 bp, Artificial Sequence
  humanized homo sapiens/mus musculus chimera
atgcgccgcatgcagctgctgcttctgatcgctctgagcctggctcttgtgaccaactctgaattcgaggtgcaactggtcgaaagtggcggtggtttagttcagcctggtggaagtctacggcttagctgcgcagcatccggtttcacctttagcgacttttggatgaactgggttcggcaggctccgggcaaaggactggagtgggttgggcaaatccgcaacaaaccgaataactacgaaacttattactcagatagcctgaagggtcgattcaccatcagcagggatgattcaaagtcaatcacttacctacagatgaactcattaagagcggaggatactgcggtgtattactgtacactaggtaactcctggttcgcgtattggggacagggcacccttgtaaccgtctccagcggaggcggaggcagcggaggcggcggatctggaggcggaggaagcgacattgttatgacccagagcccggactctctcgctgttagtcttggtgagcgagcgactattaactgccggagcagtcagagtttgttggactctcggacgaaaaagaactacctggcatggtaccagcagaagccgggccaaccacctaaattactgatatattgggcgtcgactcgtgagtcaggggtaccggacaggttttctggaagcggatcaggaacagacttcactttgacgatctcttcgcttcaagccgaggacgttgcggtttattattgtaagcaaagctataatctgccgacatttggtggcggcaccaaggttgaaattaaggctgccgctaagcctctgcctgaagtgaccgatgagtatgctgccgctgggcccggaggaggcggcagcaagatcctggtcaaacagtcccctatgctggtcgcttacgacaacgccgttaatctgagttgcaaatatagttacaacctgtttagccgggaatttcgcgcatctctccacaagggactggattctgcggttgaggtttgtgtggtctatggcaattatagccagcaactgcaagtgtacagcaaaacaggctttaactgcgacgggaaactcgggaacgaatcagtgaccttctatctgcagaacctgtacgttaaccaaacagatatttacttctgcaagatagaggtgatggctccaccgccagcactggataacgagaagtccaatggaaccatcattcacgtcaaggggaagcatctgtgtccttccccgttgttccctgggccgagcaaacccttttgggtgcttgtggtagttggcggggtattggcctgctattcccttctcgtaactgtggccttcatcatcttctgggtcagatctaagaggtctaggggcgggcatagcgactacatgaacatgacacccaggcggcctggccccactcgcaaacactaccagccatacgcaccaccaagagactttgccgcatatcggagtggtggcggcgggtcaggaggtggagctagcggtggaggaggttccttctctaggtcagctgatgctcccgcctatcagcaaggtcagaaccagctctacaatgagctgaatctgggacgtcgggaggagtacgacgtgctggataaacgaagaggacgcgatcccgagatgggtgggaagcctaggcgcaagaatccccaggaaggcctctacaatgaactgcagaaagacaagatggccgaagcctacagcgagattggcatgaaaggggagcgacggagaggaaagggacatgacgggttgtatcagggtctttccactgcgacaaaggatacctatggggctctgcacatgcaagcactgccacctaga

SEQ ID NO: 52 - DNA, 1563 bp, Artificial Sequence
  humanized homo sapiens/mus musculus chimera
atgcgccgcatgcagctgctgcttctgatcgctctgagcctggctcttgtgaccaactctgaattcgaggtgcaactggtcgaaagtggcggtggtttagttcagcctggtggaagtctacggcttagctgcgcagcatccggtttcacctttagcgacttttggatgaactgggttcggcaggctccgggcaaaggactggagtgggttgggcaaatccgcaacaaaccgaataactacgaaacttattactcagatagcctgaagggtcgattcaccatcagcagggatgattcaaagtcaatcacttacctacagatgaactcattaagagcggaggatactgcggtgtattactgtacactaggtaactcctggttcgcgtattggggacagggcacccttgtaaccgtctccagcggaggcggaggcagcggaggcggcggatctggaggcggaggaagcgacattgttatgacccagagcccggactctctcgctgttagtcttggtgagcgagcgactattaactgccggagcagtcagagtttgttggactctcggacgaaaaagaactacctggcatggtaccagcagaagccgggccaaccacctaaattactgatatattgggcgtcgactcgtgagtcaggggtaccggacaggttttctggaagcggatcaggaacagacttcactttgacgatctcttcgcttcaagccgaggacgttgcggtttattattgtaagcaaagctataatctgccgacatttggtggcggcaccaaggttgaaattaaggctgccgctaagcctctgcctgaagtgaccgatgagtatgctgccgctgggcccggaggaggcggcagcaccactacccctgctcccaggcctcctacacccgcgccaacgattgccagtcaacctctgtccctgaggcctgaagcctgcagaccagctgcaggcggtgctgtgcatacacggggcttggactttgcctgcgatatctacatctgggcacctcttgccgggacttgtggcgttctcctgctttctctggtcattaccctgtactgtggaggcggcggtagcaaacgcggacggaagaaactgctgtacatcttcaagcagcccttcatgcgccccgtgcagacaacacaggaagaggacggttgcagctgccgatttcccgaagaggaggagggaggctgtgaattgggcggaggaggctccttctctcgcagtgcagatgccccagcgtacaagcaaggccagaaccagctgtacaacgagctcaatctggggcgtagagaggagtatgacgtgctggacaagaggcgaggaagggaccccgagatgggtggcaaaccgcggcgtaagaatccgcaggaaggcctctacaacgagctgcagaaagacaagatggccgaagcctattccgagatagggatgaaaggggaaagacggcgcggtaaagggcacgatgggctctatcagggtctgagcactgcaaccaaggacacgtatgatgccttgcacatgcaagctcttccaccaagataa

SEQ ID NO: 53 - DNA, 1101 bp, Artificial Sequence
  humanized homo sapiens/mus musculus chimera
atgcgccgcatgcagctgctgcttctgatcgctctgagcctggctcttgtgaccaactctgaattcaaacccctacctgaagtgactgatgagtatgctgccgctgggcccggaggaggcggcagcaagatcctggtcaaacagtcccctatgctggtcgcttacgacaacgccgttaatctgagttgcaaatatagttacaacctgtttagccgggaatttcgcgcatctctccacaagggactggattctgcggttgaggtttgtgtggtctatggcaattatagccagcaactgcaagtgtacagcaaaacaggctttaactgcgacgggaaactcgggaacgaatcagtgaccttctatctgcagaacctgtacgttaaccaaacagatatttacttctgcaagatagaggtgatggctccaccgccagcactggataacgagaagtccaatggaaccatcattcacgtcaaggggaagcatctgtgtccttccccgttgttccctgggccgagcaaacccttttgggtgcttgtggtagttggcggggtattggcctgctattcccttctcgtaactgtggccttcatcatcttctgggtcagatctaagaggtctaggggcgggcatagcgactacatgaacatgacacccaggcggcctggccccactcgcaaacactaccagccatacgcaccaccaagagactttgccgcatatcggagtggtggcggcgggtcaggaggtggagctagcggtggaggaggttccttctctaggtcagctgatgctcccgcctatcagcaaggtcagaaccagctctacaatgagctgaatctgggacgtcgggaggagtacgacgtgctggataaacgaagaggacgcgatcccgagatgggtgggaagcctaggcgcaagaatccccaggaaggcctctacaatgaactgcagaaagacaagatggccgaagcctacagcgagattggcatgaaaggggagcgacggagaggaaagggacatgacgggttgtatcagggtctttccactgcgacaaaggatacctatggggctctgcacatgcaagcactgccacctaga

SEQ ID NO: 54 - DNA, 1137 bp, Artificial Sequence
  humanized homo sapiens/mus musculus chimera
atgcgccgcatgcagctgctgcttctgatcgctctgagcctggctcttgtgaccaactctgaattcaaacccctacctgaagtgactgatgagtatgctgccgctgggcccggaggaggcggcagcaagatcctggtcaaacagtcccctatgctggtcgcttacgacaacgccgttaatctgagttgcaaatatagttacaacctgtttagccgggaatttcgcgcatctctccacaagggactggattctgcggttgaggtttgtgtggtctatggcaattatagccagcaactgcaagtgtacagcaaaacaggctttaactgcgacgggaaactcgggaacgaatcagtgaccttctatctgcagaacctgtacgttaaccaaacagatatttacttctgcaagatagaggtgatggctccaccgccagcactggataacgagaagtccaatggaaccatcattcacgtcaaggggaagcatctgtgtccttccccgttgttccctgggccgagcaaacccttttgggtgcttgtggtagttggcggggtattggcctgctattcccttctcgtaactgtggccttcatcatcttctgggtcagatctaagaggtctaggggcgggggcgggtcaaaacgcggacggaagaaactgctgtacatcttcaagcagcccttcatgcgccccgtgcagacaacacaggaagaggacggttgcagctgccgatttcccgaagaggaggagggaggctgtgaattgggtggcggcgggtcaggaggtggagctagcggtggaggaggttccttctctaggtcagctgatgctcccgcctatcagcaaggtcagaaccagctctacaatgagctgaatctgggacgtcgggaggagtacgacgtgctggataaacgaagaggacgcgatcccgagatgggtgggaagcctaggcgcaagaatccccaggaaggcctctacaatgaactgcagaaagacaagatggccgaagcctacagcgagattggcatgaaaggggagcgacggagaggaaagggacatgacgggttgtatcagggtctttccactgcgacaaaggatacctatggggctctgcacatgcaagcactgccacctaga

SEQ ID NO: 55 - DNA, 1125 bp, Artificial Sequence
  humanized homo sapiens/mus musculus chimera
atgcgccgcatgcagctgctgcttctgatcgctctgagcctggctcttgtgaccaactctgaattcgagaaagaagcactgaagaaaataatagaagaccaacaagaatccctaaacaaagctgccgctgggcccggaggaggcggcagcaagatcctggtcaaacagtcccctatgctggtcgcttacgacaacgccgttaatctgagttgcaaatatagttacaacctgtttagccgggaatttcgcgcatctctccacaagggactggattctgcggttgaggtttgtgtggtctatggcaattatagccagcaactgcaagtgtacagcaaaacaggctttaactgcgacgggaaactcgggaacgaatcagtgaccttctatctgcagaacctgtacgttaaccaaacagatatttacttctgcaagatagaggtgatggctccaccgccagcactggataacgagaagtccaatggaaccatcattcacgtcaaggggaagcatctgtgtccttccccgttgttccctgggccgagcaaacccttttgggtgcttgtggtagttggcggggtattggcctgctattcccttctcgtaactgtggccttcatcatcttctgggtcagatctaagaggtctaggggcgggcatagcgactacatgaacatgacacccaggcggcctggccccactcgcaaacactaccagccatacgcaccaccaagagactttgccgcatatcggagtggtggcggcgggtcaggaggtggagctagcggtggaggaggttccttctctaggtcagctgatgctcccgcctatcagcaaggtcagaaccagctctacaatgagctgaatctgggacgtcgggaggagtacgacgtgctggataaacgaagaggacgcgatcccgagatgggtgggaagcctaggcgcaagaatccccaggaaggcctctacaatgaactgcagaaagacaagatggccgaagcctacagcgagattggcatgaaaggggagcgacggagaggaaagggacatgacgggttgtatcagggtctttccactgcgacaaaggatacctatggggctctgcacatgcaagcactgccacctaga

SEQ ID NO: 56 - DNA, 1161 bp, Artificial Sequence
  humanized homo sapiens/mus musculus chimera
atgcgccgcatgcagctgctgcttctgatcgctctgagcctggctcttgtgaccaactctgaattcgagaaagaagcactgaagaaaataatagaagaccaacaagaatccctaaacaaagctgccgctgggcccggaggaggcggcagcaagatcctggtcaaacagtcccctatgctggtcgcttacgacaacgccgttaatctgagttgcaaatatagttacaacctgtttagccgggaatttcgcgcatctctccacaagggactggattctgcggttgaggtttgtgtggtctatggcaattatagccagcaactgcaagtgtacagcaaaacaggctttaactgcgacgggaaactcgggaacgaatcagtgaccttctatctgcagaacctgtacgttaaccaaacagatatttacttctgcaagatagaggtgatggctccaccgccagcactggataacgagaagtccaatggaaccatcattcacgtcaaggggaagcatctgtgtccttccccgttgttccctgggccgagcaaacccttttgggtgcttgtggtagttggcggggtattggcctgctattcccttctcgtaactgtggccttcatcatcttctgggtcagatctaagaggtctaggggcgggggcgggtcaaaacgcggacggaagaaactgctgtacatcttcaagcagcccttcatgcgccccgtgcagacaacacaggaagaggacggttgcagctgccgatttcccgaagaggaggagggaggctgtgaattgggtggcggcgggtcaggaggtggagctagcggtggaggaggttccttctctaggtcagctgatgctcccgcctatcagcaaggtcagaaccagctctacaatgagctgaatctgggacgtcgggaggagtacgacgtgctggataaacgaagaggacgcgatcccgagatgggtgggaagcctaggcgcaagaatccccaggaaggcctctacaatgaactgcagaaagacaagatggccgaagcctacagcgagattggcatgaaaggggagcgacggagaggaaagggacatgacgggttgtatcagggtctttccactgcgacaaaggatacctatggggctctgcacatgcaagcactgccacctaga

SEQ ID NO: 57 - protein, 274 aa, Artificial Sequence
  Tagged humanized scFv
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKAAAKPLPEVTDEYARGGPGGGGSHHHHHH

SEQ ID NO: 58 - protein, 278 aa, Artificial Sequence
  Tagged humanized scFv
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSGGGGSGGGGSGGGGSGGGGSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKAAAKPLPEVTDEYARGGPGGGGSHHHHHH

SEQ ID NO: 59 - protein, 302 aa, Artificial Sequence
  Tagged human sequences
EFPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHARGKPLPEVTDEYAAAPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHAAAGGGGSHHHHHH

SEQ ID NO: 60 - protein, 532 aa, Artificial Sequence; identical to SEQ ID NO: 42
  humanized homo sapiens/mus musculus chimera

SEQ ID NO: 61 - protein, 450 aa, Artificial Sequence
  Humanized VH chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 216 aa, Artificial Sequence
  Humanized VL chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 63 - protein, 123 aa, Artificial Sequence
  Humanized VH chain
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSS

SEQ ID NO: 64 - protein, 107 aa, Artificial Sequence
  Humanized VL chain
DIQMTQSPSSLSASVGDRVTITCRASQSISSYLNWYQQKPGKAPKLLIYVASSLQSGVPSRFSGSGSGTDFTLTISSLQPEDFATYYCQQSYSTPITFGQGTRLEIK

SEQ ID NO: 65 - protein, 279 aa, Artificial Sequence
  Tagged humanized scFv
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGASAAGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSAAAKPLPEVTDEYARGGPGGGGSHHHHHH

SEQ ID NO: 66 - protein, 283 aa, Artificial Sequence
  Tagged humanized scFv
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKGGGGSGGGGSGASAAGGGGSGGGGSEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSAAAKPLPEVTDEYARGGPGGGGSHHHHHH

SEQ ID NO: 67 - protein, 117 aa, Artificial Sequence
  Humanized VH chain
QVQLVQSGAEVKKPGASVKVSCKASGYTFTDYGFSWVRQAPGQGLEWMGWITAYNGNTNYAQKLQGRVTMTTDTSTSTVYMELRSLRSDDTAVYYCARDYFYGMDVWGQGTTVTVSS

SEQ ID NO: 68 - protein, 107 aa, Artificial Sequence
  Humanized VL chain
EIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIK

SEQ ID NO: 69 - protein, 118 aa, Artificial Sequence
  Humanized VH chain
QVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSS

SEQ ID NO: 70 - protein, 107 aa, Artificial Sequence
  Humanized VL chain
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 71 - protein, 123 aa, Artificial Sequence
  Humanized VH chain
QVQLVQSGAEVKEPGSSVKVSCKASGGTFNSYAISWVRQAPGQGLEWMGGIIPLFGIAHYAQKFQGRVTITADESTNTAYMDLSSLRSEDTAVYYCARKYSYVSGSPFGMDVWGQGTTVTVSS

SEQ ID NO: 72 - protein, 106 aa, Artificial Sequence
  Humanized VL chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTRLEIK

SEQ ID NO: 73 - protein, 121 aa, Artificial Sequence
  Humanized VH chain
QVQLVQSGAEVKKPGSSVKVSCKASGGTFSSYAINWVRQAPGQGLEWMGGIIPIFGSANYAQKFQDRVTITADESTSAAYMELSSLRSEDTAVYYCARDSSGWSRYYMDVWGQGTTVTVSS

SEQ ID NO: 74 - protein, 106 aa, Artificial Sequence
  Humanized VL chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFGGGTKVEIK

SEQ ID NO: 75 - protein, 120 aa, Artificial Sequence
  Humanized VH chain
QVQLVQSGAEVKKPGSSVKVSCKVSGGIFSTYAINWVRQAPGQGLEWMGGIIPIFGTANHAQKFQGRVTITADESTSTAYMELSSLRSEDTAVYYCARDQGIAAALFDYWGQGTLVTVSS

SEQ ID NO: 76 - protein, 108 aa, Artificial Sequence
  Humanized VL chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 77 - protein, 121 aa, Artificial Sequence
  Humanized VH chain
EVQLVESGGGLVQPGRSLRLSCAASGITFDDYGMHWVRQAPGKGLEWVSGISWNRGRIEYADSVKGRFTISRDNAKNSLYLQMNSLRAEDTALYYCAKGRFRYFDWFLDYWGQGTLVTVSS

SEQ ID NO: 78 - protein, 107 aa, Artificial Sequence
  Humanized VL chain
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIK

SEQ ID NO: 79 - protein, 113 aa, Artificial Sequence
  Humanized VH chain
EVQLVESGGGLVQPGRSLRLSCAVSGFTFDDYVVHWVRQAPGKGLEWVSGISGNSGNIGYADSVKGRFTISRDNAKNSLYLQMNSLRAEDTALYYCAVPFDYWGQGTLVTVSS

SEQ ID NO: 80 - protein, 107 aa, Artificial Sequence; identical to SEQ ID NO: 70
  Humanized VL chain

SEQ ID NO: 81 - protein, 123 aa, Artificial Sequence
  Humanized VH chain
QVQLVQSGAEVKKPGSSVKVSCKTSGDTFSSYAISWVRQAPGQGLEWMGGIIPIFGRAHYAQKFQGRVTITADESTSTAYMELSSLRSEDTAVYFCARKFHFVSGSPFGMDVWGQGTTVTVSS

SEQ ID NO: 82 - protein, 106 aa, Artificial Sequence; identical to SEQ ID NO: 5
  Humanized VL chain

SEQ ID NO: 83 - protein, 123 aa, Artificial Sequence
  Humanized VH chain
QVQLVQSGAEVKKPGSSVKVSCKTSGGTFSSYAISWVRQAPGQGLEWMGGIIPIFGKAHYAQKFQGRVTITADESTTTAYMELSSLRSEDTAVYYCARKYDYVSGSPFGMDVWGQGTTVTVSS

SEQ ID NO: 84 - protein, 106 aa, Artificial Sequence; identical to SEQ ID NO: 5
  Humanized VL chain

SEQ ID NO: 85 - protein, 273 aa, Artificial Sequence
  Tagged humanized scFv
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKAAAKPLPEVTDEYARGGPGGGGSHHHHHH

SEQ ID NO: 86 - protein, 273 aa, Artificial Sequence; identical to SEQ ID NO: 85
  Tagged humanized scFv

SEQ ID NO: 87 - protein, 271 aa, Artificial Sequence
  Tagged humanized scFv
GQVQLVQSGAEVKKPGASVKVSCKASGYTFTDYGFSWVRQAPGQGLEWMGWITAYNGNTNYAQKLQGRVTMTTDTSTSTVYMELRSLRSDDTAVYYCARDYFYGMDVWGQGTTVTVSSGGGGSGGGGSGGGGSEIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIKAAAKPLPEVTDEYARGGPGGGGSHHHHHHHH

SEQ ID NO: 88 - protein, 280 aa, Artificial Sequence
  Tagged humanized scFv
EIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIKGGGGSGGGGSGASAAGGGGSGGGGSQVQLVQSGAEVKKPGASVKVSCKASGYTFTDYGFSWVRQAPGQGLEWMGWITAYNGNTNYAQKLQGRVTMTTDTSTSTVYMELRSLRSDDTAVYYCARDYFYGMDVWGQGTTVTVSSAAAKPLPEVTDEYARGGPGGGGSHHHHHHHH

SEQ ID NO: 89 - protein, 272 aa, Artificial Sequence
  Tagged humanized scFv
GQVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKAAAKPLPEVTDEYARGGPGGGGSHHHHHHHH

SEQ ID NO: 90 - protein, 281 aa, Artificial Sequence
  Tagged humanized scFv
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKGGGGSGGGGSGASAAGGGGSGGGGSQVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSSAAAKPLPEVTDEYARGGPGGGGSHHHHHHHH

SEQ ID NO: 91 - protein, 276 aa, Artificial Sequence
  Tagged humanized scFv
GQVQLVQSGAEVKEPGSSVKVSCKASGGTFNSYAISWVRQAPGQGLEWMGGIIPLFGIAHYAQKFQGRVTITADESTNTAYMDLSSLRSEDTAVYYCARKYSYVSGSPFGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTRLEIKAAAKPLPEVTDEYARGGPGGGGSHHHHHHHH

SEQ ID NO: 92 - protein, 285 aa, Artificial Sequence
  Tagged humanized scFv
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTRLEIKGGGGSGGGGSGASAAGGGGSGGGGSQVQLVQSGAEVKEPGSSVKVSCKASGGTFNSYAISWVRQAPGQGLEWMGGIIPLFGIAHYAQKFQGRVTITADESTNTAYMDLSSLRSEDTAVYYCARKYSYVSGSPFGMDVWGQGTTVTVSSAAAKPLPEVTDEYARGGPGGGGSHHHHHHHH

SEQ ID NO: 93 - protein, 274 aa, Artificial Sequence
  Tagged humanized scFv
GQVQLVQSGAEVKKPGSSVKVSCKASGGTFSSYAINWVRQAPGQGLEWMGGIIPIFGSANYAQKFQDRVTITADESTSAAYMELSSLRSEDTAVYYCARDSSGWSRYYMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFGGGTKVEIKAAAKPLPEVTDEYARGGPGGGGSHHHHHHHH

SEQ ID NO: 94 - protein, 283 aa, Artificial Sequence
  Tagged humanized scFv
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFGGGTKVEIKGGGGSGGGGSGASAAGGGGSGGGGSQVQLVQSGAEVKKPGSSVKVSCKASGGTFSSYAINWVRQAPGQGLEWMGGIIPIFGSANYAQKFQDRVTITADESTSAAYMELSSLRSEDTAVYYCARDSSGWSRYYMDVWGQGTTVTVSSAAAKPLPEVTDEYARGGPGGGGSHHHHHHHH

SEQ ID NO: 95 - protein, 275 aa, Artificial Sequence
  Tagged humanized scFv
GQVQLVQSGAEVKKPGSSVKVSCKVSGGIFSTYAINWVRQAPGQGLEWMGGIIPIFGTANHAQKFQGRVTITADESTSTAYMELSSLRSEDTAVYYCARDQGIAAALFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKAAAKPLPEVTDEYARGGPGGGGSHHHHHHHH

SEQ ID NO: 96 - protein, 284 aa, Artificial Sequence
  Tagged humanized scFv
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGASAAGGGGSGGGGSQVQLVQSGAEVKKPGSSVKVSCKVSGGIFSTYAINWVRQAPGQGLEWMGGIIPIFGTANHAQKFQGRVTITADESTSTAYMELSSLRSEDTAVYYCARDQGIAAALFDYWGQGTLVTVSSAAAKPLPEVTDEYARGGPGGGGSHHHHHHHH

SEQ ID NO: 97 - protein, 275 aa, Artificial Sequence
  Tagged humanized scFv
GEVQLVESGGGLVQPGRSLRLSCAASGITFDDYGMHWVRQAPGKGLEWVSGISWNRGRIEYADSVKGRFTISRDNAKNSLYLQMNSLRAEDTALYYCAKGRFRYFDWFLDYWGQGTLVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIKAAAKPLPEVTDEYARGGPGGGGSHHHHHHHH

SEQ ID NO: 98 - protein, 284 aa, Artificial Sequence
  Tagged humanized scFv
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIKGGGGSGGGGSGASAAGGGGSGGGGSEVQLVESGGGLVQPGRSLRLSCAASGITFDDYGMHWVRQAPGKGLEWVSGISWNRGRIEYADSVKGRFTISRDNAKNSLYLQMNSLRAEDTALYYCAKGRFRYFDWFLDYWGQGTLVTVSSAAAKPLPEVTDEYARGGPGGGGSHHHHHHHH

SEQ ID NO: 99 - protein, 266 aa, Artificial Sequence
  Tagged humanized scFv
EVQLVESGGGLVQPGRSLRLSCAVSGFTFDDYVVHWVRQAPGKGLEWVSGISGNSGNIGYADSVKGRFTISRDNAKNSLYLQMNSLRAEDTALYYCAVPFDYWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKAAAKPLPEVTDEYARGGPGGGGSHHHHHHHH

SEQ ID NO: 100 - protein, 276 aa, Artificial Sequence
  Tagged humanized scFv
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKGGGGSGGGGSGASAAGGGGSGGGGSEVQLVESGGGLVQPGRSLRLSCAVSGFTFDDYVVHWVRQAPGKGLEWVSGISGNSGNIGYADSVKGRFTISRDNAKNSLYLQMNSLRAEDTALYYCAVPFDYWGQGTLVTVSSAAAKPLPEVTDEYARGGPGGGGSHHHHHHHH

SEQ ID NO: 101 - protein, 276 aa, Artificial Sequence
  Tagged humanized scFv
GQVQLVQSGAEVKKPGSSVKVSCKTSGDTFSSYAISWVRQAPGQGLEWMGGIIPIFGRAHYAQKFQGRVTITADESTSTAYMELSSLRSEDTAVYFCARKFHFVSGSPFGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKAAAKPLPEVTDEYARGGPGGGGSHHHHHHHH

SEQ ID NO: 102 - protein, 285 aa, Artificial Sequence
  Tagged humanized scFv
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKGGGGSGGGGSGASAAGGGGSGGGGSQVQLVQSGAEVKKPGSSVKVSCKTSGDTFSSYAISWVRQAPGQGLEWMGGIIPIFGRAHYAQKFQGRVTITADESTSTAYMELSSLRSEDTAVYFCARKFHFVSGSPFGMDVWGQGTTVTVSSAAAKPLPEVTDEYARGGPGGGGSHHHHHHHH

SEQ ID NO: 103 - protein, 276 aa, Artificial Sequence
  Tagged humanized scFv
GQVQLVQSGAEVKKPGSSVKVSCKTSGGTFSSYAISWVRQAPGQGLEWMGGIIPIFGKAHYAQKFQGRVTITADESTTTAYMELSSLRSEDTAVYYCARKYDYVSGSPFGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKAAAKPLPEVTDEYARGGPGGGGSHHHHHHHH

SEQ ID NO: 104 - protein, 285 aa, Artificial Sequence
  Tagged humanized scFv
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKGGGGSGGGGSGASAAGGGGSGGGGSQVQLVQSGAEVKKPGSSVKVSCKTSGGTFSSYAISWVRQAPGQGLEWMGGIIPIFGKAHYAQKFQGRVTITADESTTTAYMELSSLRSEDTAVYYCARKYDYVSGSPFGMDVWGQGTTVTVSSAAAKPLPEVTDEYARGGPGGGGSHHHHHHHH

SEQ ID NO: 105 - protein, 120 aa, Artificial Sequence
  Humanized VH chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 106 - protein, 110 aa, Artificial Sequence
  Humanized VL chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 107 - protein, 276 aa, Artificial Sequence
  Tagged humanized scFv
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLAAAKPLPEVTDEYARGGPGGGGSHHHHHHHH

SEQ ID NO: 108 - protein, 287 aa, Artificial Sequence
  Tagged humanized scFv
GQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGGGGSGGGGSGASAAGGGGSGGGGSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSAAAKPLPEVTDEYARGGPGGGGSHHHHHHHH